(19) United States
(12) Patent Application Publication
(10) Pub. No.: US 2022/0293209 A1
(43) Pub. Date: Sep. 15, 2022

(54) GENOMIC AND EPIGENOMIC COMPARATIVE, INTEGRATIVE PATHWAY DISCOVERY

(71) Applicant: Rutgers, The State University of New Jersey, New Brunswick, NJ (US)

(72) Inventors: Antonina Mitrofanova, New York, NY (US); Nusrat J. Epsi, Newark, NJ (US)

(73) Assignee: Rutgers, The State University of New Jersey, New Brunswick, NJ (US)

(21) Appl. No.: 17/624,181

(22) PCT Filed: Jun. 30, 2020
(86) PCT No.: PCT/US2020/040379

§ 371 Date: Dec. 30, 2021

Related U.S. Application Data
(60) Provisional application No. 62/869,503, filed on Jul. 1, 2019.

(51) Int. Cl. G16B 5/00; G16B 20/00

(52) U.S. Cl. CPC G16B 5/00; G16B 20/00

(57) ABSTRACT

Disclosed herein are systems and methods for identifying biomarkers. Biomarker identification can be achieved while increasing efficiency and decreasing data and computation complexity but maintaining accuracy. Such biomarker identification can be achieved by applying pathway enrichment analysis associated with differential gene expression and epigenomic regulation, such as DNA methylation.

### CROSS-REFERENCE TO RELATED APPLICATION

This application claims priority to U.S. Provisional Application No. 62/869,503, filed Jul. 1, 2019, herein incorporated by reference in its entirety.

### FIELD

The field relates to biomarker-identifying technologies implemented via gene enrichment analysis, such as by query and reference inputs.

### BACKGROUND

Biomarker-identifying systems and methods are a fundamental problem faced by physicians and researchers. Although a number of techniques have been developed to increase efficiency of biomarker identification, there remains room for improvement.

### SUMMARY

The Summary is provided to introduce a selection of concepts in a simplified form that are further described below in the Detailed Description. The Summary is not intended to identify key features or essential features of the claimed subject matter, nor is it intended to be used to limit the scope of the claimed subject matter.

In one embodiment, a computer-implemented method of identifying treatment-response biomarkers comprises receiving genomic and epigenomic datasets for at least two subjects, wherein the at least two subjects have different treatment-response phenotypes; identifying differential genomic and epigenomic datapoints in the datasets, wherein the identifying generates at least one differential genomic signature for the treatment-response phenotypes and at least one differential epigenomic signature for the treatment-response phenotypes; determining biological pathways enriched in the at least one differential genomic signature and the at least one differential epigenomic signature, wherein the determining generates at least one genomic pathway signature and at least one epigenomic pathway signature; and selecting the biological pathways enriched between the at least one genomic pathway signature and the at least one epigenomic pathway signature, wherein the selecting generates a comprehensive pathway signature for the treatment-response phenotypes.

In another embodiment, a treatment-response biomarker identification system comprises one or more processors; and memory coupled to the one or more processors, wherein the memory comprises computer-executable instructions causing the one or more processors to perform a process comprising receiving genomic and epigenomic datasets for at least two subjects, wherein the at least two subjects have different treatment-response phenotypes; identifying differential genomic and epigenomic datapoints in the datasets, wherein the identifying generates at least one differential genomic signature for the treatment-response phenotypes and at least one differential epigenomic signature for the treatment-response phenotypes; determining biological pathways enriched in the at least one differential genomic signature and the at least one differential epigenomic signature, wherein the determining generates at least one genomic pathway signature and at least one epigenomic pathway signature; and selecting pathways enriched between the at least one genomic pathway signature and the at least one epigenomic pathway signature, wherein the selecting generates a comprehensive pathway signature for the treatment-response phenotypes.

In a further embodiment, one or more computer-readable media have encoded thereon computer-executable instructions that, when executed, cause a computing system to perform a treatment-response biomarker identification method comprising receiving genomic and epigenomic datasets for at least 2 subjects, wherein the at least 2 subjects have different treatment-response phenotypes; identifying differential genomic and epigenomic datapoints in the datasets, wherein the identifying generates at least one differential genomic signature for the treatment-response phenotypes and at least one differential epigenomic signature for the treatment-response phenotypes; determining biological pathways enriched in the at least one differential genomic signature and the at least one differential epigenomic signature, wherein the determining generates at least one genomic pathway signature and at least one epigenomic pathway signature; and selecting pathways enriched between the at least one genomic pathway signature and the at least one epigenomic pathway signature, wherein the selecting generates a comprehensive pathway signature for the treatment-response phenotypes.

As described herein, a variety of other features and advantages can be incorporated into the technologies as desired.

### BRIEF DESCRIPTION OF THE DRAWINGS

FIG. 1 is a block diagram of an example system identifying treatment-response biomarkers.

FIG. 2 is a flowchart of an example method identifying treatment-response biomarkers.

FIG. 3 is a block diagram of an example system performing pathway enrichment and combination.

FIG. 4 is a block diagram of an example system generating a pathway signature for a pathway collection.

FIG. 5 is a block diagram of an example system calculating transcriptomic and epigenomic pathway signatures.

FIG. 6 is a flowchart of an example method calculating transcriptomic and epigenomic pathway signatures.

FIG. 7 is a block diagram of an example system calculating a composite pathway signature.

FIG. 8 is a block diagram of an integrative pathway analysis.

FIG. 9 shows a schematic representation of an example pathway altered at both genomic and epigenomic levels.

FIGS. 10A-D show an example integrative systematic epigenomic analysis that identifies candidate molecular pathways for a chemotherapy response.

FIGS. 11A-B show example epigenomic alterations in candidate molecular pathways of carboplatin-paclitaxel response.

FIGS. 12A-D show that example candidate molecular pathways stratify patients based on response to carboplatin-taxane in an independent cohort.

FIGS. 13A-D show an example comparative performance analysis that confirms the significant predictive ability of the technologies described herein.

FIGS. 14A-C show that an example implementation (pathCHEMO) accurately identifies pathways of treatment resistance across chemo-regimens and cancer types.

FIG. 15 shows an example schematic flow representation of an example implementation (pathCHEMO).

FIG. 16A-C shows that comparative testing of treatment response signatures demonstrates their robustness.

FIG. 17 shows example epigenomic alterations in selected candidate molecular pathways of carboplatin-paclitaxel resistance.

FIGS. 18A-B shows an example region-based analysis of differentially methylated sites in 7 candidate pathways.

FIGS. 19A-G show example candidate molecular pathways for predicting a response to carboplatin-taxane but that are not predictive of lung cancer aggressiveness.

FIGS. 20A-C show stratified Kaplan-Meier survival analysis demonstrates independence of the candidate pathways from the common covariants.

FIG. 21 shows example networks of proteins and pathways affected by lung adenocarcinoma cancer and carboplatin-taxane chemotherapy. Larger circles indicate that the protein is more affected by the cancer and chemotherapy.

FIGS. 22A-C show example identification of pathways of treatment resistance across chemo-regimens and cancer types.

FIG. 23 is a box and whisker plot depicting p-value cutoff and Normalized Enrichment Score. 10321 FIG. 24 is a graph of gene set enrichment analysis comparing expression pathway signature and methylation pathway signature.

FIG. 25 is a graph of survival analysis estimates in response to carboplatin-taxane.

FIG. 26 is a graph of two random models that indicate non-random predictive ability of the technologies described herein.

FIG. 27 is a graph of treatment related survival analysis in cisplatin-vinorelbine treated lung adeno-carcinoma (LUAD) patients.

FIG. 28 is a graph of treatment related survival analysis in cisplatin-vinorelbine treated lung squamous cell carcinoma (LUSC) patients.

FIG. 29 is a graph of treatment related survival analysis in FOLFOX (folinic acid, uorouracil, and oxaliplatin) treated colorectal adenocarcinoma (COAD) patients.

FIG. 30 is a block diagram of an example computing system in which described embodiments can be implemented.

FIG. 31 is a block diagram of an example cloud computing environment that can be used in conjunction with the technologies described herein.

### DETAILED DESCRIPTION

### Example 1—Overview

A wide variety of genomic and epigenomic pathway enrichment techniques can be used that implement identifying biomarkers of phenotypes (such as treatment-response phenotypes). The resulting biomarkers can be used for treating subjects. Implementations of identifying biomarkers of phenotypes (such as treatment-response phenotypes) using such genomic and epigenomic pathway enrichment techniques can be used to stratify patients based on phenotype, such as a negative or positive treatment response, which can indicate treatment failure or resistance. Accordingly, stratifying patients based on phenotype (such as treatment-response phenotypes) will improve disease course and provide better informed clinical decision making.

### Example 2—Example Biomarkers

In any of the examples herein, the described biomarkers can take the form of a pathway (e.g., affected on both transcriptomic and epigenomic levels as described herein). In practice, a pathway can comprise a set of a plurality of gene identifiers that identify real-world genes as described herein. Such genes are grouped together in the pathway by their involvement in the same biological pathway, or by proximal location on a chromosome. As described herein, databases of such sets of genes can be used as an input pathway collection. The technologies herein can comprise identifying (e.g., discovering) candidate pathways out of such databases, where the identifying comprises selecting (e.g., filtering) a set of pathways based on enrichment scores as described herein.

### Example 3—Examples System Implementing Identifying Biomarkers of Phenotypes

Example systems for implementing identifying biomarkers of phenotypes (such as treatment-response phenotypes) via genomic and/or epigenomic pathway enrichment are disclosed herein. Example systems can include a processor coupled to memory, such as memory with computer-executable instructions for identifying treatment-response biomarkers.

Example systems can include training and use of genomic and/or epigenomic data via genomic and/or epigenomic pathway enrichment to generate biomarkers, such as a pathway signature, for identification of phenotypes (such as treatment-response phenotypes). In practice, genomic and/or epigenomic pathway enrichment can be trained and used independently or in tandem. For example, a system can be trained and then deployed to be used independent of any training activity, or the system can continue to be used after deployment. In practice, the system can receive genomic and/or epigenomic data, which can be used to generate a pathway signature for one or more phenotypes (such as treatment-response phenotypes). The system can then receive additional genomic and/or epigenomic data, for which a pathway signature can be used via genomic and/or epigenomic pathway enrichment to determine one or more phenotypes (such as treatment-response phenotypes).

In practice, a system receives genomic and/or epigenomic data for at least one subject or group of subjects. The subject or group can have a known or an unknown phenotype (such as treatment-response phenotypes), such as for system training or use.

In examples, a system can use genomic and/or epigenomic data to identify differential genomic and/or epigenomic datapoints. Differential genomic and/or epigenomic signatures can also be generated. Various types of signatures are possible with various indicia of differentiation.

In examples, a system can use pathway enrichment to determine enriched biological pathways based on differential genomic and/or epigenomic signatures. Pathway signatures can also be generated, such as genomic and/or epigenomic pathway signatures.

In examples, a system can further use pathway enrichment to select biological pathways enriched among enriched genomic and epigenomic pathways, such as using genomic and epigenomic pathway signatures. Comprehensive pathway signatures, such as pathway signatures that include enriched genomic and epigenomic pathways, can also be generated.

In practice, the systems disclosed herein can vary in complexity with additional functionality, more complex components, and the like. The described systems can also be networked via wired or wireless network connections to a global computer network (e.g., the Internet). Alternatively, systems can be connected through an intranet connection (e.g., in a corporate environment, government environment, educational environment, research environment, or the like).

The systems disclosed herein can be implemented in conjunction with any of the hardware components described herein, such as computing systems described below (e.g., processing units, memory, and the like). In any of the examples herein, the inputs, outputs, signatures (such as differential genomic and epigenomic signatures, genomic and epigenomic pathway signatures, or comprehensive pathway signatures), trained identifiers (such as pathway enrichment identifiers), information about signatures (such as genomic and epigenomic data or information about differential genomic and epigenomic signatures, genomic and epigenomic pathway signatures, and comprehensive pathway signatures), and the like can be stored in one or more computer-readable storage media or computer-readable storage devices. The technologies described herein can be generic to the specifics of operating systems or hardware and can be applied in any variety of environments to take advantage of the described features.

### Example 4—Example Method Implementing Identifying Treatment-Response Biomarkers

Example methods implementing identifying biomarkers of phenotypes (such as treatment-response phenotypes) are disclosed herein.

Example methods include both training and use of genomic and epigenomic data via genomic and epigenomic pathway enrichment to generate biomarkers, such as pathway signatures, for phenotype identification (such as identification of treatment response phenotypes). However, in practice, either phase of the technology can be used independently (e.g., a system can be trained and then deployed to be used independently of any training activity) or in tandem (e.g., training continues after deployment).

In examples, a genomic and/or epigenomic data are received. Genomic and epigenomic data can take the form described herein.

Further, genomic and/or epigenomic data can be received with or without additional processing. For examples, the method can include normalizing, transforming, or reducing redundancy in the data. Other processing steps are possible.

In examples, the methods can include generating differential genomic and/or epigenomic signatures using genomic and/or epigenomic data (such as by identifying, for example using a differential identifier). In practice, genomic and/or epigenomic data input into a differential identifier, and differential genomic and/or epigenomic signatures are output.

In examples, the methods can include generating pathway signatures using differential genomic and/or epigenomic data, such as by determining (for example, using a pathway enrichment identifier). In practice, differential genomic and/or epigenomic signatures can be input into a pathway enrichment identifier, and genomic and/or epigenomic pathway signatures can be output.

In examples, the methods can include generating a comprehensive pathway signature, such as by determining (for example, using a comprehensive pathway enrichment identifier). In practice, genomic and/or epigenomic pathway signatures (such as composite genomic and/or epigenomic pathway signatures) can be input into a comprehensive pathway enrichment identifier, and a comprehensive pathway signature can be output.

### Example 5—Example Genomic Data

In any of the examples herein, genomic data can take a variety of forms. For example, genomic data can include level of expression associated with a gene, such as a list of one or more genes or set of genes, in which each gene is associated with a level of expression. In practice, digital genomic data or a digital representation of genomic data can be used as input to the technologies. In practice, genomic data can take the form of a digital or electronic item such as a file, binary object, digital resource, or the like.

Example genomic data can include gene or gene expression data, such as a direct or an indirect measure of genes or gene expression. For example, transcriptomic or proteomic data can be used as a measure of gene expression. In specific, non-limiting examples, genomic data can include nucleic acid-based data, such as mRNA or miRNA data, or protein expression data.

Data obtained using various techniques can be used in the methods herein. For example, nucleic acid-based data can be obtained, such as hybridization (such as array-, chip-, or barcoding-based hybridization) or polymerase chain reaction (PCR, such as reverse transcription (RT)-PCR, including quantitative RT-PCR, or qRT-PCR) data, or protein expression data can be obtained, such as mass spectrometry, electrophoresis, array-based, or chromatography data. In specific, non-limiting examples, data can comprise nucleic acid-based data, such as mRNA or miRNA data, for example, array- or chip-based data.

Genomic data can further include gene or gene expression data from a variety of sources, such as private or publicly accessible databases. For example, databases can include general or specialized databases, such as databases specific for species, taxa, or subject, for example, cancer subjects (such as the Cancer Genome Atlas or the Genomics Data Commons database, portal.gdc.cancer.gov).

Further, in any of the examples herein, genomic data can be used with or without additional processing. For example, the methods can include normalization or variance-stabilizing transformation. Other processing is possible, such as centering, standardization, log transformation, rank transformation, and the like.

In any of the examples herein, genomic data or its representation can be stored in a database (such as a genomic data database). The database can include genomic data with or without additional processing. In particular examples, genomic data are stored as a raw or processed RNA-seq data (such as RNA-seq counts, for example, normalized or transformed RNA-seq counts). Precompiled genomic data databases may also be used. For example, an application that already has access to a database of pre-computed genomic data can take advantage of the technologies without having to compile such a database. Such a database can be available locally, at a server, in the cloud, or the like. In practice, a different storage mechanism than a database can be used (such as a sequence table, index, or the like).

### Example 6—Example Epigenomic Data

In any of the examples herein, epigenomic data can take a variety of forms. For example, epigenomic data can include level of epigenomic activity (such as methylation) associated with a gene, such as a list of one or more genes or set of genes, in which each gene is associated with a level of epigenomic activity (such as methylation). In practice, digital epigenomic data or a digital representation of epigenomic data can be used as input to the technologies. In practice, epigenomic data can take the form of a digital or electronic item such as a file, binary object, digital resource, or the like.

Example epigenomic data can take a variety of forms. In examples, epigenomic data include DNA methylation data, such as a direct or an indirect measure of DNA methylation or genome-wide or site-specific DNA methylation data. For example, DNA methylation data can include a measure of a label or probe of DNA methylation (such as the intensity or a count of a label or probe of DNA methylation).

Data obtained using various techniques can be used in the methods herein. For example, data obtained from bisulfite sequencing or conversion, pyrosequencing, HPLC-UV, LC-MS/MS, ELISA-based methods, and array- or bead-based hybridization techniques can be used. In specific, non-limiting examples, data can include DNA methylation array- or chip-based data.

Epigenomic data can further include gene or gene expression data from a variety of sources, such as private or publicly accessible databases. For example, databases can include general or specialized databases, such as databases specific for species, taxa, or subject, for example, cancer subjects (such as the Cancer Genome Atlas or the Genomics Data Commons database, portal.gdc.cancer.gov).

Further, in any of the examples herein, epigenomic data can be used with or without additional processing. For example, the methods can include normalization, transformation (such as transformation of DNA methylation data to β values or M values), or redundancy reduction (such as a by selecting a single site one methylation site per gene, such as one CpG site per gene, for example, based on statistical factor, such as a highest coefficient of variation). Other processing is possible, such as standardization, logit transformation, bias correction, and the like.

In any of the examples herein, epigenomic data or its representation can be stored in a database (such as an epigenomic data database). The database can include epigenomic data with or without a preprocessing step. In particular examples, epigenomic data are stored as a raw or processed DNA methylation intensity or count data (such as count or intensity of a label or probe for DNA methylation, for example, as β value or M values). Precompiled epigenomic data databases may also be used. For example, an application that already has access to a database of pre-computed epigenomic data can take advantage of the technologies without having to compile such a database. Such a database can be available locally, at a server, in the cloud, or the like. In practice, a different storage mechanism than a database can be used (such as a methylation table, index, or the like).

### Example 7—Example Subjects

In any of the examples herein, genomic or epigenomic data can include data for a variety of subjects or groups of subjects. In practice, subjects can be single subjects or a part of a group (such as a group with a common feature or characteristic, or a cohort).

In examples, data for subjects or groups can be used for training. For example, subjects or groups can include known features or phenotypes, such as for training and validation thereof (for example, training or validation subjects, groups, or cohorts). In specific, non-limiting examples, subjects or groups have a disease, such as cancer or a specific type of cancer (or a malignant tumor characterized by abnormal or uncontrolled cell growth, such as reproductive, gastrointestinal, pancreatic, blood, kidney, bladder, skin, head and neck, nervous system, bone and cartilage, lymphatic, lung, or colorectal cancer, for example, lung adenocarcinoma, lung squamous cell carcinoma, or colon adenocarcinoma), or a treatment response phenotype (such as a chemotherapy treatment response phenotype, for example, a response to a chemical or biological agent with therapeutic usefulness in the treatment of diseases characterized by abnormal cell growth, for example, carboplatin, paclitaxel, cisplatin, vinorelbine, folinic acid, fluorouracil, oxaliplatin, or a combination thereof).

In examples, data for subjects or groups can be used to identify subjects with a feature or phenotype. In practice, subjects or groups can include unknown features or phenotypes, which can then be identified using a trained system (for example, query subjects, groups or cohorts). For example, subjects or groups can have a disease, such as cancer or a specific type of cancer (or a malignant tumor characterized by abnormal or uncontrolled cell growth, such as reproductive, gastrointestinal, pancreatic, blood, kidney, bladder, skin, head and neck, nervous system, bone and cartilage, lymphatic, lung, or colorectal cancer, for example, lung adenocarcinoma, lung squamous cell carcinoma, or colon adenocarcinoma), and a trained system can be used to identify subjects or groups with a treatment response phenotype (such as a chemotherapy treatment response phenotype, for example, a response to a chemical or biological agent with therapeutic usefulness in the treatment of diseases characterized by abnormal cell growth, for example, carboplatin, paclitaxel, cisplatin, vinorelbine, folinic acid, fluorouracil, oxaliplatin, or a combination thereof).

### Example 8—Example Treatment-Response Phenotypes

In any of the examples herein, treatment-response phenotypes can include a variety of phenotypes. In practice, treatment-response phenotypes can depend on a variety of factors, including gene expression and epigenomic regulation (such as DNA methylation). Therefore, in examples, gene expression and/or epigenomic data can be used in the examples herein to identify treatment response phenotypes.

Treatment-response phenotypes for a variety of diseases can be identified in the examples herein. For example, diseases can include cancers, such as specific types of cancers (or a malignant tumor characterized by abnormal or uncontrolled cell growth, such as reproductive, gastrointestinal, pancreatic, blood, kidney, bladder, skin, head and neck, nervous system, bone and cartilage, lymphatic, lung, or colorectal cancer, for example, lung adenocarcinoma, lung squamous cell carcinoma, or colon adenocarcinoma).

Treatment-response phenotypes for a variety of treatments can be identified in the examples herein. For example, treatments can include chemotherapy, such as (such as a chemotherapy treatment response phenotype, for example, a response to a chemical or biological agent with therapeutic usefulness in the treatment of diseases characterized by abnormal cell growth, for example, carboplatin, paclitaxel, cisplatin, vinorelbine, folinic acid, fluorouracil, oxaliplatin, or a combination thereof).

In practice, treatment responses can include a positive treatment response or failure. For example, a positive treatment response can include amelioration of a sign or symptom of a disease or pathological condition after it has begun to develop. In specific, non-limiting examples, a positive response to treatment includes a positive response to chemotherapy, such as in a subject that does not develop resistance to chemotherapy treatment. In examples, failure can include failure to ameliorate a sign or symptom of a disease or pathological condition after it has begun to develop. In specific, non-limiting examples, failure can include failure of chemotherapy treatment, such as in a subject who develops resistance to chemotherapy treatment.

### Example 9—Example Differential Genomic and Epigenomic Signature

In any of the examples herein, a variety of differential genomic and epigenomic signatures can be used. In practice, one or more than one differential genomic and epigenomic signature can be used. In examples, more than one differential genomic and epigenomic signature can be used, such as during training. In examples, a single sample genomic and epigenomic signature can be used, such as during use or validation.

In practice, differential genomic signatures and differential epigenomic signatures can include various genes or sets of genes. For example, a targeted set of genes or a genome-wide set of genes can be included.

In examples, differential genomic signatures can include differential expression of genes or sets of genes. For example, genes in which an amount of one or more of its expression products (for example, transcripts, such as mRNA, and/or protein) is higher or lower in one sample (such as a test sample) as compared to another sample (such as a control sample or a reference standard, for example, a healthy subject or subjects or a subject or subjects with a disease and/or treatment response phenotype, such as a subject or subjects who respond positively to chemotherapy, or a subject or subjects who do not develop resistance to chemotherapy, such as a subject who does not respond positively to chemotherapy, such as a subject or subjects who develop resistance to chemotherapy, or a historical control or standard reference value or range of values). In practice, differential expression can include an increase or a decrease in expression of a gene or genes. Differential expression can include a quantitative increase or a decrease in expression, for example, a statistically significant increase or decrease.

In examples, differential genomic signatures can include differential methylation of genes or sets of genes. For example, differential methylation can include nucleotides in a gene (such as the gene body) or sequences associated with gene transcription (such as promoters, for example, in CpG islands of promoters) in which methylation is higher or lower in one sample (such as a test sample) as compared to another sample (such as a control) as compared to another sample (such as a control sample or a reference standard, for example, a healthy subject or subjects or a subject or subjects with a disease and/or treatment response phenotype, such as a subject or subjects who respond positively to chemotherapy, or a subject or subjects who do not develop resistance to chemotherapy, such as a subject who does not respond positively to chemotherapy, such as a subject or subjects who develop resistance to chemotherapy, or a historical control or standard reference value or range of values). In practice, differential methylation can include an increase or a decrease in methylation of DNA for a gene or genes. Differential methylation can include a quantitative increase or a decrease in methylation, for example a statistically significant increase or decrease.

In examples, various methods can be used to identify differential genes for differential genomic signatures and differential epigenomic signatures. For example, genomic or epigenomic data (such as described herein) for a genes or a set of genes can be compared.

In examples, the differential genomic signatures and differential epigenomic signatures can take a variety of forms. For example, a ranked list (such as based on level of differentiation), a list of genes with significance assigned, or a list of genes that meet an applied cut-off threshold (such as based on level of differentiation). Other forms are possible. For example, where gene differentiation is quantified (for example, producing positive values for overexpression or over-methylation and producing negative values for underexpression or under-methylation), differential genomic signatures and differential epigenomic signatures can include absolute valued differential genomic signatures and differential epigenomic signatures or signed differential genomic signatures and differential epigenomic signatures.

### Example 10—Example Genomic and Epigenomic Pathway Signatures

In any of the examples herein, a variety of genomic and epigenomic pathway signatures can be used.

In practice, genomic and epigenomic pathway signatures can take a variety of forms. For example, genomic and epigenomic pathway signatures can include a list of pathways enriched in differential genomic signatures and differential epigenomic signatures. In practice, the list of pathways can include a variety of possible pathways. In examples, possible pathways can include the pathways listed in one or more general or specific pathway databases (for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like, such as described in Garcia-Campos et al., Front. Physiol, 6(383), 2015, incorporated herein by reference in its entirety), such as during training. In examples, possible pathways can include pathways listed in a comprehensive pathway signature (such as comprehensive pathway signatures disclosed herein), such as during use or validation, for example, in single sample genomic and epigenomic pathway signatures or single sample composite genomic and epigenomic pathway signatures.

In examples, enriched pathways can be quantified based on the level of enrichment in differential genomic signatures and differential epigenomic signatures. Other forms are possible.

In examples, genomic and epigenomic pathway signatures can be generated based on absolute valued differential genomic signatures and differential epigenomic signatures or signed differential genomic signatures and differential epigenomic signatures. Thus, genomic and epigenomic pathway signatures can also include absolute valued genomic pathway signatures and epigenomic pathway signatures or signed genomic pathway signatures and epigenomic pathway signatures. Single sample genomic and epigenomic pathway signatures can also be signed or absolute valued.

In examples, genomic and epigenomic pathway signatures can include multiple genomic pathway signatures or multiple epigenomic pathway signatures, such as in composite genomic and epigenomic pathway signatures. For example, genomic pathway signatures can include absolute valued genomic pathway signatures and signed genomic pathway signatures in composite genomic pathway signatures. Further, epigenomic pathway signatures can include absolute valued epigenomic pathway signatures and signed epigenomic pathway signatures in composite epigenomic pathway signatures. Composite genomic and epigenomic pathway signatures can include all or some information from multiple genomic or epigenomic pathway signatures. For example, composite genomic and epigenomic pathway signatures can include a list of enriched pathways that have been quantified, wherein a highest level of enrichment for each pathway among the combined pathway signatures can be associated with each pathway in the composite genomic and epigenomic pathway signatures.

### Example 11—Example Comprehensive Pathway Signatures

In any of the examples herein, a variety of comprehensive pathway signatures can be used.

In examples, comprehensive pathway signatures can include a list of pathways enriched in at least two biological phenomena. In practice, the list of pathways can include a variety of possible pathways. In examples, possible pathways can include the pathways listed in one or more genomic or epigenomic pathway signatures, such as composite genomic and epigenomic pathway signatures (for example, single sample composite genomic and epigenomic pathway signatures, such as in use or validation).

Comprehensive pathway signatures can take a variety of forms. For example, comprehensive pathway signatures can include pathways with certain features or characteristics, such as pathways enriched at least two biological phenomena or pathways with statistical significance (for example, with a p value of less than 0.05, less than 0.01, less than 0.005, or less than 0.001). Comprehensive pathways can also exclude pathways with certain features or characteristics, such as redundant pathways (such as pathways with more than one convention, for example, pathways that are included in both narrowly and broadly defined conventions).

### Example 12—Example System Identifying Treatment-Response Biomarkers

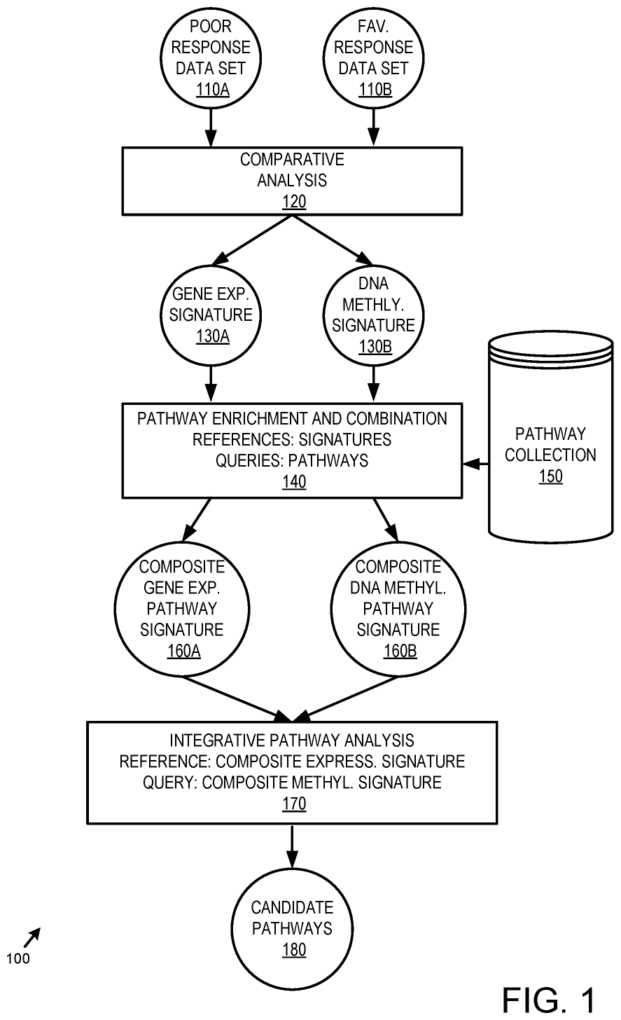

FIG. 1 is a block diagram a basic system 100 that can be used to implement identification of treatment-response biomarkers as described herein. The system 100 can be implemented in a computing system as described herein. In the example, a comparative analysis 120 receives both a poor response data set 110A and a favorable response data set 110B and outputs both a gene expression signature 130A and DNA methylation signature 130B, which then serve as input along with the pathway collection 150 to pathway enrichment and combination 140 (e.g., which treats the inputs as references and queries). Such signatures can comprise ranked values for multiple genes. Patients with poor and favorable response for each gene can be compared so that scores, ranks, or both of the genes can reflect a given gene's differential expression between the patient groups.

The outputs of the pathway enrichment and combination 140 comprise a composite gene expression pathway signature 160A and a composite DNA methylation pathway signature 160B, which are then used as input for integrative pathway analysis 170 (e.g., which treats the inputs as references and queries), which outputs the candidate pathways 180.

Although the example shows poor and favorable response data sets, in practice, patients across a spectrum response (e.g., poor, favorable, intermediate treatment response) can be included. Pathway activities can be estimated in individual patients (e.g., per-patient pathway activity analysis) and then correlated or associated to therapeutic response.

In practice, the systems shown herein, such as system 100, can vary in complexity, with additional functionality, more complex components, and the like. For example, there can be additional functionality within integrative pathway analysis 170. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 100 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

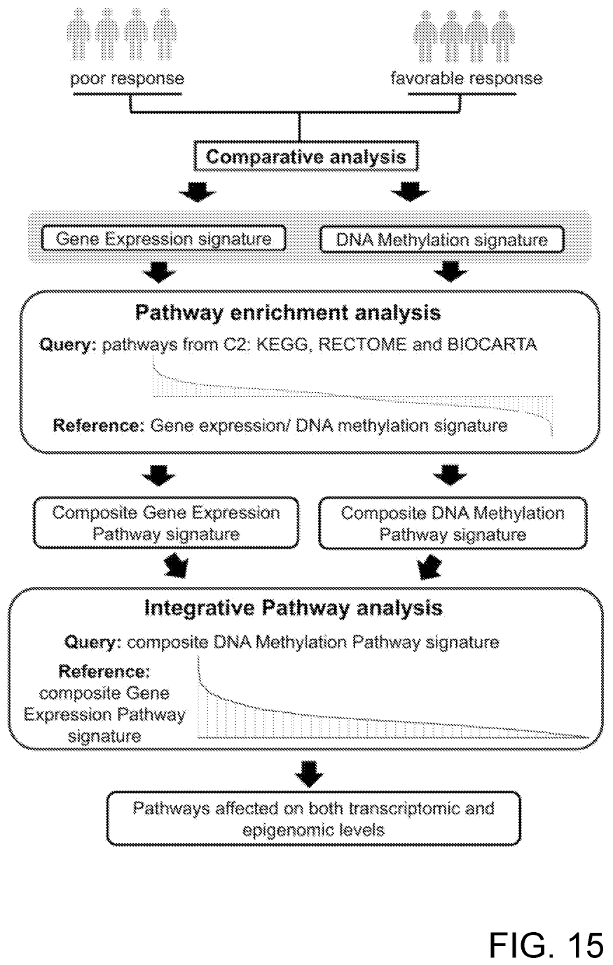

An alternative arrangement is shown in FIG. 15, which is referenced below.

### Example 13—Example Method Identifying Treatment-Response Biomarkers

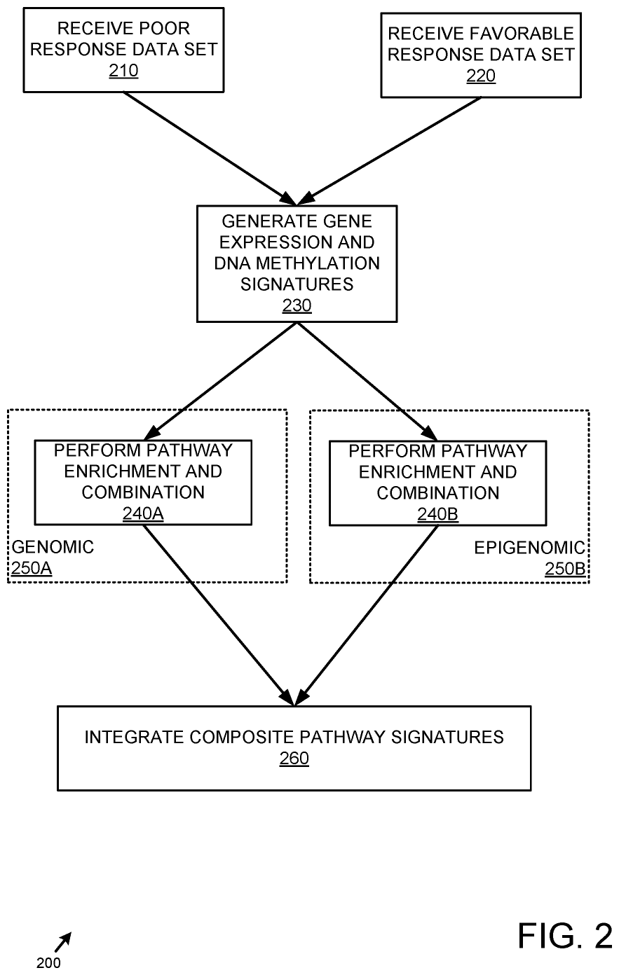

FIG. 2 is a flowchart of an example method 200 identifying treatment-response biomarkers and can be implemented, for example, in the system of that shown in FIG. 1.

In the example, at 210, a poor response data set is received. At 220, a favorable response data set is received. In practice, one can also include other response levels (e.g., intermediate response).

At 230, gene expression and DNA methylation signatures are generated, such as via scaling (e.g., via a Z-score). When computing a signature, one can z-score the data, using the z-score as a signature and perform pathway enrichment analysis on a given patient (for a plurality of patients), then associate pathway activities with therapeutic response. Scaling can be performed to facilitate estimating pathway activity changes per patient (e.g., data standardization). Such a technique can be used for validation, and also for discovery in a per-patient analysis.

At 240A, pathway enrichment and combination is performed at the genomic level 250A as described herein. At 250B, pathway enrichment and combination is performed at the epigenomic level 250B as described herein.

Subsequently, composite pathway signatures are integrated and analyzed at 260. During integrative pathway analysis, pathways affected on both the transcriptomic and epigenomic levels can be selected (e.g., discovered) as candidate pathways. As described herein the method 200 has been successful in identifying useful candidate pathways.

The method 200 can incorporate any of the methods or acts by systems described herein to achieve the described technologies.

The method 200 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### Example 14—Example Pathway Enrichment

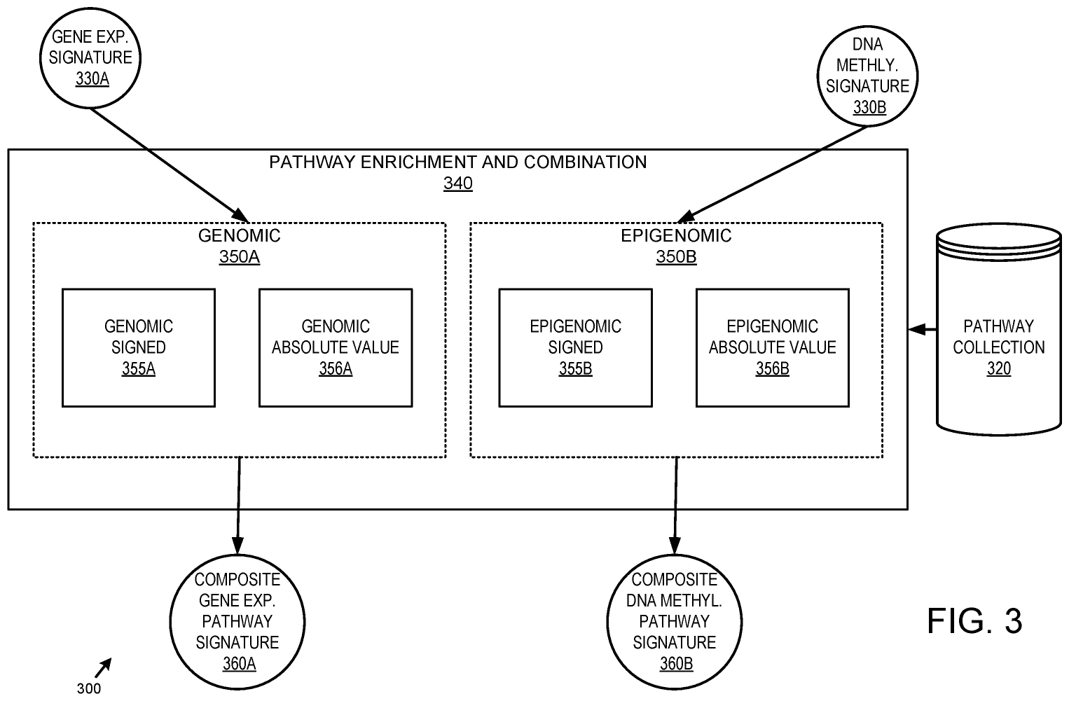

FIG. 3 is a block diagram of an example system 300 performing pathway enrichment and combination that can be used in any of the examples herein.

Pathway enrichment and combination 340 receives gene expression signature 330A, DNA methylation signature 330B, and pathway collection 320 as input and provides composite gene expression pathway signature 360A and composite DNA methylation pathway signature 360B as output.

As shown, enrichment and combination can be performed both on the genomic 350A and Epigenomic 350B sides via both signed 355A, 355B and absolute value 356A, 356B analyses as described herein.

### Example 15—Example Pathway Signature Generation

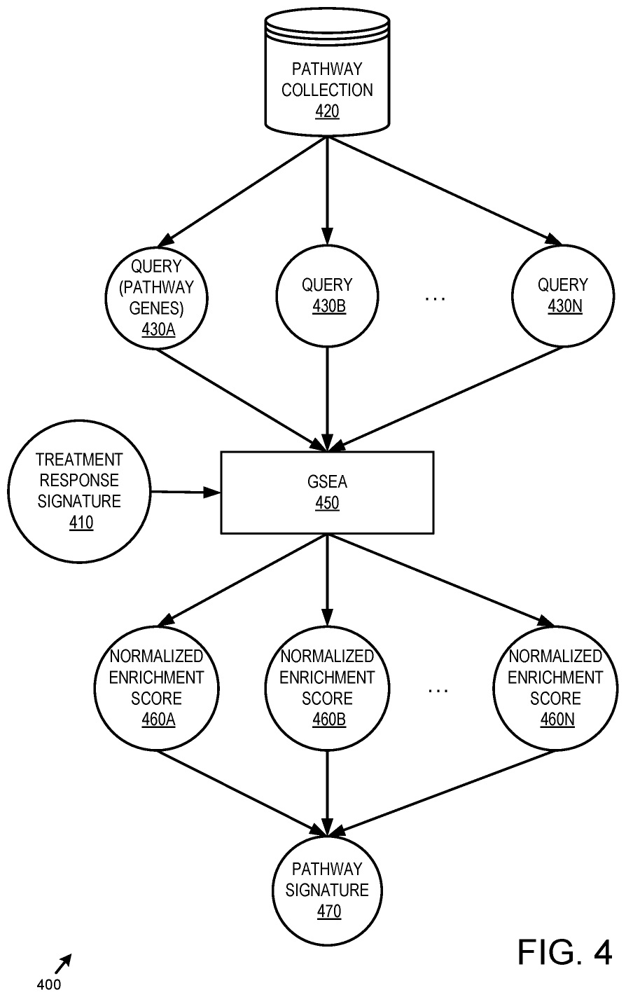

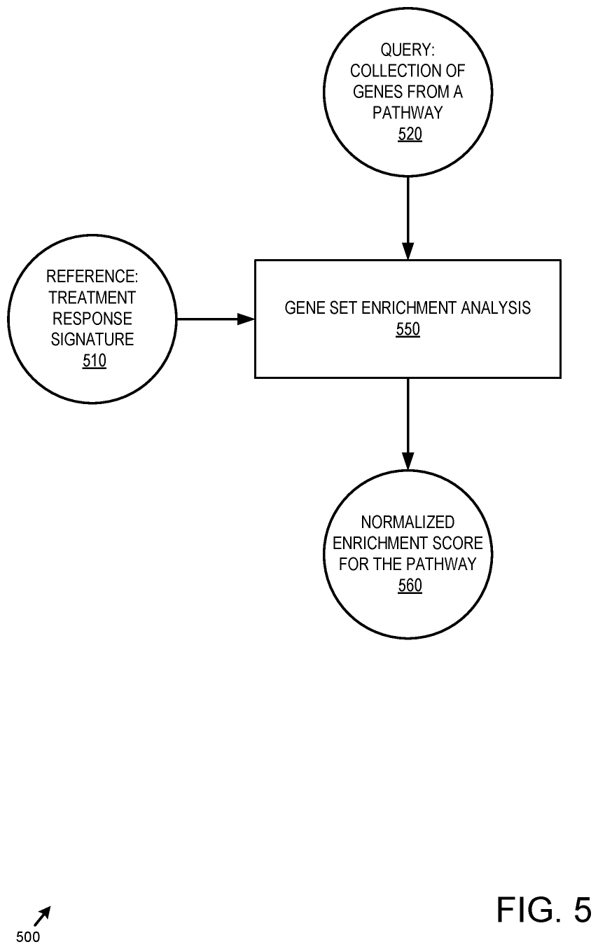

FIG. 4 is a block diagram of an example system 400 generating a pathway signature 470 that can be used in any of the examples herein.

In the example, a pathway collection 420 (e.g., of a plurality of pathways) is used to generate a plurality of queries 430A-N (using pathway genes), where a given query (e.g., 430A) uses the genes from a respective pathway out of the collection 420. Gene set enrichment analysis (GSEA) 450 can be performed on the results along with a treatment response signature 410 as input (a reference), resulting in normalized enrichment scores 460A-N (e.g., a score for a corresponding pathway, such as a score 460A for the query 430A for a respective pathway out of the collection 420), which are combined into the pathway signature 470.

A treatment response gene expression/methylation signature can take the form of a list of genes/methylation sites ranked by their differential expression/methylation between any two phenotypes of interest. Ranking/scoring of genes/sites can be accomplished using any appropriate statistics (e.g., t-test, fold change, or the like) that compares any two phenotypes of interest (e.g., responder versus non-responder patients). A treatment response signature can be defined for a given cancer type (e.g., lung adenocarcinoma) for respective treatments (e.g., carboplatin and paclitaxel treatment) for respective data types (e.g., gene expression or DNA methylation).

A treatment response pathway gene expression/methylation signature can take the form of a list of pathways ranked by their activity levels between any two phenotypes of interest. Ranking/scoring of a pathway can be done using signed and absolute-valued GSEA, where Normalized Enrichment Score (NEC) and p-value reflects pathway activity changes between two phone types of interested (e.g., responder versus non-responder patients). A treatment response pathway signature can be defined for a given cancer type (e.g., lung adenocarcinoma) for respective treatments (e.g., carboplatin and paclitaxel treatment) for respective data types (e.g., gene expression or DNA methylation). A composite pathway signature can integrate treatment response pathway signatures from different data types (e.g., gene expression and DNA methylation).

In practice, the analysis can be run four times: two times for gene expression signature (signed and absolute valued analysis) and two times for DNA methylation signature (signed and absolute valued analysis). Four pathway signatures 470 can thus result.

### Example 16—Example Pathway Signature Calculation

FIG. 5 is a block diagram of an example system 500 calculating transcriptomic and epigenomic pathway signatures that can be used in any of the examples herein.

In the example, GSEA 550 is performed using a collection of genes 520 from a pathway as a query and a treatment response signature 510 as a reference, outputting a normalized enrichment score 560 for the pathway. In practice the analysis can be performed with one iteration for one pathway (e.g., n iterations for n pathways).

The system 500 is useful for pathway enrichment analysis and can be used as a basis for FIG. 4 (e.g., an atomic operation for FIG. 4 that is performed a plurality of times).

As shown in the example, the treatment response signature is used as a reference and a collection of genes from a pathway is used as a query. In practice, the operations estimate where genes from a pathway fall on the signature. The operations walk the signature (because the signature is sorted by its ranked values) and note when a pathway gene maps on the signature. If a pathway gene maps, it increases (e.g., adds one) and when no pathway gene maps it decreases (e.g., subtracts one). Such an approach can constitute run-sum statistics, which leads to NES and p-value calculations.

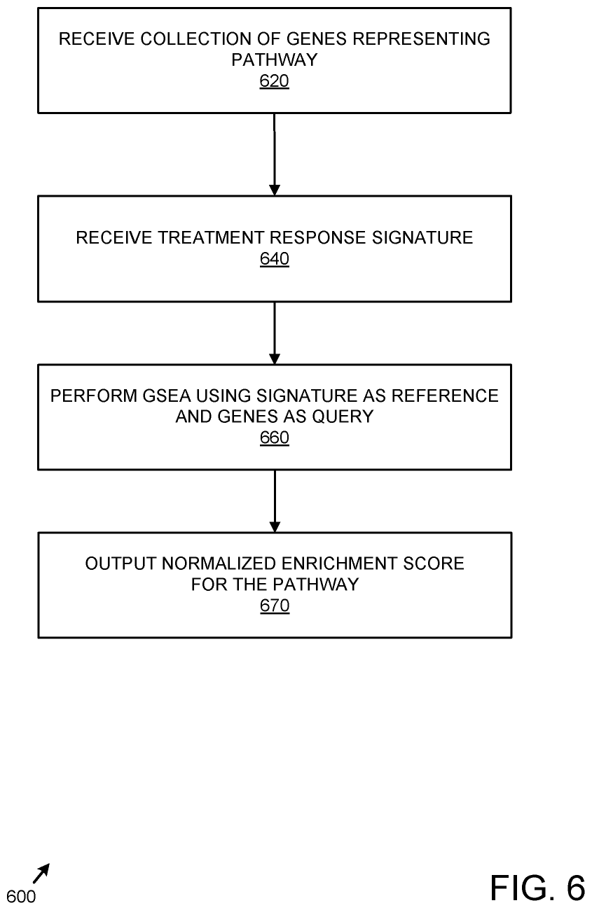

FIG. 6 is a flowchart of an example method 600 calculating transcriptomic and epigenomic pathway signatures and can be implemented, for example, by a system such as that shown in FIG. 5.

At 620, a collection of genes representing a pathway is received.

At 640, a treatment response signature is received.

At 660, GSEA is performed using the signature as a reference and genes as a query.

At 670, the normalized enrichment score is output for the pathway.

In practice, one iteration can be performed for one pathway (e.g., n iterations for n pathways).

### Example 17—Example Composite Signature Generation

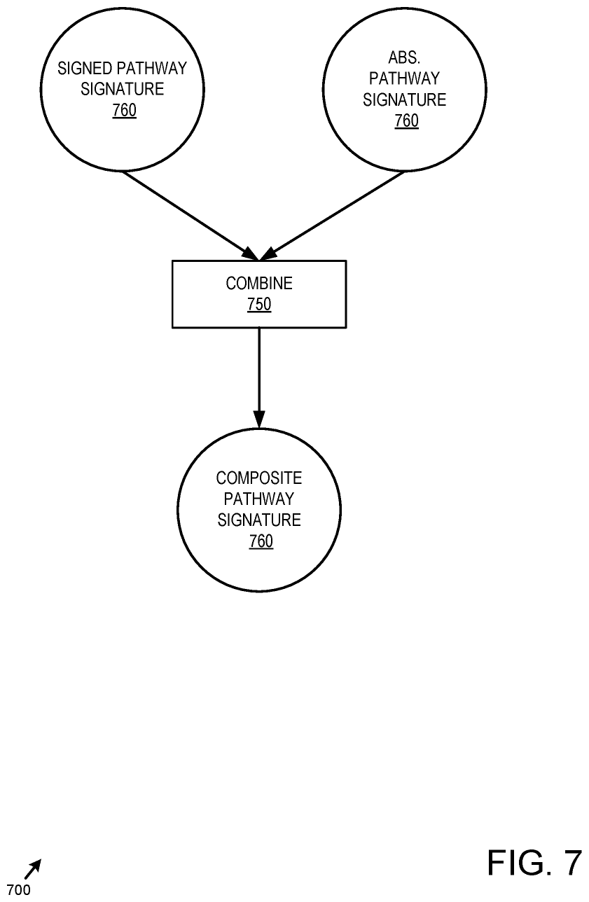

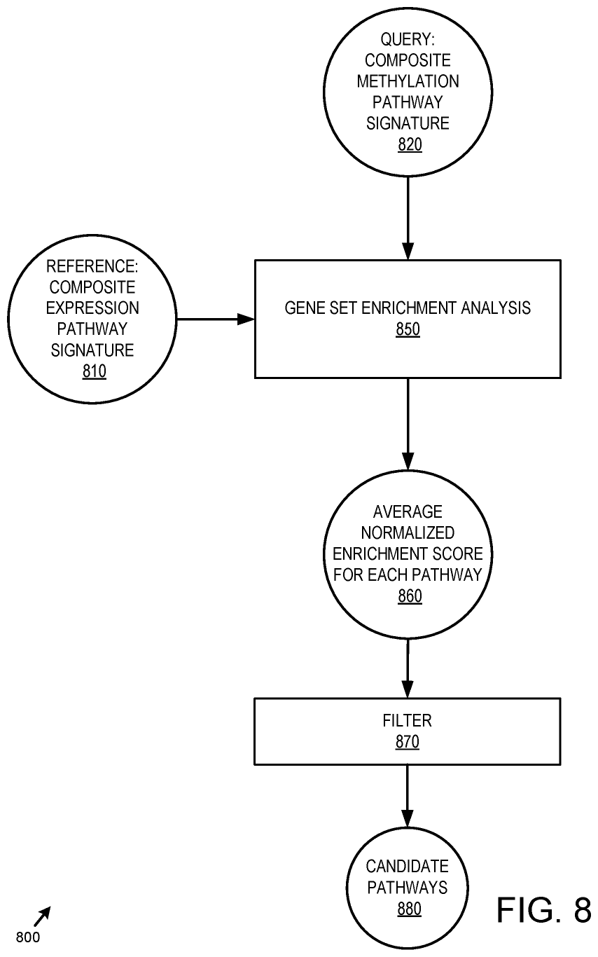

FIG. 7 is a block diagram of an example system 700 calculating a composite pathway signature that can be used in any of the examples herein.

In the example, a signed pathway signature 760 and an absolute values pathway signature 760 are combined 750 into a composite pathway signature 760.

The analysis can be performed twice. For example, once to generate the transcriptomic signature and once to generate the epigenomic pathway signatures (e.g., based on expression and methylation respectively).

The pathways can have their normalized enrichment scores (NES) and p-values in their signed pathway signatures and in the absolute values pathway signatures.

When listing a pathway, one can choose the NES from the signature (either signed or absolute valued pathway signature) which lists the lowest (most significant) p-value for that pathway. For example, if for pathway X, signed signature has NES 5.5 and p-value 0.003 and absolute valued signature has NES 3.2 and p-value 0.04, then one can select NES=5.5 as 0.003 is a more significant p-value. Other techniques can be used.

### Example 18—Example Integrative Pathway Analysis

FIG. 8 is a block diagram of an example system 800 implementing integrative pathway analysis (e.g., that comprises selecting candidate pathways) that can be used in any of the examples herein.

In the example, GSEA 850 receives a reference 810 comprising a composite expression pathway signature and a query 820 comprising a composite methylation pathway signature and outputs average normalized enrichment scores 860 for the pathways.

As shown, the technologies can perform pathway-on-pathway GSEA. As such, one pathway signature can be used a reference signature, and a significant tail of the other pathway signature can be used as a query. So, one pathway signature ranked by its normalized enrichment scores can be used as a reference and significant pathways from another pathway signature can be used as a query.

A filter 870 selects the candidate pathways 880 as described herein.

### Example 19—Example Alternative GSEA Inputs

In any of the examples herein, the query and reference can be switched as inputs when performing GSEA while still achieving useful results. For example, a composite methylation pathway signature can be used as a reference, and a composite expression pathway signature can be used as a query.

### Example 20—Example Overall Method

A computer-implemented method of identifying treatment-response biomarkers can comprise the following:

Input genomic and epigenomic data can be received. For example, genomic and epigenomic datasets can be received for at least two cohorts, wherein the at least two cohorts have different treatment-response phenotypes. In per-patient analysis, a cohort can comprise a single subject or patient.

Differential genomic and epigenomic datapoints can be identified in the data, wherein the identifying generates at least one differential genomic signature for the treatment-response phenotypes and at least one differential epigenomic signature for the treatment-response phenotypes.

Determining biological pathways enriched in the at least one differential genomic signature and the at least one differential epigenomic signature can be determined, wherein the determining generates at least one genomic pathway signature and at least one epigenomic pathway signature.

Biological pathways enriched between the at least one genomic pathway signature and the at least one epigenomic pathway signature can be selected, wherein the selecting generates a comprehensive pathway signature for the treatment-response phenotypes.

The selected biological pathways (e.g. sets of gene identifiers for genes in the pathways) can then be output.

As described herein, gene set enrichment analysis can be performed using various input data structures as queries and references. For example, generating the at least one genomic pathway signature can comprise, for a given input pathway, performing gene set enrichment analysis with genes in the given input pathway as a query and a treatment response signature as a reference.

Also selecting biological pathways enriched can comprise performing, for a given analyzed pathway, pathway-on-pathway gene set enrichment analysis with a composite methylation pathway signature for the given analyzed pathway and a composite expression pathway signature for the given analyzed pathway as a query and a reference.

### Example 21—Example Implementation of Receiving Genomic and Epigenomic Data

Any of the examples herein can include receiving a variety of genomic and epigenomic data (for example, one or more datasets that include one or more datapoints). In practice, genomic and epigenomic data can include genomic and epigenomic data on genes or sets of genes. For example, a targeted set of genes or a genome-wide set of genes can be included.

In practice, receiving genomic and epigenomic data can include genomic and epigenomic data for at least one subject (such as a subject with a known response to treatment, or a training subject, or a subject with an unknown response to treatment, or a query subject) or at least one group of subjects (such a group of subjects with a common feature or characteristic, or a cohort). In specific, non-limiting examples, receiving genomic and epigenomic data can include genomic and epigenomic data for at least 2 cohorts, such as cohorts with a different disease status or with different phenotypes (for example, 2 cohorts with the same disease but different treatment-response phenotypes). For example, FIG. 2 shows receiving 210 a poor response data set and receiving 220 a favorable response data set. In examples, receiving genomic and epigenomic data can include genomic and epigenomic data for a subject or subjects with a common feature or characteristic, such as a disease (for example, cancer, or a malignant tumor characterized by abnormal or uncontrolled cell growth, such as, reproductive, gastrointestinal, pancreatic, blood, kidney, bladder, skin, head and neck, nervous system, bone and cartilage, lymphatic, lung, or colorectal cancer, for example, lung adenocarcinoma, lung squamous cell carcinoma, or colon adenocarcinoma) and/or a treatment-response phenotype (for example, chemotherapy treatment response, such as a response to a chemical or biological agent with therapeutic usefulness in the treatment of diseases characterized by abnormal cell growth, for example, carboplatin, paclitaxel, cisplatin, vinorelbine, folinic acid, fluorouracil, oxaliplatin, or a combination thereof).

In specific, non-limiting examples, receiving genomic and epigenomic data can include genomic and epigenomic data for single subjects or a group of subjects with a common disease (such as cancer, for example, a malignant tumor characterized by abnormal or uncontrolled cell growth, such as reproductive, gastrointestinal, pancreatic, blood, kidney, bladder, skin, head and neck, nervous system, bone and cartilage, lymphatic, lung, or colorectal cancer, for example, lung adenocarcinoma, lung squamous cell carcinoma, or colon adenocarcinoma).

In practice, receiving genomic and epigenomic data can include a variety of processing steps. In examples, processing steps can include normalization, transformation (such as stabilized variance, β value or M value transformation, log transformation, z-score, or rank transformation), redundancy reduction (such as a by selecting a single site one methylation site per gene, such as one CpG site per gene, for example, based on statistical factor, such as a highest coefficient of variation), centering, standardization, logit transformation, bias correction, background correction, and the like.

### Example 22—Example Implementation of Identifying Differential Genomic and Epigenomic Datapoints

Any of the examples herein can include identifying differential genomic and/or epigenomic data (for example, differential genomic and/or epigenomic datapoints in a dataset), such as by a differential identifier. In practice, one or more differential genomic and/or epigenomic signatures can be generated. For example, FIG. 2 shows generating 230 gene expression and DNA methylation signatures.

In examples, differential genomic data or datapoints can include differential expression of genes or sets of genes. For example, genes in which an amount of one or more of its expression products (for example, transcripts, such as mRNA, and/or protein) is higher or lower in one sample (such as a test sample) as compared to another sample (such as a control sample or a reference standard, for example, a healthy subject or subjects or a subject or subjects with a disease and/or treatment response phenotype, such as a subject or subjects who respond positively to chemotherapy, or a subject or subjects who do not develop resistance to chemotherapy, such as a subject who does not respond positively to chemotherapy, such as a subject or subjects who develop resistance to chemotherapy, or a historical control or standard reference value or range of values). In practice, differential expression can include an increase or a decrease in expression of a gene or genes. Differential expression can include a quantitative increase or a decrease in expression, for example, a statistically significant increase or decrease.

In examples, differential epigenomic data or datapoints can include differential methylation of genes or sets of genes. For example, differential methylation can include nucleotides in a gene (such as the gene body) or sequences associated with gene transcription (such as promoters, for example, in CpG islands of promoters) in which methylation is higher or lower in one sample (such as a test sample) as compared to another sample (such as a control sample or a reference standard, for example, a healthy subject or subjects or a subject or subjects with a disease and/or treatment response phenotype, such as a subject or subjects who respond positively to chemotherapy, or a subject or subjects who do not develop resistance to chemotherapy, such as a subject who does not respond positively to chemotherapy, such as a subject or subjects who develop resistance to chemotherapy, or a historical control or standard reference value or range of values). In practice, differential methylation can include an increase or a decrease in methylation of DNA for a gene or genes. Differential methylation can include a quantitative increase or a decrease in methylation, for example a statistically significant increase or decrease.

In examples, various methods can be used to identify differential genes for differential genomic signatures and differential epigenomic signatures. For example, genomic or epigenomic data (such as described herein) for a gene or a set of genes can be compared.

In practice, a variety of processing steps can also be applied. For example, processing can include a quantitative comparison. For example, a statistical comparison can be used, such as a t-statistic (for example, using a two-tailed t-test, such as a Student's or Welch's t-test, for example, a two-tailed Welch's t-test) or other statistical comparison, such as a Wilcoxon-Mann-Whitney test. Thus, genes or a set of genes associated with level of gene expression or epigenomic activity (such as methylation) as described herein can be input into a differential identifier, and a list of genes or set of genes, in which each gene is associated with a level of differential expression or differential epigenomic activity (such as differential methylation) can be output, such as a differential genomic or epigenomic signature, respectively.

In practice, differential genomic signatures and differential epigenomic signatures can be output with a variety of forms. For example, a ranked list (such as based on level of differentiation), a list of genes with significance assigned, or a list of genes that meet an applied cut-off threshold (such as based on level of differentiation. Other forms are possible. For example, where gene differentiation is quantified (for example, producing positive values for overexpression or over-methylation and producing negative values for underexpression or under-methylation), differential genomic signatures and differential epigenomic signatures can include absolute valued differential genomic signatures and differential epigenomic signatures or signed differential genomic signatures and differential epigenomic signatures.

In any of the examples herein, a variety of differential genomic and epigenomic signatures can be generated for genes or a set of genes. In practice, one or more than one differential genomic and epigenomic signature can be generated for genes or a set of genes. In examples, more than one differential genomic and epigenomic signature can be generated for more than one list of genes or a set of genes, such as during training. In examples, a single sample genomic and epigenomic signature can be generated for a single list of genes or a set of genes, such as during use or validation.

In practice, differential genomic signatures and differential epigenomic signatures can include various genes or sets of genes. For example, a targeted set of genes (such as for use or validation, for example, genes associated with pathways in a comprehensive pathway signature) or a genome-wide set of genes can be included (such as for training, for example, using gene or gene sets of biological pathways, such as included in general or specific biological pathways databases, for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like, such as described in Garcia-Campos et al., Front. Physiol, 6(383), 2015, incorporated herein by reference in its entirety).

### Example 23—Example Implementation of Determining Biological Pathways Enriched Differential Genomic Signatures

Any of the examples herein can include determining biological pathways enriched in a differential genomic or epigenomic signature, such as by a pathway enrichment identifier. In practice, one or more genomic or epigenomic signatures can be generated. For example, FIG. 2 shows performing 250A pathway enrichment and combination on the genomic level.

In practice, biological pathways enriched in a differential genomic or epigenomic signature can be determined in a variety of ways. For example, genes or a set of genes in a differential genomic or epigenomic signature can be compared with genes in biological pathways, such as included in general or specific biological pathways databases, for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like (for example, as described in Garcia-Campos et al., Front. Physiol, 6(383), 2015, incorporated herein by reference in its entirety).

In practice, a variety of processing steps can also be applied. For example, processing can include a quantitative comparison. In examples, a statistical comparison can be used, such as the Kolmogorov-Smirnov statistic, Mann-Whitney test, t-tests (for example, Welch's or Student's t-test), chi-square, Fisher's exact test, binomial, probability, hypergeometric distribution, z-score, permutation analysis, kappa statistics and the like. Other enrichment analysis tools or algorithms can be used, such as singular, gene set, or modular enrichment analysis. In specific, non-limiting examples, gene set enrichment analysis can be used (such as with a differential genomic or epigenomic signatures that include genes or gene sets that are ranked based on level of differential expression or methylation), for example, gene set enrichment analysis (GSEA), ErmineJ, FatiScan, MEGO, PAGE, MetaGF, Go-Mapper, ADGO, or the like (such as described in Huang et al., Nucleic Acids Res. 37(1): 1-13, 2009, incorporated herein by reference in its entirety).

In practice, output genomic and epigenomic pathway signatures can take a variety of forms. For example, genomic and epigenomic pathway signatures can include a list of pathways enriched in differential genomic signatures and differential epigenomic signatures. In practice, the list of pathways can include a variety of possible pathways. In examples, possible pathways can include the pathways listed in one or more general or specific pathway databases ((for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like, such as described in Garcia-Campos et al., Front. Physiol, 6(383), 2015, incorporated herein by reference in its entirety), such as during training. In examples, possible pathways can include pathways listed in a comprehensive pathway signature (such as comprehensive pathway signatures disclosed herein), such as during use or validation, for example, in single sample genomic and epigenomic pathway signatures or single sample composite genomic and epigenomic pathway signatures.

In examples, enriched pathways can be quantified based on the level of enrichment in differential genomic signatures and differential epigenomic signatures. For example, an enrichment score (such as a normalized enrichment score) or a p value can be associated with the enriched pathways in the genomic and epigenomic pathway signature output. Other forms are possible, for example, quantified gene expression and methylation levels of the genes in the enriched pathways can be the output.

In examples, output genomic and epigenomic pathway signatures can be generated based on absolute valued differential genomic signatures and differential epigenomic signatures or signed differential genomic signatures and differential epigenomic signatures. Thus, genomic and epigenomic pathway signature output can also include absolute valued genomic pathway signatures and epigenomic pathway signatures or signed genomic pathway signatures and epigenomic pathway signatures. Single sample genomic and epigenomic pathway signature output can also be signed or absolute valued.

### Example 24—Example Implementation of Combining Genomic or Epigenomic Pathway Signatures

Any of the examples herein can include combining genomic or epigenomic pathway signatures (for example, combining a signed genomic pathway signature with an absolute valued genomic pathway signature or combining a signed epigenomic pathway signature with an absolute valued epigenomic pathway signature), such as by a pathway signature combiner. In practice, one or more composite genomic or epigenomic pathway signatures can be generated. For example, FIG. 2 shows performing 250B pathway enrichment and combination on the epigenomic level.

In practice, genomic or epigenomic pathway signatures can be combined in a variety of ways. For example, pathways or quantitation thereof can be compared among one or more signatures. In examples, normalized enrichment scores or p values can be compared. For example, normalized enrichment scores or p values can be compared for overlapping pathways (or the same pathway, such as the same pathways listed in a signed genomic pathway signature and an absolute valued genomic pathway signature or the same pathways listed in a signed epigenomic pathway signature and an absolute valued epigenomic pathway signature), and a highest normalized enrichment score or lowest p value for the same pathway among pathway signatures can be selected for inclusion in a combined or composite pathway signature.

In practice, output composite genomic and epigenomic pathway signatures can include all or some information from multiple genomic or epigenomic pathway signatures. In examples, multiple genomic and epigenomic pathway signatures can include absolute valued genomic pathway signatures and signed genomic pathway signatures in composite genomic pathway signatures or absolute valued epigenomic pathway signatures and signed epigenomic pathway signatures in composite epigenomic pathway signatures.

In practice, composite genomic or epigenomic pathway signatures can be output in a variety of ways. For example, output composite genomic or epigenomic pathway signatures can include a list of enriched pathways that have been quantified, wherein a highest level of enrichment for each pathway among the combined pathway signatures can be associated with each pathway in the composite genomic and epigenomic pathway signatures.

### Example 25—Example Implementation of Selecting Pathways Enriched Between Pathway Signatures

Any of the examples herein can include selecting biological pathways enriched between genomic pathway signatures and epigenomic pathway signatures, such as by a comprehensive enriched pathway identifier. In practice, one or more comprehensive pathway signatures can be generated. For example, FIG. 2 shows integrating 260 composite pathway signatures, yielding candidate pathways (e.g., selected or discovered pathways).

In practice, biological pathways enriched in between genomic pathway signatures and epigenomic pathway signatures can be determined in a variety of ways. For example, biological pathways in genomic pathway signatures can be compared with genes in biological pathways.

In practice, a variety of processing steps can also be applied. For example, processing can include a quantitative comparison. For example, a statistical comparison can be used, such as the Kolmogorov-Smirnov statistic, Mann-Whitney test, t-tests (for example, Welch's or Student's t-test), chi-square, Fisher's exact test, binomial, probability, hypergeometric distribution, z-score, permutation analysis, kappa statistics and the like. Other enrichment analysis tools or algorithms can be used, such as singular, gene set, or modular enrichment analysis. In specific, non-limiting examples, gene set enrichment analysis can be used (such as with a differential genomic or epigenomic signatures that include genes or gene sets that are ranked based on level of differential expression or methylation), for example, gene set enrichment analysis (GSEA), ErmineJ, FatiScan, MEGO, PAGE, MetaGF, Go-Mapper, ADGO, or the like (such as described in Huang et al., Nucleic Acids Res. 37(1): 1-13, 2009, incorporated herein by reference in its entirety).

In practice, output comprehensive pathway signatures can take a variety of forms. For example, comprehensive pathway signatures can include a list of pathways enriched in genomic pathway signatures and epigenomic pathway signatures.

In examples, enriched pathways can be quantified based on the level of enrichment in genomic pathway signatures and epigenomic pathway signatures. For example, an enrichment score (such as a normalized enrichment score) or a p value can be associated with the enriched pathways in the comprehensive pathway signature output. Other forms are possible.

In practice, output comprehensive pathway signatures can include pathways with certain features or characteristics, such as pathways enriched at least two biological phenomena or pathways with statistical significance. Output comprehensive pathways can also exclude pathways with certain features or characteristics, such as redundant pathways (such as pathways with more than one convention, for example, pathways that are included in both narrowly and broadly defined conventions).

### Example 26—Example Implementation

Disclosed herein are systems and methods to uncover interplay between genomic and epigenomic mechanisms and elucidate the complexity of the chemotherapy response in cancer patients. These systems and methods integrate genomic information (such as mRNA expression) and epigenomic information (such as DNA methylation) from patient profiles to identify molecular pathways with significant alterations on genomic and epigenomic levels to distinguish favorable from poor chemotherapy treatment responses.

The systems and methods disclosed herein were used on patients with lung adenocarcinoma who received a carboplatin and paclitaxel combination chemotherapy (carboplatin-paclitaxel), a standard-of-care for treating advanced lung cancer. This integrative approach identified seven molecular pathways with significant epigenomic alterations that distinguish favorable from poor carboplatin-paclitaxel response, including chemokine receptors, mRNA splicing, G alpha signaling events, and immune network for IgA production. These pathways can be used to classify patients based on their risk of developing carboplatin-paclitaxel resistance in an independent patient cohort (log-rank p-value=0.0081), and their predictive ability is independent of and not affected by (i) signatures of overall lung cancer aggressiveness or (ii) commonly utilized covariates, such as age, gender, and stage at diagnosis (adjusted hazard ratio=14.0). Demonstrating the generalizability of these systems and methods, they were applied across additional chemotherapy regimens (i.e., cisplatin-vinorelbine, oxaliplatin-fluorouracil) and cancer types (i.e., lung squamous cell carcinoma and colorectal adenocarcinoma), showing their ability to accurately predict patients' treatment response.

Thus, the systems and methods herein can be utilized to identify epigenomically altered pathways implicated in primary chemoresponse and effectively classify patients who would benefit from specific chemotherapy regimens or are at risk of resistance, significantly improving personalized therapeutic strategies and informed clinical decision making.

### Example 27—Example Schematic Representation

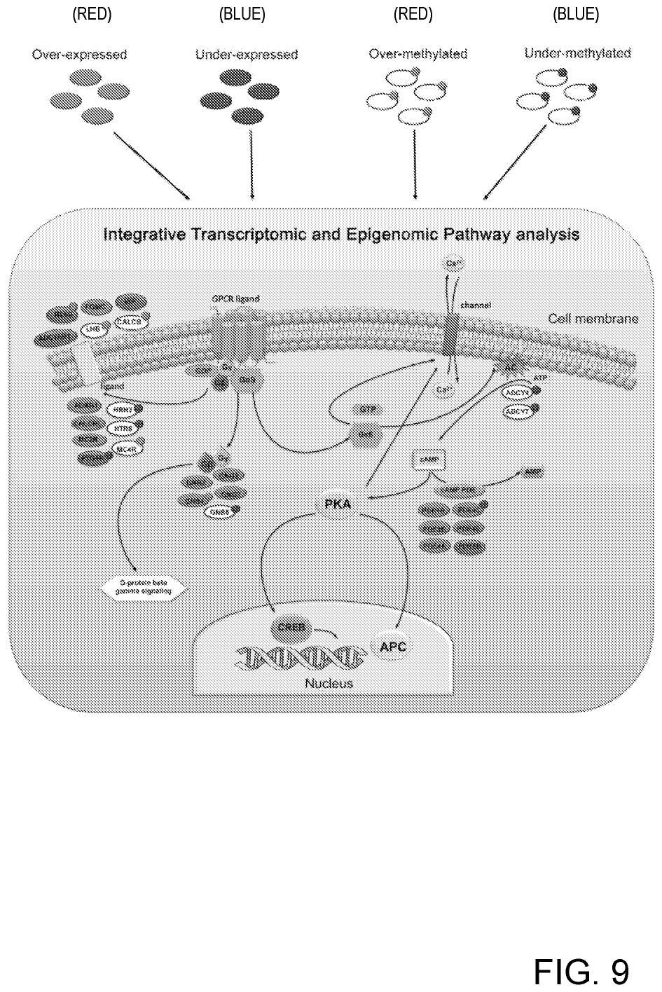

FIG. 9 shows a schematic representation of an example pathway altered at both genomic and epigenomic levels. Pathway genes affected on genomic and epigenomic levels in G alpha signaling events pathway are represented by ovals, and the colors correspond to either over-expression (red), under-expression (blue), or no differential expression (white). Small satellite circles represent over-methylation (red) or under-methylation (blue).

### Example 28—Example Implementation

Lung adenocarcinoma patient cohorts: LUAD patient cohorts were obtained from publicly available data sources, which include The Cancer Genome Atlas-Lung Adenocarcinoma (TCGA-LUAD) (Cancer Genome Atlas Research Network, Nature. 2014; 511(7511):543-50), Tang et al. (GSE42127) (Tang et al., Clin. Can. Res. 2013; 19(6):1577-86), Der et al. (GSE50081) (Der et al., J. Thor. Oncol. 2014; 9(1):59-64), and Zhu et al. (GSE14814) (Zhu et al., J. Clin. Oncol. 2010; 28(29):4417-24) datasets (Example 20). The primary LUAD patient cohort that was utilized for reconstruction of epigenomic signatures of chemoresistance was obtained from The Cancer Genome Atlas (TCGA-LUAD) project (Cancer Genome Atlas Research Network, Nature. 2014; 511(7511):543-50) and downloaded from the Genomics Data Commons database (GDC; portal.gdc.cancer.gov) on February 2017. Clinical information (such as clinical files, follow-ups, and treatment data) for these datasets were obtained from the TCGA GDC legacy archive (portal.gdc.cancer.gov).

To study primary resistance to the carboplatin-paclitaxel combination (Example 20) in LUAD, the patients selected had primary tumors obtained at surgery (n=14) and did not receive neo-adjuvant treatment (no therapy prior to sample collection) but were treated with an adjuvant carboplatin (platinum-based alkylating chemotherapy) and paclitaxel (non-platinum based plant alkaloid chemotherapy taxane) combination. These patients were further monitored for disease progression; disease progression was defined as a new tumor event, including tumor re-occurrence, and local and distant metastases. TCGA-LUAD mRNA expression (RNA seq) data were profiled using an Illumina HiSeq 2000, and DNA methylation was profiled using an Illumina Infinium Human Methylation (HM450) array. For validation studies, the Tang et al. (Tang et al., Clin. Can. Res. 2013; 19(6):1577-86) (GSE42127) cohort was used, which captures primary LUAD tumors obtained at surgery (n=39) and that were not pre-treated (no neoadjuvant treatment) but treated with an adjuvant carboplatin and taxane (paclitaxel) chemotherapy combination and profiled on an Illumina HumanWG-6 v3.0 expression beadchip. Cohorts used for negative controls included (i) the Der et al. (Der et al., J. Thor. Oncol. 2014; 9(1):59-64) (GSE50081) patient cohort with LUAD that never received treatment (n=127), which was profiled using an Affymetrix Human Genome U133 Plus 2.0 Array; (ii) Tang et al. (Tang et al., Clin. Can. Res. 2013; 19(6):1577-86) (GSE42127) patient cohort with LUAD that did not receive any treatment (n=94), profiled on Illumina HumanWG-6 v3.0 expression beadchip (Example 20).

Signatures of LUAD aggressiveness were obtained from: (i) Larsen et al. (Larsen et al., Clin. Can. Res. 2007; 13(10):2946-54), which identified 54 prognostic LUAD markers; (ii) Beer et al. (Beer et al., Nature medicine. 2002; 8(8):816-24), which identified 50 prognostic LUAD markers; and (iii) Tang et al. (Tang et al., Clin. Can. Res. 2013; 19(6):1577-86), which identified 12 prognostic non-small cell lung cancer markers (non-small cell lung cancer is a class of lung cancer, which includes LUAD).

Gene expression and DNA methylation analysis: For the RNA-seq analysis, the variance for raw RNA-seq counts was normalized and stabilized using the DESeq2 (Love et al., Genome Biology. 2014; 15(12):550) R package. DNA methylation values for each site were reported as β (Beta) values, which were subsequently converted to M-values (Du et al., BMC Bioinformatics. 2010; 11(1):587) for statistical analysis, using the beta2m function in the Lumi (Du et al., Bioinformatics. 2008; 24(13):1547-8) R package. To avoid redundancy introduced by multiple sites present for each gene, one CpG site was selected per gene through the coefficient of variation analysis, where a site with the highest coefficient of variation was selected for each gene.

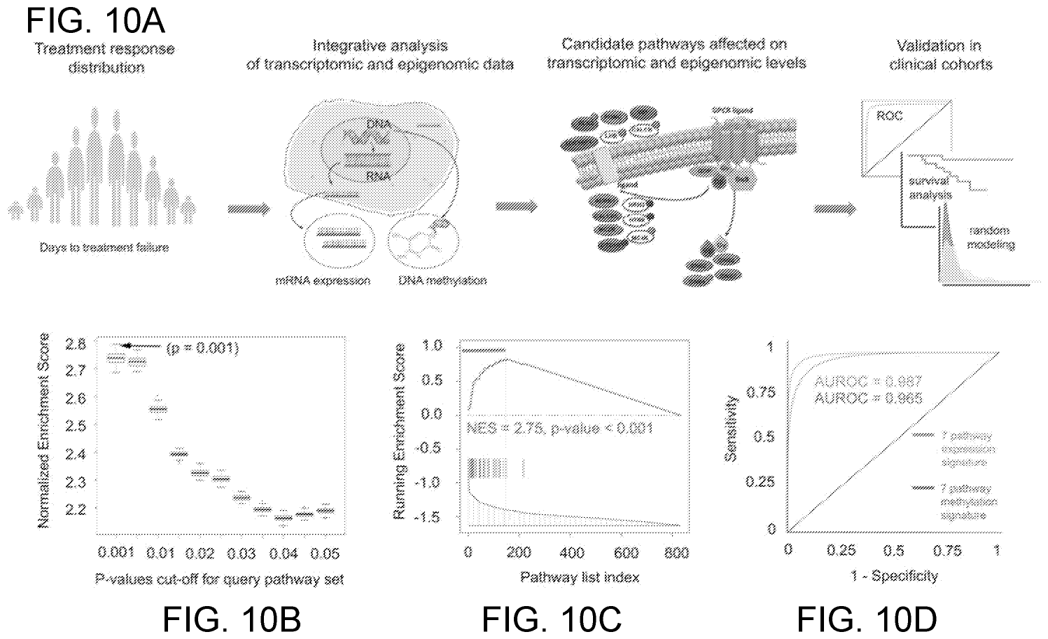

Defining signatures of chemotherapy response: The next step in the analysis was to define a signature of response to carboplatin-paclitaxel combination. For this, clinical data was analyzed from 14 patients that received carboplatin-paclitaxel chemo treatment in the TCGA-LUAD patient cohort (Example 20). To identify patients that failed the treatment and patients with a favorable response, the time between carboplatin-paclitaxel start and disease progression (a new tumor event was defined as tumor reappearance or local or distant metastases) or latest follow-up was analyzed for each patient. Next, a failed/poor treatment response was defined as patients whose disease progressed within 1 year of treatment start and a favorable response was defined as patients who stayed disease progression-free for over 2 years. To ensure that patients were not biased by initial tumor aggressiveness, local or distant metastatic burden, age, or smoking status, patients from each group were selected with similar distributions for (i) age, (ii) gender, (iii) tumor stage at diagnosis, and (iv) smoking status (Table 1, related to FIGS. 10, 11, 16, 17, 18, and 21), which defined feature-comparable groups of 4 poor-response and 4 favorable-response patients, utilized for further analysis.

TABLE 1
Clinical profiles of carboplatin-paclitaxel treated patients with poor (n =
4) and favorable (n = 4) treatment response from the TCGA-LUAD cohort.

| | | Time to | | | | | | Observed |
|---|---|---|---|---|---|---|---|---|
| | | event or | | | Tumor | | # | treatment |
| Treatment | Patient | follow-up | | | stage at | Smoking | pack | related event |
| response | ID | (days) | Age | Gender | diagnosis | status | years | or follow-up |
| poor | 6712 | 116 | 71 | male | IIA | 4 | NA | new tumor event |
| response | 5051 | 122 | 42 | female | IIIA | 4 | 30 | new tumor event |
| | 6979 | 138 | 59 | female | IIB | 3 | NA | new tumor event |
| | A4VP | 153 | 66 | female | IIIA | 4 | 20 | new tumor event |
| favorable | 4666 | 744 | 52 | female | IV | 4 | 10 | no event, follow-up |
| response | 5899 | 784 | 58 | male | IIA | 2 | NA | no event, follow-up |
| | 1678 | 1120 | 70 | female | IIB | 3 | 20 | no event, follow-up |
| | 1596 | 2031 | 55 | male | IIB | 2 | 50 | no event, follow-up |

Notes:
NA = not available.
Smoking status: 1 = lifelong non-smoker (<100 cigarettes smoked in Lifetime), 2 = current smoker (includes daily smokers and non-daily smokers (or occasional smokers), 3 = current reformed smoker for >15 years, 4 = current reformed smoker for ≤15 years, 5 = current reformed smoker, duration not specified, and 6 = smoking history not documented.

To determine the molecular characteristics that differ between poor response and favorable response, signatures of treatment response were defined at the genomic level (for example, differential expression) and epigenomic (for example, differential methylation) level between poor-response and favorable-response patient groups using the two-sample two-tailed Welch t-test (t.test function in R) (Welch, Biometrika. 1947; 34(1-2):28-35) in R studio version 3.3.2 (Team RC, Foundation for Statistical Computing; 2016. 2017), such that a differential expression signature was defined as a list of genes ranked on their differential expression (t-test values), and the differential methylation signature was defined as a list of genes based on the differential methylation of the corresponding site (t-test values).

Genomic and epigenomic pathway enrichment analysis: To identify molecular pathways significantly altered at the genomic and epigenomic levels (for example, FIG. 9), a pathway enrichment analysis was performed for a differential expression signature and differential methylation signature (for example, FIG. 15). For this analysis, the comprehensive C2 pathway database was used (Liberzon et al., Bioinformatics. 2011; 27(12):1739-40) (software.broadinstitute.org), which includes 833 pathways from the REACTOME (Fabregat et al., Nucleic Acids Res. 2016; 44(D1):D481-7), KEGG (Ogata et al., Nucleic Acids Res. 1999; 27(1):29-34), and BIOCARTA (Nishimura D., Biotech Software & Internet Report: The Computer Software Journal for Scient. 2001; 2(3):117-20) databases, and a pathway enrichment analysis was implemented using the Gene Set Enrichment Analysis (GSEA) (Subramanian et al., PNAS. 2005(102):15545-50), in which differential expression and differential methylation signatures were used as reference and collection of genes from each pathway was used as a query gene set. Normalized Enrichment Scores (NESs) and p-values were estimated using 1,000 gene permutations. This analysis estimated NESs for each of the 833 pathways, which reflects how much each pathway is enriched in the treatment response signature and defines a so-called pathway activity. A positive NES reflects pathway enrichment in the over-expressed part of the signature (a majority of pathway genes are over-expressed) and negative NES reflects pathway enrichment in the under-expressed part of the signature (a majority of pathway genes are under-expressed). Such pathway enrichment analysis is referred to as “signed” because it considers over- and under-expression of genes (with direction).

Further, to overcome limitations of such (signed) pathway enrichment analysis, which assumes that the pathway will be enriched only if majority of genes in the pathway are changed in the same direction (such as over-expressed or under-expressed, but not both), an “absolute valued” analysis was performed. For this, the pathway enrichment analysis was performed using the “absolute valued” differential expression signature, in which signature t-stat values was absolute valued to “collapse” positive and negative signature tails, as was previously performed in (Dutta et al., European Urology. 2017; 72(4):499-506). In this case, positive NESs reflect enrichment in a part of the signature with significant differential expression (including both over-expressed and under-expressed genes), and negative NESs reflect enrichment in the non-differentially expressed part of the signature (and are therefore not considered as significant). This absolute valued pathway enrichment analysis yields pathways with genes that might be changed in both directions (both over-expressed and under-expressed) because it estimates enrichment in the differentially expressed tail of the signature (irrespective of sign). Such absolute valued pathway enrichment analysis provided NESs for each of 833 pathways, as described above. An “absolute valued” pathway enrichment analysis was performed using the differential methylation signature of treatment response in the similar manner.

The next step was to then integrate NESs from signed and absolute valued pathway enrichment analysis such that, for each pathway, a final integrative NES was defined as an NES with the most significant p-value between the signed and absolute valued pathway analysis (negative NES values for absolute valued analysis were not considered because they reflect enrichment in the non-changed part of the signature). The advantage of such an integration is two-fold: it captures (1) pathways with genes that are strictly over-expressed or under-expressed in each pathway and (2) pathways with genes that are significantly changed in both directions (i.e., pathways that include genes that are significantly over-expressed and genes that are significantly under-expressed). Thus, the integration increases the probability of identifying functionally relevant molecular determinants. Such an integration of signed and absolute valued NESs provides a composite expression pathway signature and a composite methylation pathway signature.

Genomic and epigenomic pathway integration: To identify pathways that are significantly affected on both genomic and epigenomic levels, GSEA was employed to compare composite expression pathway signatures and composite methylation pathway signatures to identify pathways that are significantly affected on both genomic and epigenomic levels (pathways that belong to the leading edge of the GSEA analysis). To ensure identification of pathways that are (i) over-expressed and under-methylated, (ii) under-expressed and over-methylated, and (iii) differentially expressed and differentially methylated, each pathway signature was ranked based on the absolute values of their NESs and used for a subsequent GSEA comparative analysis.

For this pathway-based GSEA, a composite expression pathway signature was used as a reference signature, and top pathways from the composite methylation pathway signature were used as a query pathway set. To accurately define a query pathway set that ensures the most significant enrichment between pathway signatures, the threshold for the query pathway set was varied between 0.001 and 0.05 (width of each step=0.005), and the strength of enrichment between the two signatures was estimated at each threshold. For each threshold, GSEA was run 100 times, and the average NES for the enrichment was reported. The threshold with the highest average NES then reflects the optimal threshold that corresponds to the most significant enrichment between the composite expression pathway signature and the composite methylation pathway signature and was used for subsequent analysis. GSEA analyses between the composite expression pathway signature and the composite methylation pathway signature at the optimal threshold identified a set of 28 pathways of treatment response, which were significantly altered on both genomic and epigenomic levels.

One of the limitations of the pathways from the C2 collection is that they often represent a parent-child relationship, where a parent pathway (such as a cell cycle) would encompass all genes in child pathways (such as cell cycle phase). Such overlap produces data redundancy and can result in model overfitting as the “same” pathways are fit in the model repeatedly. To overcome this limitation and to eliminate pathways with heavy overlaps, a Fisher Exact Test (Fisher R A, Journal of the Royal Statistical Society. 1922; 85(1):87-94) (fisher.test function in R) was performed, and leading edge genes for each pair of pathways from the analysis were compared (for all 28 pathways, which resulted in [28 choose 2=378] comparisons). From each group of parent-children pathways that shared a large number of overlapping genes, one representative pathway was selected with the most significant NES, which defined a final set of seven (7) maximally non-overlapping non-redundant pathways used for subsequent analysis.

Evaluating expression and methylation data in the integrative analysis: To examine if both data types (mRNA expression and DNA methylation) from the 7 candidate pathways have the equivalent ability to predict a therapeutic response, the performance of the 7 pathways was compared utilizing only their (i) activity levels based on expression and (ii) activity levels based on methylation, separately. To compare pathway performances based on each data type, both expression and methylation data matrices (z-scored on genes) were scaled in the TCGA-LUAD cohort, which defined single-sample differential expression and single-sample differential methylation signatures, respectively. Each sample was then used for signed and absolute valued pathway enrichment analysis (separately for expression and for methylation, as above), in which each single-sample signature was used as a reference, and genes from each of 7 candidate pathways were used as a query set, thus, producing a pathway activity signature for each patient. These single-sample expression and methylation pathway signatures were then used to evaluate the predictive ability of 7 pathways (for expression and methylation, separately) using logistic regression modeling (Walker et al., Biometrika. 1967; 54(1/2):167-79) followed by Receiver Operating Characteristic (ROC) analysis (Metz C E, Seminars in nuclear medicine. 1978; 8(4):283-98). Here, the area under ROC (AUROC) reflected how well each data type separates poor-response and favorable-response patients in the TCGA-LUAD patient cohort (the AUROC value of 0.5 indicates a random predictor, and 1 indicates a perfect predictor). The logistic regression analysis was done using glm (Chambers et al., Statistical Models in S1990; Heidelberg: Physica-Verlag H D) function and ROC analysis was done using pROC (Robin X et al., BMC bioinformatics. 2011; 12(1):77) and ggplot2 (Wickham, J Stat Softw. 2010; 35(1):65-88) package in R.

Validation and robustness in independent clinical cohorts: To evaluate clinical significance of the 7 candidate molecular pathways, their ability to predict patients at risk of chemoresistance was examined in an independent clinical cohort from the Tang et al. (Tang et al., Clin. Can. Res. 2013; 19(6):1577-86) dataset, and survival status was used during the clinical study (1996 to 2007) as a clinical endpoint (time to event or follow-up was estimated between the start of carboplatin-paclitaxel treatment and death or follow-up, respectively; maximum time to event/follow-up is 2,567 days).

First, activity levels of the 7 candidate pathways in the Tang et al. cohort were estimated on a single-sample level, as above. The activity levels (NESs) of the 7 candidate pathways were then subjected to t-Distributed Stochastic Neighbor Embedding (t-SNE) clustering (Maaten Lvd et al., Journal of machine learning research. 2008; 9(Nov.):2579-605) (implemented through Rtsne (Maaten L V D., J Mach Learn Res. 2014; 15(1):3221-45) package in R), a non-linear dimensionality reduction technique which chooses two similarity measures between pairs of points of (i) high dimensional input space and (ii) low-dimensional embedding space. First, it constructs a probability distribution over the pairs of high dimensional space (7-dimension in this case) in such a way that similar points are exhibited by nearby instances, while dissimilar points are exhibited by distant instances. Second, it constructs a similar probability distribution over the points in low-dimensional embedding space and tries to minimize the Kullback-Leibler divergence (KL divergence) (Kullback et al., Ann Math Statist. 1951; 22(1):79-86) between the high dimensional data and low dimensional anticipated data at each point. Therefore, patients with similar pathway activity levels will be anticipated as nearby instances, while patients with dissimilar pathway activity levels will be anticipated as dissimilar instances. The advantage of t-SNE lies in its ability to reduce dimensions from seven (maximum possible in the analysis) to two and effectively identify groups of patients that share similar pathway activity levels. This analysis stratified patients into two groups: a group with overall increased composite pathways' activities and a group with overall decreased composite pathways' activities. Next, whether or not these patient groups significantly differ in their response to carboplatin-paclitaxel treatment was examined using a Kaplan-Meier survival analysis (Kaplan et al., Journal of the American Statistical Association. 1958; 53(282):457-81) and Cox proportional hazards model (Cox D R., Journal of the Royal Statistical Society Series B (Methodological). 1972; 34(2):187-220) via survival (Therneau T., A package for survival analysis in S. R package version 2.38. Retrieved from CRAN R-project org, 2015), ggplot2 (Wickham, J Stat Softw. 2010; 35(1):65-88), and survminer (Kassambara et al., survminer: drawing survival curves using ‘ggplot2’. R package version 0.2. 4. 2016) R packages.

In order to evaluate whether a random set of pathways can perform as well as the identified 7 pathways, the predictive ability of the 7 candidate pathways was compared with the predictive ability of 7 pathways selected at random. For this analysis, a random model was constructed, in which 7 pathways were selected at random, and their activity levels were utilized to stratify patients based on their treatment response with a subsequent evaluation using a Kaplan-Meier survival analysis. Random selection was performed 10,000 times, and the empirical p-value was estimated as the number of times a Kaplan-Meier log-rank p-value for 7 candidate molecular pathways outperformed the results at random. Also employed was a second random model, in which the effect of selecting random patient groups was evaluated.

Finally, to estimate the accuracy with which the systems and methods disclosed herein can predict a treatment response for a new incoming patient, this process was simulated using leave-one-out cross-validation (LOOCV) (Stone M., Journal of the royal statistical society Series B (Methodological). 1974:111-47). In LOOCV, one patient is “removed”, and the model is trained on the rest of the patients. The patient that was removed is considered a new incoming patient, subjected to predictive analysis, and assigned a risk of developing resistance. This process was repeated for all patients. The predictive model for LOOCV was implemented using generalized linear modeling (such as multivariable logistic regression) through the glm (Chambers et al., Statistical Models in S1990; Heidelberg: Physica-Verlag HD) function and ggplot2 (Wickham, J Stat Softw. 2010; 35(1):65-88) package in R.

Comparison to other methods, common covariates, and signatures of aggressiveness: To assess exemplary advantages of the systems and methods disclosed herein, (i) its predictive performance was compared to other commonly utilized approaches, including linear regression modeling, support vector machine, and random forest; and (ii) whether or not the method can be affected by commonly used covariates or known signatures of lung cancer aggressiveness was evaluated.

First, to demonstrate exemplary advantages of the systems and methods disclosed herein over other commonly utilized approaches, performance of these systems and methods was compared with (i) Panja et al. (Panja et al., EBioMedicine. 2018), Epigenomic and Genomic mechanisms of treatment Resistance (Epi2GenR), which uses linear regression to integrate DNA methylation and mRNA expression data; (ii) Zhong et al. (Zhong et al., Scientific reports. 2018; 8(1):12675), which is based on a support vector machine (SVM) algorithm that uses patient mRNA expression profiles; and (iii) Yu et al. (Yu et al., Scientific reports. 2017; 7:43294), Personalized REgimen Selection (PRES) method, which is based on a random forest machine learning approach that uses patient mRNA expression profiles. The selection and cross-validation techniques were followed as suggested in each of the above publications to carefully compare their performance to the systems and methods disclosed herein. Epi2GenR utilized the same signature as utilized in these Examples 1 and 2. To apply SVM and PRES correctly, the validation set was split into 70:30 proportion subsets, in which 70% of the validation set was used for model training, and 30% was used for model validation. The predictive ability of the identified candidates from each method was evaluated using ROC, Kaplan-Meier survival, and hazard ratio analyses through the survival (Therneau T., A package for survival analysis in S. R package version 2.38. Retrieved from CRAN R-project.org, 2015), survcomp (Schroder M S, et al., Bioinformatics 2011; 27(22):3206-8), and survminer (Kassambara et al., survminer: drawing survival curves using ‘ggplot2’. R package version 0.2. 4. 2016) packages in R.

Second, whether any of the commonly used covariates (such as age, gender, and tumor stage at diagnosis) and known signatures of lung cancer aggressiveness (such as from Larsen et al. (Larsen et al., Clin. Can. Res. 2007; 13(10):2946-54), Beer et al. (Beer et al., Nature medicine. 2002; 8(8):816-24), and Tang et al. (Tang et al., Clin. Can. Res. 2013; 19(6):1577-86) described above) can predict a therapeutic response or can significantly affect the predictive ability of the identified 7 candidate pathways was evaluated. For this analysis, the multivariable Cox proportional hazards model (Cox D R., Journal of the Royal Statistical Society Series B (Methodological). 1972; 34(2):187-220) (using coxph function) and stratified Kaplan-Meier survival analysis (Kaplan et al., Journal of the American Statistical Association. 1958; 53(282):457-81) were used through the survival (Therneau T., A package for survival analysis in S. R package version 2.38. Retrieved from CRAN R-project.org, 2015), and survminer (Kassambara et al., survminer: drawing survival curves using ‘ggplot2’. R package version 0.2. 4. 2016) packages in R.

Model generalizability: To test the generalizability of the model, the systems and methods disclosed herein were applied to additional chemotherapy combinations (such as cisplatin-vinorelbine and oxaliplatin-fluorouracil) and additional cancer types (such as lung squamous cell carcinoma and colorectal adenocarcinoma). The investigations included the response to (i) cisplatin (platinum-based alkylating chemotherapy) and vinorelbine (non-platinum based plant alkaloid chemotherapy) response in lung adenocarcinoma (LUAD); (ii) cisplatin-vinorelbine response in lung squamous cell carcinoma (LUSC); and (iii) oxaliplatin (platinum-based alkylating chemotherapy), fluorouracil (antimetabolite chemotherapy), and folinic acid (chemotherapy protective drug often given with fluorouracil to improves the binding; also known as leucovorin) (FOLFOX) response in colorectal adenocarcinoma (COAD).

For signature development, primary tumor samples from TCGA-LUAD/TCGA-LUSC/TCGA-COAD (n=8) were used for patients without neo-adjuvant treatment (no pre-treatment), who received adjuvant chemotherapies of interest and were further monitored for new tumor events (as defined above). As in the TCGA cohorts above, mRNA expression (RNA seq) was profiled using an Illumina HiSeq 2000, and DNA methylation was profiled using an Illumina Infinium Human Methylation (HM450) array.

For clinical validation of the cisplatin-vinorelbine combination response in LUAD, the Zhu et al. patient cohort (Zhu et al., J. Clin. Oncol. 010; 28(29):4417-24) (GSE14814) was used, which included LUAD tumors obtained at surgery (n=39), treated with adjuvant cisplatin-vinorelbine chemotherapy, and profiled on Affymetrix Human Genome U133A platform. In this cohort, lung cancer-related death was used as a clinical endpoint, and time to event was calculated between the start of cisplatin-vinorelbine treatment and lung-cancer related death (for patients with this event) or to follow-up (for censored patients) with the maximum time to event/follow-up at 3,390 days.

For clinical validation of the cisplatin-vinorelbine combination response in lung squamous cell carcinoma (LUSC), a different subset of patients from the Zhu et al. patient cohort (Zhu et al., J. Clin. Oncol. 2010; 28(29):4417-24) (GSE14814) was used, which included patients with LUSC, whose tumors were obtained at surgery (n=26) and who were treated with adjuvant cisplatin-vinorelbine chemotherapy and profiled on Affymetrix Human Genome U133A platform. In this cohort, lung cancer-related death was used as a clinical endpoint, and the time to event was calculated between the start of cisplatin-vinorelbine treatment and lung-cancer related death (for patients with this event) or to follow-up (for censored patients) with the maximum time to event/follow-up at 3,318 days.

Finally, for validation of the FOLFOX combination in colorectal adenocarcinoma (COAD), the Marisa et al. patient cohort (Marisa et al., PLoS medicine. 2013; 10(5):e1001453) (GSE39582) was used, which includes COAD tumors obtained at surgery (n=23), treated with adjuvant FOLFOX chemotherapies, and profiled on Affymetrix Human Genome U133 Plus 2.0 Array. In this cohort, relapse-free survival (where relapse was defined as locoregional or distant recurrence) was used as a clinical endpoint, and time to event was calculated between the start of FOLFOX treatment to relapse (for patients with this event) or to follow-up (for censored patients), with the maximum time to event/follow-up at 2,790 days. The clinical characteristics of subjects are summarized in Example 20.

### Example 29—Example Implementation

Systems and methods for genome-wide computation were developed that can integrate mRNA expression and DNA methylation patient profiles to identify pathways altered at both genomic and epigenomic levels (as demonstrated in FIG. 9) that differentiate poor and favorable responses to chemotherapy regimens. Here, steps included in the integrative systems and methods (also shown in Example 20) are provided. Step 1: two groups of patients are identified, which are used to define a “chemotherapy response signature”: (i) patients that failed a specific chemotherapy regimen (such as patients that developed metastasis within 1 year after therapy administration) and (ii) patients with a favorable chemotherapy response (such as patients that remained disease-free for more than 2 years after chemotherapy administration). Step 2: genomic (mRNA expression) and epigenomic (DNA methylation) profiles are compared between the two groups of patients, which define differential (i) genomic signature and (ii) epigenomic signature of chemoresponse. Step 3: such signatures are individually subjected to signed and absolute valued pathway enrichment analysis, which yields molecular pathways enriched in the genomic signature (composite pathways with genes that are differentially expressed) and pathways enriched in the epigenomic signature (composite pathways with genes are differentially methylated). Step 4: The composite genomic and epigenomic pathway signatures are then integrated to determine a set of pathways that control both genomic and epigenomic programs that are disrupted in resistance. Step 5: candidate pathways are subjected to validation studies, in which they are evaluated for their ability to predict therapeutic response in independent patient cohorts through a multivariable survival analysis. Step 6: finally, the identified pathways are used to assign individual risk of resistance for new incoming patients.

FIG. 9 shows a schematic representation of an example pathway altered at both genomic and epigenomic levels.

FIGS. 10A-10D show an example integrative systematic epigenomic analysis that identifies candidate molecular pathways for a chemotherapy response. FIG. 10A shows an example schematic representation of the integrative epigenomic analysis. From left to right, (left) patients are defined by their response to chemotherapy, (middle left) analysis of genomic and epigenomic patient profiles, (middle right) integrative epigenomic analysis identifies candidate pathways affected on both genomic and epigenomic levels, and (right) multi-modal validation of candidate pathways. FIG. 10B shows an example box and whisker plot depicting p-value cutoff for query carboplatin-paclitaxel response composite methylation pathway signature (x-axis) and NESs from the corresponding GSEA comparison between composite methylation and expression pathways signatures (y-axis), based on analysis in TCGA-LUAD patient cohort. The arrow indicates an optimal p-value threshold, which results in the most significant GSEA enrichment. FIG. 10C shows an example GSEA comparing a carboplatin-paclitaxel response composite expression pathway signature (reference) and carboplatin-paclitaxel response composite methylation pathway signature (query, p<0.001) based on the analysis in the TCGA-LUAD patient cohort. The horizontal red bar in the top left corner indicates leading edge pathways that are altered on both genomic and epigenomic levels. The NES and p-value were estimated using 1,000 pathway permutations. FIG. 10D shows an example ROC analysis comparing ability of the 7 candidate pathways to predict carboplatin-paclitaxel where their activity is defined based on their expression values (green) or methylation values (blue). The AUROC is indicated

Defining epigenomic signatures of chemotherapy response: The systems and methods were applied to evaluate the response to standard-of-care chemotherapy combination carboplatin and paclitaxel (carboplatin-paclitaxel) in LUAD patients. For this analysis, clinical and molecular profiles of patients with LUAD in the TCGA clinical cohort were analyzed (Cancer Genome Atlas Research Network, Nature. 2014; 511(7511):543-50). To study primary resistance to this chemo combination, patients were selected that did not receive neoadjuvant therapy, were treated with adjuvant carboplatin-paclitaxel chemo regimen, and were further monitored for disease progression (n=14) (Example 20). Each patient that received carboplatin-paclitaxel was evaluated for his/her time to tumor relapse, which was defined as the time between the start of carboplatin-paclitaxel administration and a new tumor event (defined as tumor reappearance or local or distant metastases). To accurately determine a signal that differentiates poor from favorable treatment responses, responder and non-responder analyses were used (such as in Panja et al., EBioMedicine. 2018; 31:110-121), and the tails of the therapeutic response distributions were compared to capture the most prominent molecular signal that differentiates these treatment response groups. To ensure that the comparison groups were balanced with respect to initial age, gender, tumor aggressiveness, smoking status, etc., stratified sub-sampling was performed (which identifies patient groups with similar distributions for these variables), and patients that experienced relapse within 1 year of carboplatin-paclitaxel start (poor response, n=4) as well as patients that did not experience any events for more than 2 years (favorable response, n=4) were identified (Table 1).

To uncover a complex interplay between genomic and epigenomic mechanisms implicated in response to chemotherapy, poor response and favorable response groups were compared based on mRNA expression and DNA methylation profiles using two-sample two-tailed Welch t-test (Welch, Biometrika. 1947; 34(1-2):28-35) (see Example 28), which yielded a carboplatin-paclitaxel response differential gene expression signature and carboplatin-paclitaxel response differential methylation signature. Top differentially expressed genes in the carboplatin-paclitaxel response differential gene expression signature included WWC3, which is a therapeutic target in lung cancer (Han et al., OncoTargets and therapy. 2018; 11:2581-91); CDR1, which is a biomarker in prostate cancer (Salemi et al., The International journal of biological markers. 2014; 29(3):e288-90); FCGBP, which is a potential therapeutic target in metastatic colorectal cancer (Qi et al., Oncology Letters. 2016; 11(1):568-74); and DPYSL2, PTK2 (Bhattacharjee et al., Proceedings of the National Academy of Sciences of the United States of America. 2001; 98(24):13790-5), and DUSP6 (Chen et al., Journal of the National Cancer Institute. 2011; 103(24):1859-70), which are prognostic markers of lung cancer. Genes that harbored top differentially methylated sites in the carboplatin-paclitaxel response differential methylation signature included hypermethylated LAMB3, which is a biomarker of lung cancer (Belinsky S A., Nature reviews Cancer. 2004; 4(9):707-17); CD63, which is a predictive biomarker of LUAD (Kwon et al., Lung cancer. 2007; 57(1):46-53); HES4, which is a prognostic biomarker of osteosarcoma (McManus et al., Pediatric blood & cancer. 2017; 64(5); DAXX, which is a therapeutic target in metastatic lung cancer (Lin et al., Nature Communications. 2016; 7:13867); TSPO, which is a molecular target for tumor imaging and chemotherapy (Austin et al., The international journal of biochemistry & cell biology. 2013; 45(7):1212-6); REG1A, H2AFZ (Beer et al., Nature medicine. 2002; 8(8):816-24), POLG2 (Larsen et al., Clin. Can. Res. 2007; 13(10):2946-54), TOM1L1 (Bhattacharjee et al., Proceedings of the National Academy of Sciences of the United States of America. 2001; 98(24):13790-5) and MB (Zhu et al., J. Clin. Oncol. 2010; 28(29):4417-24), which are known prognostic markers of lung cancer.

Integrative analysis identified epigenomic pathways implicated in resistance: To understand molecular mechanisms that govern chemoresponse, molecular pathways that control genomic and epigenomic signatures of carboplatin-paclitaxel resistance were identified. For this analysis, the carboplatin-paclitaxel response differential expression signature and carboplatin-paclitaxel response differential methylation signature were subjected to a pathway enrichment analysis using the C2 pathway database (which includes the REACTOME (Fabregat et al., Nucleic Acids Res. 2016; 44(D1):D481-7), KEGG (Ogata et al., Nucleic Acids Res. 1999; 27(1):29-34), and BIOCARTA (Nishimura D., Biotech Software & Internet Report: The Computer Software Journal for Scient. 2001; 2(3):117-20) pathways). Pathway enrichment was performed using Gene Set Enrichment Analysis (GSEA) (Subramanian et al., PNAS. 2005(102):15545-50), in which each pathway is assigned a score (i.e., Normalized Enrichment Score, NES) that reflects the level of enrichment in the signature of resistance, also referred to as pathway activity, for the pathway. A list of 833 pathways ranked by their enrichment (NESs) in the carboplatin-paclitaxel response differential expression signature was used to determine the carboplatin-paclitaxel response differential expression pathway signature, and a list of 833 pathways ranked by their enrichment (NESs) for the carboplatin-paclitaxel response methylation signature were used to determine the carboplatin-paclitaxel response differential methylation pathway signature (see Methods). To account for the pathways that have majority of their genes affected in the same direction (such as over-expressed or under-expressed) and pathways that have some genes affected in one direction (such as over-expressed) and some in an opposite direction (such as under-expressed), both signed and absolute valued pathway enrichment analyses were performed with subsequent integration (see Example 28), which were used to determine the carboplatin-paclitaxel response composite expression pathway signature and carboplatin-paclitaxel response composite methylation pathway signature.

Further, to determine interplay between complex mechanisms implicated in chemoresistance, molecular pathways were identified that are affected at both genomic (such as mRNA expression) and epigenomic (such as DNA methylation) levels and that capture pathways with genes affected (i) only at the genomic level, (ii) only at the epigenomic level, (iii) or at both levels (as in FIG. 9). To achieve this goal (FIG. 10A), the carboplatin-paclitaxel response composite expression pathway signature and carboplatin-paclitaxel response composite methylation pathway signature were compared using GSEA, where the carboplatin-paclitaxel response composite expression pathway signature was used as a reference and the carboplatin-paclitaxel response composite methylation pathway signature was used as a query pathways set (the threshold for the query pathway was p-value<0.001 as shown in FIG. 10B, Example 28), which were used to identify 7 molecular pathways with significant alterations at both genomic and epigenomic levels (NES=2.75, p-value<0.001) (FIG. 10C, Example 28). The pathways include (i) chemokine receptors bind chemokines (Fabregat et al., Nucleic Acids Res. 2016; 44(D1):D481-7), (ii) mRNA splicing (Fabregat et al., Nucleic Acids Res. 2016; 44(D1):D481-7), (iii) G alpha signaling events (Fabregat et al., Nucleic Acids Res. 2016; 44(D1):D481-7), (iv) intestinal immune network for IgA production (Ogata et al., Nucleic Acids Res. 1999; 27(1):29-34), (v) metabolism of proteins (Fabregat et al., Nucleic Acids Res. 2016; 44(D1):D481-7), (vi) RNA degradation (Ogata et al., Nucleic Acids Res. 1999; 27(1):29-34), and (vii) cell cycle mitotic (Fabregat et al., Nucleic Acids Res. 2016; 44(D1):D481-7).

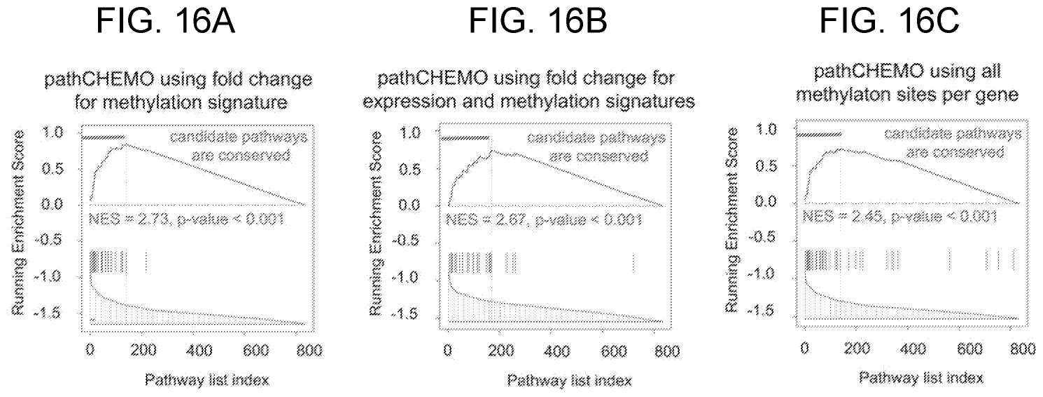

FIG. 16 is related to FIG. 10 and shows that comparative testing of treatment response signatures demonstrates their robustness. GSEAs comparing (a) treatment response composite expression pathway signature (reference) and treatment response composite methylation pathway signature constructed considering all CpG DNA methylation sites (query), (b) treatment response composite expression pathway signature (reference) and treatment response composite methylation pathway signature (query), where methylation signature was defined using fold change, and (c) treatment response composite expression pathway signature (reference) and treatment response composite methylation pathway signature (query), where both signatures were defined using fold change. Horizontal red bars (at the top) indicate leading edge pathways altered on both transcriptomic and epigenomic levels. NES and p-value were estimated using 1,000 pathway permutations.

To confirm that these identified seven molecular pathways are robust to the choice of the statistical methods used to define treatment response signatures, we have also performed our analysis using signatures defined using all DNA methylation sites and using non-parametric tests. First, we defined differential methylation signature with all DNA methylation sites considered (Supplementary FIG. 2a). Second, we defined differential methylation signature using fold change (Supplementary FIG. 2b). Finally, we defined both differential expression and differential methylation signatures using fold change (Supplementary FIG. 2c). Analyses using all of these signatures identified the same seven candidate pathways (GSEA NES>2.45, p-value<0.001), demonstrating robustness of our analysis regardless of the signature choice.

To investigate whether mRNA expression or DNA methylation carries a more significant weight in the predictive ability of the 7 candidate pathways, a ROC analysis was performed based on pathway activities in each patient sample defined on either (i) expression levels or (ii) methylation levels of the pathway genes (Example 28). The analysis demonstrated that both expression levels (AUROC=0.987) and methylation levels (AUROC=0.965) of 7 candidate pathways are highly predictive of poor response vs favorable response separation (FIG. 10D), indicating that they both can be used to identify patients at risk of developing chemoresistance.

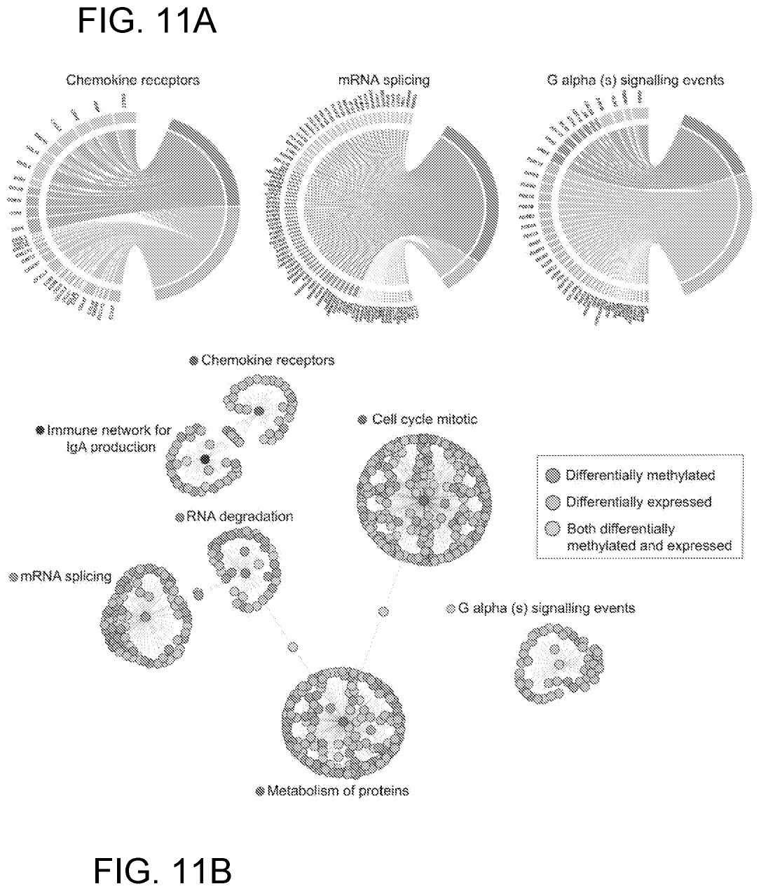

FIGS. 11A-11B show example epigenomic alterations in candidate molecular pathways of carboplatin-paclitaxel response. FIG. 11A shows example molecular pathways altered on both genomic and epigenomic levels, visualized through circlize (Gu et al., Bioinformatics. 2014; 30(19):2811-2) R package. Genes from the leading edge in each pathway are represented as differentially expressed (pink), methylated (grey), and both differentially expressed and methylated (yellow). The width of each connecting line is proportional to the extent of differential expression and differential methylation. From left to right, (left) chemokine receptors bind chemokines pathway (19 differentially expressed genes, 4 differentially methylated genes, and 8 differentially expressed and methylated genes), (middle) mRNA splicing pathway (21 differentially expressed genes, 39 differentially methylated genes, and 28 differentially expressed and methylated genes), and (right) G alpha signaling events pathway (37 differentially expressed genes, 8 differentially methylated genes, and 4 differentially expressed and methylated genes). FIG. 11B shows an example 7-candidate pathway network representation, in which nodes correspond to the genes, which are connected to central pathway-membership circles (i.e., indicating pathway membership). Gene colors describe differential expression (pink), differential methylation (grey), and both differential expression and methylation (yellow). An example network was constructed using igraph (Csardi et al., InterJournal, Complex Systems. 2006; 1695(5):1-9), sna (Butts C T., Social Network Analysis with sna. 2008. 2008; 24(6):51), ggplot2 (Wickham, J Stat Softw. 2010; 35(1):65-88) and ggnetwork (Briatte F. Ggnetwork: Geometries to Plot Networks with ‘ggplot2’2016 (R package version 0.5.1.) R packages.

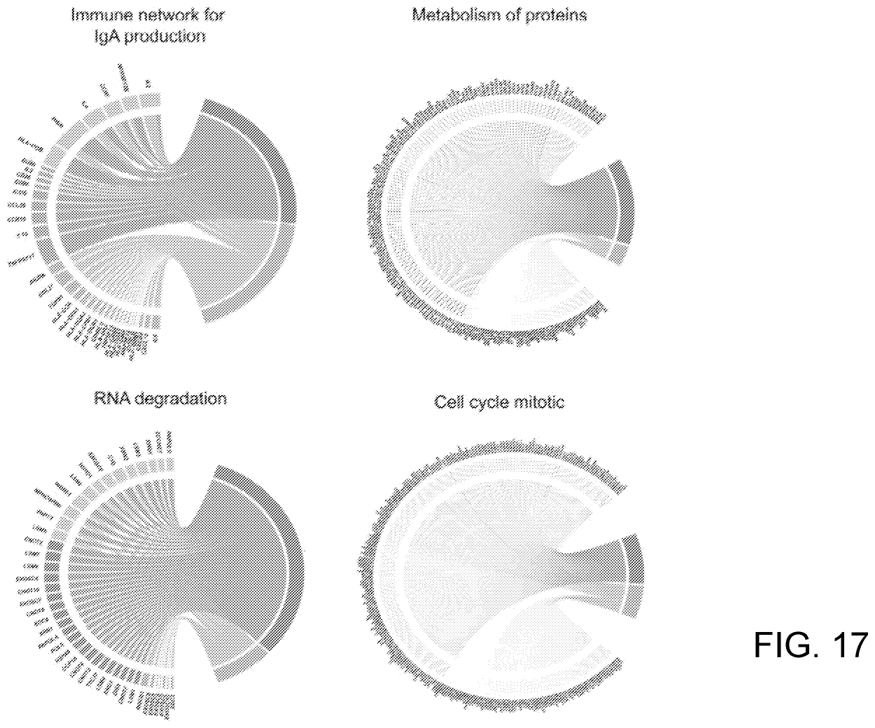

FIG. 17 (related to FIG. 11) shows transcriptomic and epigenomic alterations in selected candidate molecular pathways of carboplatin-paclitaxel resistance. Representative molecular pathways altered on both transcriptomic and epigenomic levels. Genes from the leading edge in each pathway are represented as differentially expressed (pink at bottom right), methylated (grey at top-middle right) and both differentially expressed and methylated (yellow, top left). Width of each connecting line is proportional to the extent of differential expression and differential methylation. Pathways are depicting as follows: (i) intestinal immune network for IgA production pathway (20 differentially expressed genes, 9 differentially methylated genes, and 6 differentially expressed and methylated genes), (ii) metabolism of proteins pathway (47 differentially expressed genes, 53 differentially methylated genes, and 62 differentially expressed and methylated genes), (iii) RNA degradation pathway (7 differentially expressed genes, 21 differentially methylated genes, and 13 differentially expressed and methylated genes), and (iv) cell cycle mitotic pathway (75 differentially expressed genes, 64 differentially methylated genes, and 100 differentially expressed and methylated genes). Pathways were visualized using circlizel package in R.

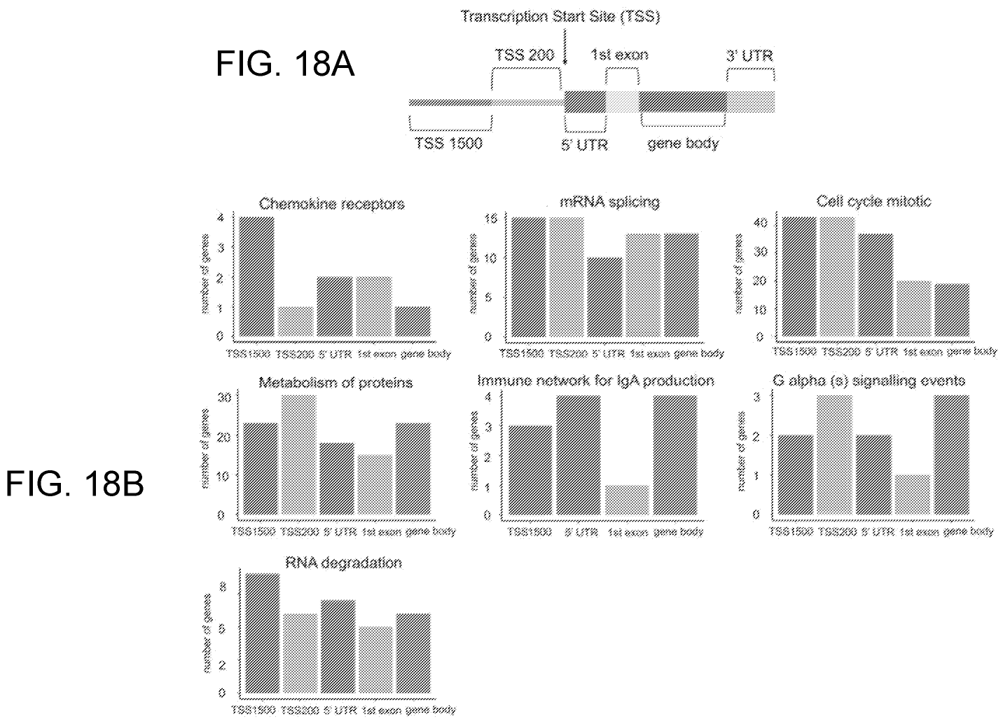

Further evaluated was a topological structure of genomic and epigenomic alterations within each identified pathway. First, the extent to which genes from each pathway were affected on genomic or on epigenomic levels was evaluated (FIG. 11A, FIG. 17), and 7 pathways exercised different patterns of genomic and epigenomic alterations. For example, majority of genes from the G alpha signaling events pathway were altered at the mRNA level (FIG. 11A, nodes in pink at the bottom right of the 3 circle-shaped graphs), while genes from the mRNA splicing pathway were heavily altered at the DNA methylation level (FIG. 11A, nodes in grey) and at both mRNA expression and DNA methylation levels (FIG. 11A, nodes in yellow). Second, connectivity was examined within and between the pathway genes, in which an edge within the pathway corresponds to the pathway membership and a connecting edge between pathways shows shared genes and demonstrates that the candidate pathways share little overlap (FIG. 11B). Finally, differentially methylated sites harbored in genes from the 7 pathways were examined and their regions/locations on the genome were evaluated (FIG. 18A), in which regions were defined as TSS200 (200 base pairs upstream of transcription start site, TSS), TSS1500 (1500 base pairs upstream of TSS200), 5′UTR, 1st exon, gene body, and 3′UTR. In fact, the majority of pathways have methylated sites overrepresented in TSS200+TSS1500 regions, indicating a possible interaction with the transcription machinery binding at the promoter/enhancer regions (Zhang et al., Nucleic Acids Res. 1986; 14(21):8387-97). An exception was the Immune network for IgA production pathway, in which sites were heavily enriched in the gene body, indicating their potential interaction with alternative splicing machinery (Laurent et al., Genome research. 2010; 20(3):320-31) (FIG. 18B).

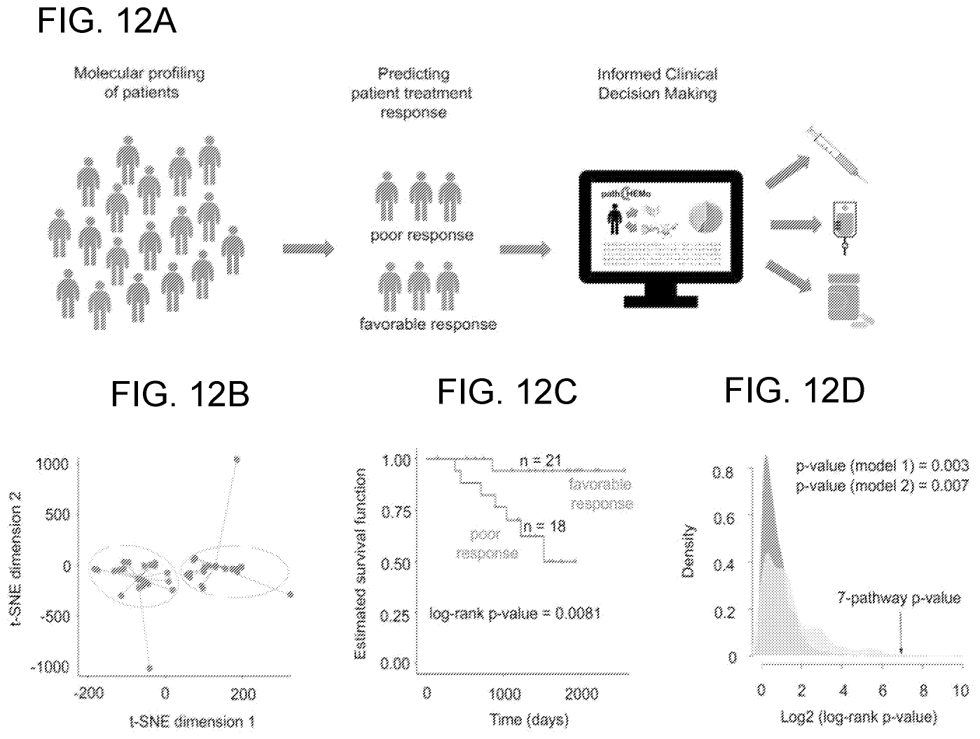

FIGS. 12A-12D show that example candidate molecular pathways stratify patients based on response to carboplatin-taxane in an independent cohort. FIG. 12A shows an example validation strategy. From left to right, (left) molecular epigenomic profiling of patients, (middle) predicting patients' risk of developing chemoresistance, and (right) informed clinical decision making based on patients personalized risks. FIG. 12B shows example t-SNE clustering of lung adenocarcinoma patients treated with carboplatin-taxane (e.g., paclitaxel) from the Tang et al. (Tang et al., Clinical cancer research 2013; 19(6):1577-86) validation cohort (n=39), based on activity levels of 7 candidate pathways. Among the two groups, the green group corresponds to patients with low composite activity levels of candidate pathways, and the orange group corresponds to patients with high composite activity levels of candidate pathways. FIG. 12C shows an example Kaplan-Meier survival analysis used to estimate differences in response to carboplatin-taxane (e.g., paclitaxel) between the two patient groups in identified in (FIG. 12B). A log-rank p-value and the number of patients in each group are indicated. FIG. 12D shows two example random models that indicate the non-random predictive ability of the model in the Tang et al. validation cohort: random model 1 (steel-blue) is defined based on to 7 pathways selected at random, and random model 2 (goldenrod) is defined based on to equally-sized patient groups selected at random.

Validation in independent patient cohorts: The next step was to evaluate if the candidate molecular pathways can stratify patients based on the risk of failing chemotherapy in an independent, non-overlapping patient cohort (FIG. 12A). For this analysis, the Tang et al. cohort (Tang et al., Clin. Can. Res. 2013; 19(6):1577-86) (Example 20) from the University of Texas MD Anderson Cancer Center was considered, which contains LUAD tumor samples obtained at surgery (n=39) collected between 1996 to 2007, followed by treatment with carboplatin and a taxane (e.g., paclitaxel), and monitored for further disease progression for 11 years. In this cohort, survival status during the clinical study (1996 to 2007) was used as a clinical endpoint, and the time to this event was calculated between the start of carboplatin-paclitaxel treatment to death (for patients with this event) or to follow-up (for censored patients). Similar to the analysis above, activity levels of 7 candidate pathways in each patient sample were evaluated (such as through a single-sample pathway analysis, Example 28), and t-SNE clustering was employed, which stratified patients into two groups based on pathway activity levels (FIG. 12B): one group with increased composite pathways' activities (orange) and one group with decreased composite pathways' activities (green). These patient groups were then subjected to a Kaplan-Meier survival analysis (Kaplan et al., Journal of the American Statistical Association. 1958; 53(282):457-81) and a Cox proportional hazards model (Cox D R., Journal of the Royal Statistical Society Series B (Methodological). 1972; 34(2):187-220) (FIG. 12C), which demonstrated a significant difference in the groups' responses to carboplatin-paclitaxel (log-rank p-value=0.0081, hazard ratio=10) (Example 28).

To evaluate the non-randomness of this result, the predictive ability of the 7 candidate pathways was compared with the predictive ability of 7 pathways selected at random (Example 28), which demonstrated that the candidate 7 pathways predict the carboplatin-paclitaxel response non-randomly compared with 10,000 randomly selected pathways (FIG. 12D, random model 1: p-value=0.003). This analysis paralleled and evaluation of whether patient groups stratified by the model showed a significantly different treatment response compared with patient groups chosen at random, which were shown to be non-random (FIG. 12D, random model 2: p-value=0.007).

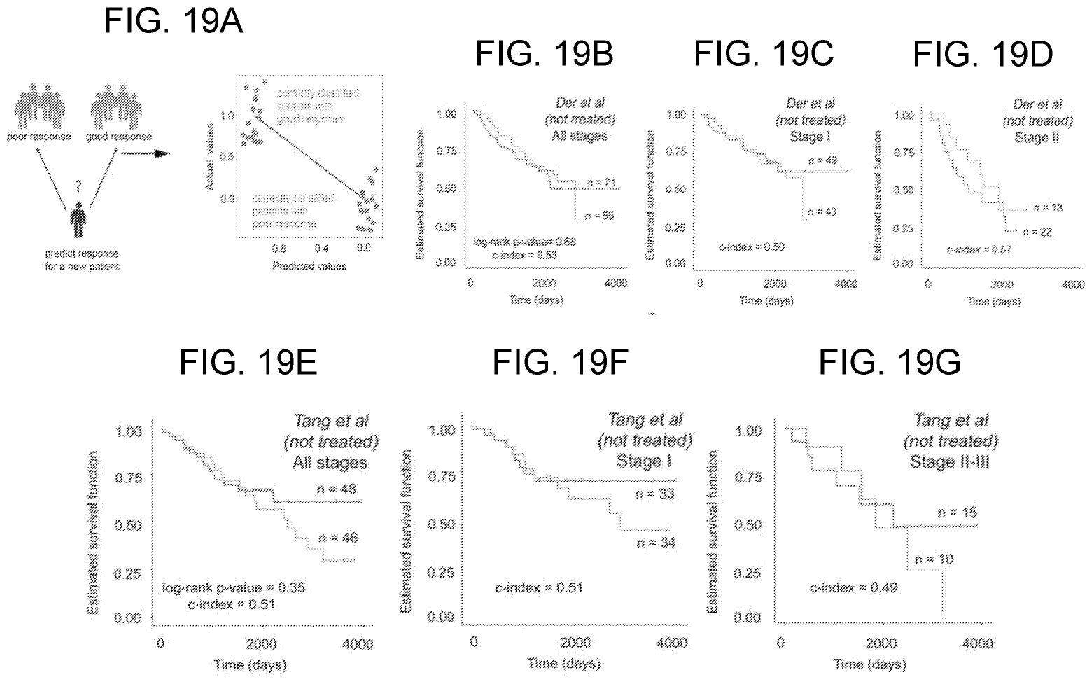

Further, a situation was simulated in which a new incoming patient is diagnosed with LUAD and needs to be assigned risk of developing resistance to carboplatin-paclitaxel utilizing leave-one-out cross-validation (LOOCV) (Stone M., Journal of the royal statistical society Series B (Methodological). 1974:111-47) in the Tang et al. validation cohort. (Tang et al., Clin. Can. Res. 2013; 19(6):1577-86). In LOOCV, one patient is “removed”, and the model is trained on the rest of the patients. The patient that was removed is subjected to predictive analysis and is assigned a risk of developing resistance (simulating a scenario of a new incoming patient). This process was repeated for all patients (Example 28). The LOOCV analysis demonstrated that the systems and methods disclosed herein exhibit high accuracy at predicting poor and favorable carboplatin-paclitaxel responses for new incoming patients (FIG. 19A).

Finally, to show that the candidate pathways distinguish carboplatin-paclitaxel response and not disease aggressiveness, whether the pathways can also separate patients based on their lung cancer aggressiveness was examined. For this analysis, the predictive ability of the candidate pathways was examined for the LUAD patient cohorts that did not receive treatment after surgery (these cohorts were considered negative controls). The datasets (FIG. 15) included (i) Der et al. (Der et al., J. Thor. Oncol. 2014; 9(1):59-64) LUAD tumor samples (n=127) collected through surgery between 1996 to 2005 at Princess Margaret Cancer Centre and (ii) the Tang et al. (Tang et al., Clin. Can. Res. 2013; 19(6):1577-86) provisional cohort, which includes LUAD tumor samples (n=94) collected through surgery between 1996 to 2007 at The University of Texas MD Anderson Cancer Center. These negative control patient cohorts did not receive subsequent treatment but were monitored for disease progression (for Der et al., lung cancer-related death was used as a clinical endpoint, and, for Tang et al., survival status during clinical study (1996 to 2007) was used as a clinical endpoint). A Kaplan-Meier survival analysis on these datasets demonstrated that the candidate 7 pathways did not separate patients based on the disease progression in both unstratified and stratified (based on tumor stages) analyses, (i) Der et al. (FIGS. 19B-19D, log-rank p-value=0.68) and (ii) Tang et al. (FIGS. 19E-19G, log-rank p-value=0.35); thus, the 7 candidate pathways are specific for a carboplatin-paclitaxel response.

Comparison to other methods, signatures of aggressiveness, and common covariates: To assess the advantages of the systems and methods herein, (i) the predictive performance was compared with other commonly utilized approaches, including methods based on linear regression modeling, support vector machine (SVM), and random forest; and (ii) whether the systems and methods disclosed herein can be affected by commonly utilized covariates or known signatures of lung cancer aggressiveness was examined.

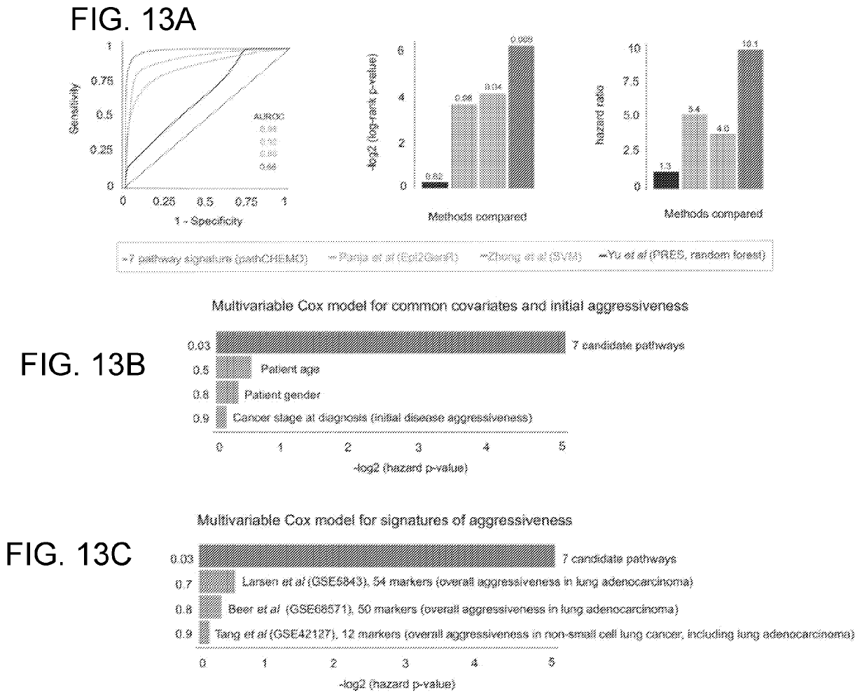

FIGS. 13A-D show an example comparative performance analysis that confirms the significant predictive ability of technologies described herein. FIGS. 13A-13B show a comparison of pathCHEMO (turquoise) to other commonly utilized methods, including Panja et al. (Panja et al., EBioMedicine. 2018) Epi2GenR (yellow), Zhong et al. (Zhong et al., Scientific reports. 2018; 8(1):12675) SVM (light blue), Yu et al. (Yu et al., Scientific reports. 2017; 7:43294) PRES random forest (dark blue) using (FIG. 13A) ROC analysis (with AUROC indicated) and (FIG. 13B) Kaplan-Meier and Cox proportional hazards model (with log-rank p-value and hazard ratio indicated) in Tang et al. validation cohort. FIG. 13C shows an example multivariable Cox proportional hazards analysis demonstrating adjustment of 7 candidate pathways for common covariates (i.e., age, gender and stage at diagnosis). The hazard p-value is indicated. FIG. 13D shows an example multivariable Cox proportional hazards analysis, demonstrating an adjustment of 7 candidate pathways for signatures of lung cancer aggressiveness, including Larsen et al. (Larsen et al., Clin. Can. Res. 2007; 13(10):2946-54) (54 lung adenocarcinoma markers), Beer et al. (Beer et al., Nature medicine. 2002; 8(8):816-24), (50 lung adenocarcinoma markers), and Tang et al. (Tang et al., Clin. Can. Res. 2013; 19(6):1577-86), (12 non-small cell lung cancer markers). The hazard p-value is indicated.

First, to measure the advantage of the systems and methods disclosed herein over other commonly utilized methods, the predictive performance of the systems and methods disclosed herein was compared (Example 28) with (i) Panja et al. (Panja et al., EBioMedicine. 2018; 31:110-121), Epi2GenR based on linear regression integration between DNA methylation and mRNA expression patient profiles, which identified 35 site-gene pairs as candidate markers of carboplatin-paclitaxel response; (ii) Zhong et al. (Zhong et al., Scientific reports. 2018; 8(1):12675), based on a support vector machine (SVM) analysis, which identified 104 candidate genes; and (iii) Yu et al. (Yu et al., Scientific reports. 2017; 7:43294), PRES, based on a random forest algorithm, which identified 3 candidates for the carboplatin-paclitaxel response. The abilities of the identified candidates from each method to separate patients with poor and favorable carboplatin-paclitaxel responses were compared using the Tang et al. dataset and an ROC analysis, which demonstrated the advantage of pathCHEMO over other commonly utilized methods (FIG. 13A, AUROCpathCHEMO=0.98, AUROCEpi2GenR=0.92, AUROCSVM=0.86, AUROCPRES=0.66). Furthermore, the ability of these methods to predict responses to carboplatin-paclitaxel was compared using the Tang et al. validation set, as above, and a Kaplan-Meier survival analysis (FIG. 13B (left), log-rank p-valuepathCHEMO=0.008, log-rank p-valueEpi2GenR=0.04, log-rank p-valueSVM=0.06, log-rank p-valuePRES=0.82) as well as a Cox proportional hazards model (FIG. 13B (right), hazard ratiopathCHEMO=10.1, hazard ratioEpi2GenR=4.0, hazard ratioSVM=5.4, hazard ratioPRES=1.3), which confirmed that, for the Tang et al. validation set, pathCHEMO outperformed other commonly used methods in the ability to predict a therapeutic response.

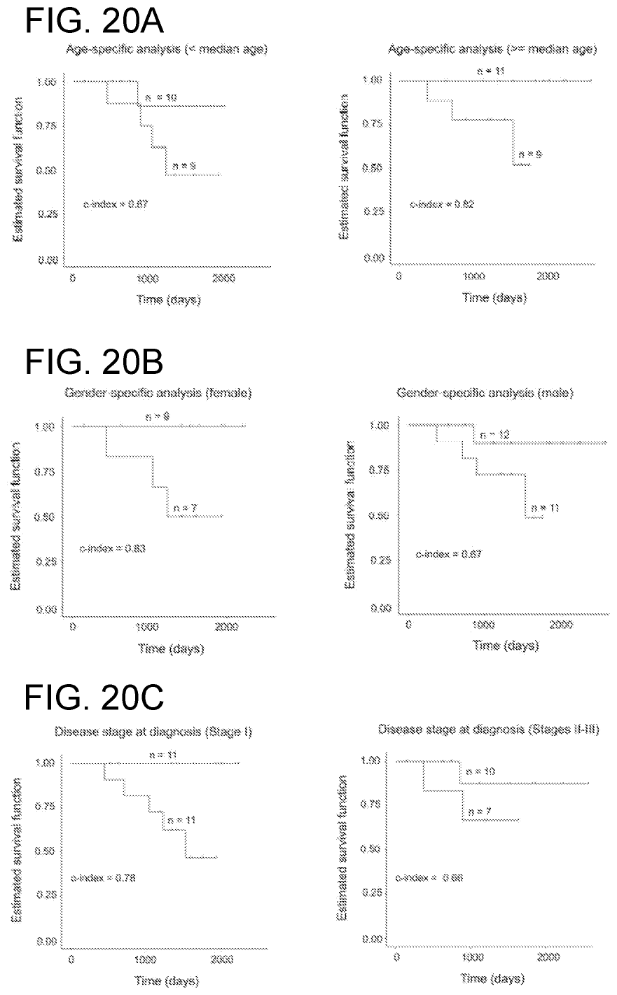

Second, to ensure that the model is not affected by commonly utilized covariates (such as age, gender, and tumor stage at diagnosis), their effect was evaluated through a multivariable (adjusted) Cox proportional hazards analysis (Cox D R., Journal of the Royal Statistical Society Series B (Methodological). 1972; 34(2):187-220) using the Tang et al. dataset, which demonstrated that these covariates are not predictive of treatment response and do not affect predictive ability of the model (FIG. 13C). To confirm this result, a stratified Kaplan-Meier survival analysis was performed, in which the Tang et al. validation cohort was stratified into patient groups based on (i) age (<median age and >=median age), (ii) gender (female and male), and (iii) tumor stage at diagnosis (stage I and stages II and III), which confirmed the ability of the systems and methods disclosed herein to predict a chemotherapy response does not depend on commonly utilized covariates and is indicative of a therapeutic response to carboplatin-paclitaxel (FIGS. 20A-C, related to FIG. 13).

Finally, to ensure that the systems and methods disclosed herein are not affected by markers of overall tumor aggressiveness, whether known prognostic signatures of lung cancer aggressiveness can predict a carboplatin-paclitaxel response or affect the predictive ability of the systems and methods disclosed herein was examined. For this analysis, known prognostic signatures of lung cancer aggressiveness were selected, including (i) Larsen et al. (Larsen et al., Clin. Can. Res. 2007; 13(10):2946-54) (54 prognostic markers), (ii) Beer et al. (Beer et al., Nature medicine. 2002; 8(8):816-24) (50 prognostic markers), and (iii) Tang et al. (Tang et al., Clin. Can. Res. 2013; 19(6):1577-86) (12 prognostic markers) (FIG. 13D), which were utilized in a multivariable Cox proportional hazards analysis, as described above. The analysis demonstrated that these prognostic signatures were not predictive of a carboplatin-paclitaxel response and do not affect the predictive ability of the 7 candidate pathways (FIG. 13D).

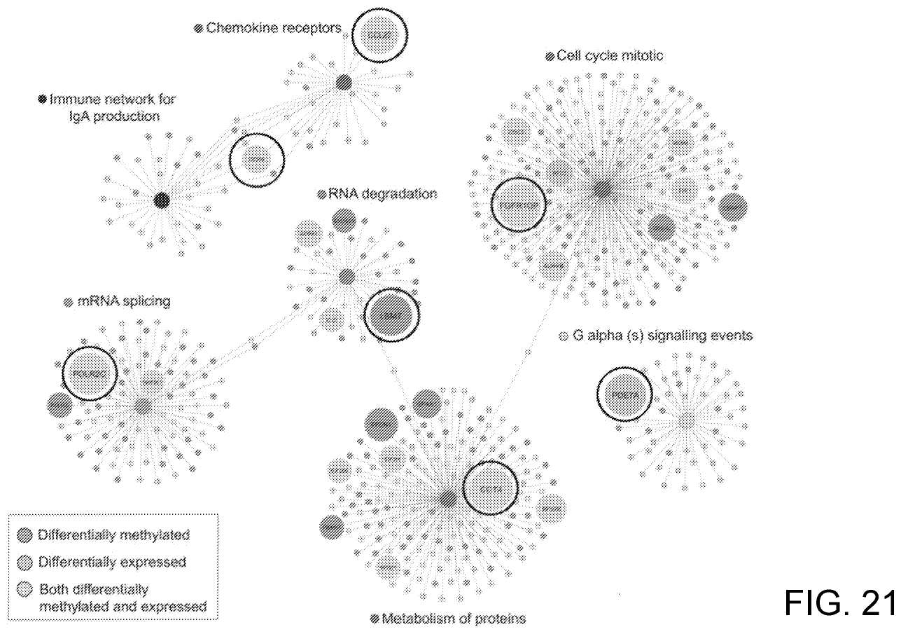

Pathway activity read-outs: Molecular pathways are comprised of multiple genes, which complicate their clinical applicability as markers of treatment response. To tackle this limitation, we looked for genes which could serve as read-outs of pathway's activity implicated in therapeutic response. Specifically, we looked for genes inside each pathway, which were: first, altered on transcriptomic and/or epigenomic levels; second, correlated with pathway activity levels (i.e., NESs in each patient); and finally, associated with carboplatin-paclitaxel response (see Methods). This analysis identified seven read-out genes (i.e., FGFR1OP, CCL22, CCR9, LSM7, PDE7A, CCT4, and POLR2C), which: first, accurately reflected activity levels of their corresponding pathways; second, were associated with treatment response; and finally, achieved identical accuracy in predicting patients at risk of carboplatin-paclitaxel resistance (FIG. 21, related to FIG. 11; Table 6). These seven read-out genes can be used as markers of carboplatin-paclitaxel response and can be easily adopted in the clinic.

Model generalizability: In order to test the general applicability of the systems and methods disclosed herein, they were examined across additional chemotherapy combinations and cancer types. In particular, pathCHEMO was used to determine (i) the cisplatin-vinorelbine response in lung adenocarcinoma; (ii) the cisplatin-vinorelbine response in lung squamous cell carcinoma; and (iii) the folinic acid, fluorouracil, and oxaliplatin (FOLFOX) response in colorectal adenocarcinoma (Example 20, Tables 3, 4, and 5).

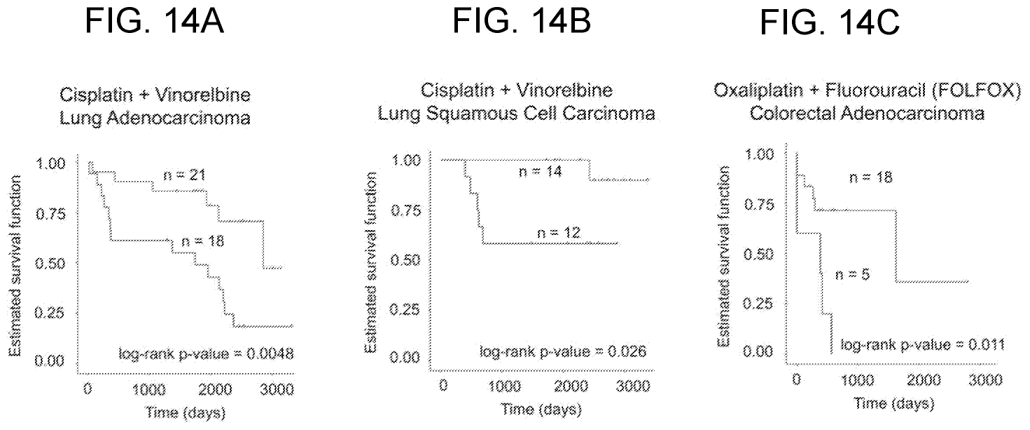

FIGS. 14A-C show that an example implementation (sometimes called “pathCHEMO” herein) accurately identifies pathways of treatment resistance across chemo-regimens and cancer types. Example treatment-related Kaplan-Meier survival analyses are shown as (FIG. 14A) cisplatin-vinorelbine-treated lung adenocarcinoma (LUAD) patients in the Zhu et al. (Zhu et al., J. Clin. Oncol. 2010; 28(29):4417-24) patient cohort (n=39), (FIG. 14B) cisplatin-vinorelbine-treated lung squamous cell carcinoma (LUSC) patients in the Zhu et al. (Zhu et al., J. Clin. Oncol. 2010; 28(29):4417-24) patient cohort (n=26), and (FIG. 14C) FOLFOX (folinic acid, fluorouracil, and oxaliplatin)-treated colorectal adenocarcinoma (COAD) patients in the Marisa et al. patient cohort (n=23), demonstrating the ability of the identified candidate pathways (for each analysis) to predict treatment response. The log rank p-value and number of patients in each group are indicated.

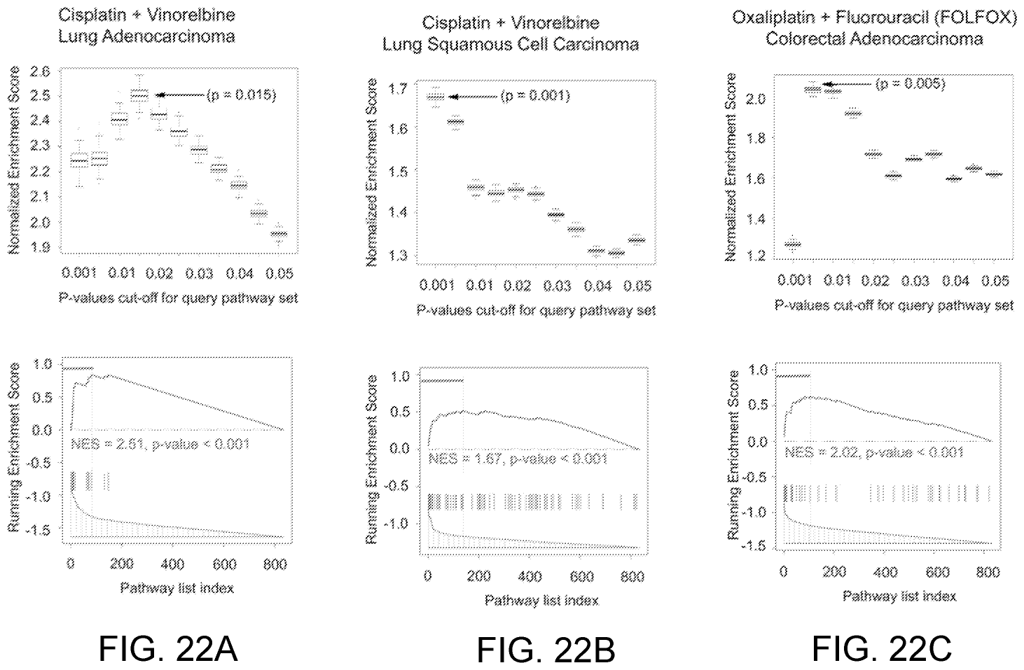

FIGS. 22A-22C show (top row) example box and whisker plots depicting p-value cutoff for a query chemotherapy response composite methylation pathway signature (x-axis) and NESs from the corresponding GSEA comparison between composite methylation and expression pathways signatures (y-axis). The arrow indicates an optimal p-value threshold with the most significant GSEA enrichment. The bottom row shows example GSEAs comparing a chemotherapy response composite expression pathway signature (reference) and methylation pathway signature. The horizontal red bar in the top left corner indicates leading edge pathways that are altered on both genomic and epigenomic levels. The FIG. 22A data are for LUAD patients with cisplatin-vinorelbine chemotherapy, the FIG. 22B data are for LUSC patients with cisplatin-vinorelbine chemotherapy, and the FIG. 22C data are for COAD patients with FOLFOX chemotherapy.

First, the systems and methods disclosed herein were applied to additional chemo combinations (such as cisplatin-vinorelbine), which were administered to lung adenocarcinoma (LUAD) patients, identifying a set of three (3) molecular pathways as markers of cisplatin-vinorelbine resistance (NES=2.51, p-value<0.001) (FIG. 22A) and their corresponding read-out genes (Table 6). These pathways include (i) metabolism of nucleotides (Fabregat et al., Nucleic Acids Res. 2016; 44(D1):D481-7), (ii) actin Y (Nishimura D., Biotech Software & Internet Report: The Computer Software Journal for Scient. 2001; 2(3):117-20), and (iii) ribosome (Ogata et al., Nucleic Acids Res. 1999; 27(1):29-34) pathways, which differ from pathway markers of the carboplatin-paclitaxel response. Next, the predictions were validated using the Zhu et al. (Zhu et al., J. Clin. Oncol. 2010; 28(29):4417-24) patient cohort from the National Cancer Institute of Canada Clinical Trials Group (Example 20), which contains LUAD tumor samples (n=39) collected through surgery for patients that received adjuvant cisplatin-vinorelbine treatment, and the data demonstrate that the three candidate pathways predict poor and favorable cisplatin-vinorelbine responses in patients with LUAD (lung cancer-related death used as a clinical endpoint) using a Kaplan-Meier survival analysis and Cox proportional hazards model (FIG. 14A, log-rank p-value=0.0048, hazard ratio=3.64).

Next, the systems and methods disclosed herein were applied to cisplatin-vinorelbine-treated lung squamous cell carcinoma (LUSC) patients (Table 4), identifying a set of six (6) molecular pathways (NES=1.67, p-value<0.001) (FIG. 22B), including (i) neuroactive ligand-receptor interaction (Ogata et al., Nucleic Acids Res. 1999; 27(1):29-34), (ii) SLC-mediated transmembrane transport (Fabregat et al., Nucleic Acids Res. 2016; 44(D1):D481-7), (iii) transport of mature mRNA derived from an intron-containing transcript (Fabregat et al., Nucleic Acids Res. 2016; 44(D1):D481-7), (iv) cytokine-cytokine receptor interaction (Ogata et al., Nucleic Acids Res. 1999; 27(1):29-34), (v) DNA repair (Fabregat et al., Nucleic Acids Res. 2016; 44(D1):D481-7), and (vi) translation (Fabregat et al., Nucleic Acids Res. 2016; 44(D1):D481-7) pathways. The predictions were validated using the Zhu et al. patient cohort (Zhu et al., J. Clin. Oncol. 2010; 28(29):4417-24) (Example 20), which contains LUSC tumor samples (n=26) collected through surgery, for patients that received adjuvant cisplatin-vinorelbine treatment, demonstrating that six candidate pathways can predict poor and favorable cisplatin-vinorelbine responses in patients with LUSC (lung cancer-related death used as clinical endpoint) using a Kaplan-Meier survival analysis (FIG. 14B, log-rank p-value=0.026, hazard ratio=7.94).

Finally, the systems and methods disclosed herein were applied to patients with colorectal adenocarcinoma (COAD) that received the FOLFOX (folinic acid, fluorouracil, and oxaliplatin) combination (Table 5), identifying five (5) molecular pathways as markers of FOLFOX resistance (NES=2.02, p-value<0.001) (FIG. 22C). The pathways included (i) processing capped intron-containing pre mRNA (Fabregat et al., Nucleic Acids Res. 2016; 44(D1):D481-7), (ii) S phase (Fabregat et al., Nucleic Acids Res. 2016; 44(D1):D481-7), (iii) elongation and processing capped transcripts (Fabregat et al., Nucleic Acids Res. 2016; 44(D1):D481-7), (iv) protein metabolism (Fabregat et al., Nucleic Acids Res. 2016; 44(D1):D481-7), and (v) calcium signaling (Ogata et al., Nucleic Acids Res. 1999; 27(1):29-34) pathways and their corresponding read-out genes (Table 6). The predictions were evaluated using an independent patient cohort, Marisa et al. (Marisa et al., PLoS medicine. 2013; 10(5):e1001453) (Example 20) from the French National Cartes d'Identité des Tumeurs (CIT), which contains COAD tumor samples (n=23) collected through surgery followed by adjuvant treatment with FOLFOX and monitoring for further disease progression (locoregional or distant recurrence), which demonstrated that five candidate pathways can predict poor and favorable FOLFOX responses in patients with COAD using Kaplan-Meier survival analysis (FIG. 14C, log-rank p-value=0.01, hazard ratio=6.21).

These analyses demonstrate general applicability of the systems and methods disclosed herein across various chemotherapy combinations and cancer types, improving the field of personalized therapeutic advice for cancer patients and clinical decision support.

### Example 30—Example Implementation (Clinical and Pathological Features)

Table 2, (related to FIGS. 10, 11, 12, 13, 16, 17, 18, 19, 20, and 21). Clinical and pathological features of lung adenocarcinoma (LUAD) patient cohorts treated with carboplatin-taxane (e.g., paclitaxel), used for signature, validation, and negative controls.

TABLE 2

| | Signature | Validation | Negative controls | |
|---|---|---|---|---|
| Description | TCGA | Tang et al. (treated) | Tang et al. (not treated) | Der et al. |
| Accession # | TCGA-LUAD [2] | GSE42127 [3] | GSE42127 [3] | GSE50081 [4] |
| Patients | 14 | 39 | 94 | 127 |
| Sample | surgery | surgery | surgery | surgery |
| collection | | | | |
| Histological | | | | |
| subtype | | | | |
| mixed | 1 | NA | NA | NA |
| acinar | 1 | | | |
| papillary | | | | |
| mucinous | | | | |
| lepidic | | | | |
| solid | | | | |
| NOS | 12 | | | |
| Anatomic | | | | |
| Site | | | | |
| Left-Upper | 5 | NA | NA | NA |
| Left-Lower | 2 | | | |
| Right-Lower | 1 | | | |
| Right-Middle | 2 | | | |
| Right-Upper | 4 | | | |
| Gender | | | | |
| Female | 9 | 16 | 49 | 62 |
| Male | 5 | 23 | 45 | 65 |
| Tumor Stage | | | | |
| (Pathological) | | | | |
| I | 1 | | | |
| IA | 1 | 1 | 31 | 36 |
| IB | | 21 | 36 | 56 |
| II IIA IIB | 4 | | | |
| IIIA IIIB | 4 | 1 | 5 | 7 |
| IV | 1 | 5 | 11 | 28 |
| NA | 1 | 3 | 4 | |
| | 2 | 8 | 5 | |
| | | | 1 | |
| | | | 1 | |
| Smoking | | | | |
| Status | | | | |
| 1 | 2 | NA | NA | NA |
| 2 | 4 | | | |
| 3 | 3 | | | |
| 4 | 5 | | | |
| 5 | | | | |
| 6 | | | | |

Notes:
NA = Not available, NOS = Not otherwise specified.
Smoking status: 1 = lifelong non-smoker (<100 cigarettes smoked in lifetime), 2 = current smoker (includes daily smokers and non-daily smokers (or occasional smokers), 3 = current reformed smoker for >15 years, 4 = current reformed smoker for ≤15 years, 5 = current reformed smoker, duration not specified, and 6 = smoking history not documented.

[2]—Comprehensive molecular profiling of lung adenocarcinoma. Nature 511, 543-550 (2014).

[3]—Tang, H. et al. A 12-gene set predicts survival benefits from adjuvant chemotherapy in non-small cell lung cancer patients. Clinical cancer research: an official journal of the American Association for Cancer Research 19, 15771586 (2013).

[4]—Love, M. I., Huber, W. & Anders, S. Moderated estimation of fold change and dispersion for RNA-seq data with DESeq2. Genome Biology 15, 550 (2014).

Table 3, related to FIGS. 14 and 22. Clinical and pathological features of lung adenocarcinoma (LUAD) patient cohorts treated with cisplatin-vinorelbine, used for signature and validation.

TABLE 3

| | Signature | Validation |
|---|---|---|
| Description | TCGA | Zhu et al. |
| Accession # | TCGA-LUAD [2] | GSE14814 [5] |
| Patients | 8 | 39 |
| Sample | surgery | surgery |
| collection | | |
| Histological | | |
| subtype | | |
| mixed | 6 | |
| acinar | 1 | 9 |
| papillary | | 5 |
| mucinous | | 1 |
| lepidic | | 1 |
| solid | | 9 |
| NOS | 1 | 14 |
| Anatomic | | |
| Site | | |
| Left-Upper | 2 | NA |
| Left-Lower | | |
| Right-Lower | 2 | |
| Right-Middle | 1 | |
| Right-Upper | 3 | |
| Gender | | |
| Female | 5 | 20 |
| Male | 3 | 19 |
| Tumor Stage | | |
| (Pathological) | | |
| I | | |
| IA | | 8 |
| IB | 1 | 14 |
| II | | |
| IIA | 3 | 11 |
| IIB | 1 | 6 |
| IIIA | 2 | |
| IIIB | | |
| IV | 1 | |
| NA | | |
| Smoking | | |
| Status | | |
| 1 | 1 | NA |
| 2 | | |
| 3 | 4 | |
| 4 | 3 | |
| 5 | | |
| 6 | | |

Notes:
NA = Not available, NOS = Not otherwise specified.
Smoking status: 1 = lifelong non-smoker (<100 cigarettes smoked in lifetime), 2 = current smoker (includes daily smokers and non-daily smokers (or occasional smokers), 3 = current reformed smoker for >15 years, 4 = current reformed smoker for ≤15 years, 5 = current reformed smoker, duration not specified, and 6 = smoking history not documented.

[2]—Comprehensive molecular profiling of lung adenocarcinoma. Nature 511, 543-550 (2014).

[5]—Zhu, C. Q. et al. Prognostic and predictive gene signature for adjuvant chemotherapy in resected non-small-cell lung cancer. Journal of clinical oncology: official journal of the American Society of Clinical Oncology 28, 44174424 (2010).

Table 4, related to FIGS. 14 and 22. Clinical and pathological features of lung squamous cell carcinoma (LUSC) patient cohorts treated with cisplatin-vinorelbine, used for signature and validation.

TABLE 4

| | Signature | Validation |
|---|---|---|
| Description | TCGA | Zhu et al. |
| Accession # | TCGA-LUSC [6] | GSE14814 [5] |
| Patients | 8 | 26 |
| Sample | surgery | surgery |
| collection | | |
| Histological | | |
| subtype | | |
| mixed | 8 | 26 |
| acinar | | |
| papillary | | |
| mucinous | | |
| lepidic | | |
| solid | | |
| NOS | | |
| Anatomic | | |
| Site | | |
| Left-Upper | 2 | NA |
| Left-Lower | | |
| Right-Lower | 4 | |
| Right-Middle | 1 | |
| Right-Upper | 1 | |
| Gender | | |
| Female | 1 | 3 |
| Male | 7 | 23 |
| Tumor Stage | | |
| (Pathological) | | |
| I | | 13 |
| IA | | |
| IB | 2 | |
| II | | 13 |
| IIA | 1 | |
| IIB | 4 | |
| IIIA | 1 | |
| IIIB | | |
| IV | | |
| NA | | |
| Smoking | | |
| Status | | |
| 1 | | NA |
| 2 | | |
| 3 | 2 | |
| 4 | 6 | |
| 5 | | |
| 6 | | |

Notes:
NA = Not available, NOS = Not otherwise specified.
Smoking status: 1 = lifelong non-smoker (<100 cigarettes smoked in lifetime), 2 = current smoker (includes daily smokers and non-daily smokers (or occasional smokers), 3 = current reformed smoker for >15 years, 4 = current reformed smoker for ≤15 years, 5 = current reformed smoker, duration not specified, and 6 = smoking history not documented.

[5]—Zhu, C. Q. et al. Prognostic and predictive gene signature for adjuvant chemotherapy in resected non-small-cell lung cancer. Journal of clinical oncology: official journal of the American Society of Clinical Oncology 28, 44174424 (2010).

[6]—Comprehensive genomic characterization of squamous cell lung cancers. Nature 489, 519-525 (2012).

Table 5 is related to FIGS. 14 and 22. Clinical and pathological features of colorectal adenocarcinoma (COAD) patient cohorts treated with FOLFOX (folinic acid, fluorouracil, oxaliplatin), used for signature and validation.

TABLE 5

| | Signature | Validation |
|---|---|---|
| Description | TCGA | Marisa et al. |
| Accession # | TCGA-COAD [7] | GSE39582 [8] |
| Patients | 8 | 23 |
| Sample | surgery | surgery |
| collection | | |
| Histological | | |
| subtype | | |
| Ascending | 1 | NA |
| Colon | | |
| Cecum | 2 | |
| Descending | 1 | |
| Colon | | |
| Sigmoid | 3 | |
| Colon | | |
| NA | 1 | |
| Gender | | |
| Female | 4 | 8 |
| Male | 4 | 15 |
| Tumor Stage | | |
| (Pathological) | | |
| I | | |
| IA | | |
| IB | | |
| II | | |
| IIA | 1 | 2 |
| IIB | | 1 |
| III | 1 | |
| IIIA | 1 | 3 |
| IIIB | 4 | 3 |
| IIIC | 1 | 3 |
| IV | | 11 |

Notes:
NA = Not available.

[7]—Comprehensive molecular characterization of human colon and rectal cancer. Nature 487, 330-337 (2012).

[8]—a, L. et al. Gene expression classification of colon cancer into molecular subtypes: characterization, validation, and prognostic value. PLoS medicine 10, e1001453 (2013).

### Example 31—Example Implementation (Identified Candidate Pathways)

Table 6 (related to FIG. 21): Identified candidate pathways (carboplatin-paclitaxel treated LUAD, cisplatin-vinorelbine treated LUAD, cisplatin-vinorelbine treated LUSC, and FOLFOX (folinic acid, fluorouracil, oxaliplatin) treated COAD) readout, and contribution to cancer.

TABLE 6

| | | | | |
|---|---|---|---|---|
| Cancer | | | | |
| types & | Candidate | | | |
| treatments | pathways | Readout | Source | Contribution to cancer |

| | | | |
|---|---|---|---|
| LUAD_CP | chemokine | CCL22 | promotes bone metastasis in lung cancer |
| | receptors bind | | |
| | chemokines | | |
| | mRNA splicing | POLR2C | therapeutic target in breast cancer |
| | G alpha (s) | PDE7A | prognostic marker of lung cancer |
| | signalling | | |
| | events | | |
| | intestinal | CCR9 | prognostic marker in non-small cell lung |
| | immune | | cancer, etoposide resistance in prostate |
| | network for IgA | | cancer, cisplatin resistance in breast and |
| | production | | ovarian cancers |
| | metabolism of | CCT4 | therapeutic target in lung cancer |
| | proteins | | |
| | RNA | LSM7 | diagnostic marker of thyroid cancer |
| | degradation | | |
| | cell cycle | FGFR1OP | prognostic biomarker and therapeutic target in |
| | mitotic | | lung cancer |
| LUAD_CV | metabolism of | DTYMK | therapeutic target for LKB1-deficient lung cancer |
| | nucleotides | | |
| | actin Y | ARPC1A | novel marker of pancreatic cancer |
| | ribosome | RPLP2 | prognostic marker gynecologic tumor and in |
| | | | gastric cancer |
| LUSC | cytokine- | CCL11 | biomarker of ovarian cancer |
| | cytokine | | |
| | receptor | | |
| | interaction | | |
| | neuroactive | GABRA1 | DNA methylation markers in colorectal cancer |
| | ligand-receptor | | |
| | interaction | | |
| | DNA repair | ERCC1 | prognostic marker in prostate, and bladder cancer |
| | SLC-mediated | SLC44A4 | novel target for prostate and pancreatic cancer |
| | transmembrane | | |
| | transport | | |
| | translation | RPL14 | molecular marker for esophageal squamous |
| | | | cell carcinoma |
| | transport of | U2AF1 | contributes to cancer progression |
| | mature mRNA | | |
| | derived from | | |
| | an intron- | | |
| | containing | | |
| | transcript | | |
| COAD | elongation and | SF3B3 | therapeutic target for ER-positive breast cancer |
| | processing of | | |
| | capped | | |
| | transcripts | | |
| | processing of | PRPF6 | tumor marker in colon cancer |
| | capped intron | | |
| | containing pre | | |
| | mRNA | | |
| | metabolism of | PFDN1 | promotes EMT and lung cancer progression |
| | protein | | |
| | S phase | CDC25B | prognostic marker in non-small cell lung cancer |
| | calcium | MYLK3 | biomarker in ovarian cancer |
| | signaling | | |

Note:
LUAD_CP = lung adenocarcinoma treated with carboplatin and paclitaxel; LUAD_CV = lung adenocarcinoma treated with cisplatin and vinorelbine; LUSC = lung squamous cell carcinoma treated with cisplatin and vinorelbine; COAD = Colon Adenocarcinoma treated with FOLFOX (folinic acid, fluorouracil, oxaliplatin); Source (fourth column): Genes from the leading edge in each pathway are represented as differentially expressed (medium grey), methylated (dark grey) and both differentially expressed and methylated (light grey).

### Example 32—Example Implementation and Executable Code

The following describes an example method and accompanying executable code to implement the technologies. In practice, genomic activity (gene expression) can be represented in an R data structure on which operations can be performed. Epigenomic activity (e.g., methylation) can similarly be represented.

Step 1: Create signed treatment response transcriptomic signature (compare non-responder and responder patient samples, using gene expression matrix)

TABLE 7
Step 1

treatment_response_transcriptomic_signature <−
sapply(rownames(gene_expression_matrix), function(i){ res<−t.test(gene_expression_matrix[i,
which(colnames(gene_expression_matrix) %in% phenol_nonresponders)],
gene_expression_matrix[i, which(colnames(gene_expression_matrix) %in%
pheno2_responders)]); fc<−
mean(2\hat{ }(gene_expression_matrix[i,phenol_nonresponders]))/mean(2\hat{ }(gene_expression_matrix[i,
pheno2_responders])); if(fc < 1) fc <--1/fc ; return(c(res$stat, res$p.value,fc)) })

Step 2: Run signed pathway enrichment analysis using GSEA. Here, use signed treatment response transcriptomic signature as a reference and collection of genes from each pathway as a query gene set (use C2 pathway collection (http://software.broadinstitute.org/gsea/msigdb), which includes 833 pathways from REACTOME, KEGG, and BIOCARTA databases).

TABLE 8
Step 2

pathway_names<−names(C2)[pathways] #833 pathways for(i in
1:length(pathway_names)){ pi<−which(names(C2)==pathway_names[i]) r<−
gsea(treatment_response_transcriptomic_signature[1,], C2[[pi]])
write(c(as.character(names(C2)[pi]), “signed”, r$p.value,r$NES),
file=paste(“pathway_transcriptomic_signature_signed.txt”, sep=“”), sep=‘\t’, ncol=7,append=T) }

Step 3: Create absolute valued treatment response transcriptomic signature

TABLE 9
Step 3

treatment_response_transcriptomic_signature_abs <− abs(treatment_response_tra
nscriptomic_signature)

Step 4: Run absolute valued pathway enrichment analysis using GSEA function. Here, use absolute valued treatment response transcriptomic signature as a reference and collection of genes from each pathway as a query gene set (as above).

TABLE 10
Step 4

for(i in 1:length(pathway_names)){ pi<−which(names(C2)==pathway_names[i]) r<−
gsea(treatment_response_transcriptomic_signature_abs[1,], C2[[pi]])
write(c(as.character(names(C2)[pi]), “signed”, r$p.value,r$NES),
file=paste(“pathway_transcriptomic_signature_abs.txt”, sep=“”), sep=‘\t’, ncol=7,append=T) }

Step 5: Combine signed and absolute valued pathway enrichment analysis (from step 2 and step 4) so that enrichment for each pathway is chosen based on whichever is more significant: enrichment from step 2 or from step 4. Please note, one need not consider negative NES from absolute valued pathway enrichment analysis—as they indicate the absence of enrichment. This defines composite expression pathway signature.

TABLE 11
Step 5

```
  composite_expression_pathway_signature_matrix <− merge(pathway_transcriptomi
c_signature_signed, pathway_transcriptomic_signature_abs, by=‘row.names’, all=T RUE)
rownames(composite_expression_pathway_signature_matrix) <− composite_expressi
on_pathway_signature_matrix[,1] composite_expression_pathway_signature_matrix$Row.names
<− NULL final_matrix <− composite_expression_pathway_signature_matrix(0,833,2) for(i in
1:833){ if((composite_expression_pathway_signature_matrix[i,1] < composite_expressi
on_pathway_signature_matrix[i,3]) & (composite_expression_pathway_signature_m atrix[i,4] <
0)) { final_matrix[i,1] = composite_expression_pathway_signature_matrix[i,1]
  final_matrix[i,2] = composite_expression_pathway_signature_matrix[i,2] } else {
final_matrix[i,1] = composite_expression_pathway_signature_matrix[i,3] final_matrix[i,2] =
composite_expression_pathway_signature_matrix[i,4] } } row.names(final_matrix) <−
rownames(composite_expression_pathway_signature_ma trix) colnames(final_matrix) <−
c(“p.value”, “NES”) composite_expression_pathway_signature <− final_matrix
```

Step 6: For epigenomic analysis (similarly to step 1), create signed treatment response epigenomic signature (compare non-responder and responder patient samples, using DNA methylation matrix).

TABLE 12
Step 6

treatment_response_epigenomic_signature <− sapply(rownames(DNA_methylation_ma
trix), function(i){ res<−t.test(DNA_methylation_matrix[i,
which(colnames(DNA_methylation_matrix ) %in% phenol_nonresponders)],
DNA_methylation_matrix[i, which(colnames(DNA_methylation_matrix ) %in%
pheno2_responders)]); fc<−
mean(2\hat{ }(DNA_methylation_matrix[i,phenol_nonresponders]))/mean(2\hat{ }(DNA_me
thylation_matrix[i,pheno2_responders])); if(fc < 1) fc <--1/fc ; return(c(res$stat, res$p.value,fc))
})

Step 7: Run signed pathway enrichment analysis (similar to step 2), using GSEA. Here, use signed treatment response epigenomic signature as a reference and collection of genes from each pathway as a query gene set.

TABLE 13
Step 7

pathway_names<−names(C2)[pathways]#833 for(i in 1:length(pathway_names)){ pi<−
which(names(C2)==pathway_names[i]) r<−gsea(treatment_response_epigenomic_signature[1,],
C2[[pi]]) write(c(as.character(names(C2)[pi]), “signed”, r$p.value,r$NES),
file=paste(“pathway_epigenomic_signature_signed.txt”, sep=“”), sep=‘\t’, ncol=7,append=T) }

Step 8: Create absolute valued treatment response epigenomic signature (similarly to step 3)

TABLE 14
Step 8

treatment_response_epigenomic_signature_abs <− abs(treatment_response_epigeno
mic_signature)

Step 9: Run absolute valued pathway enrichment analysis, using GSEA function (similarly to step 4). Here, use absolute valued treatment response signature as a reference and collection of genes from each pathway as a query gene set.

TABLE 15
Step 9

for(i in 1:length(pathway_names)){ pi<−which(names(C2)==pathway_names[i]) r<−
gsea(treatment_response_epigenomic_signature_abs[1,], C2[[pi]])
write(c(as.character(names(C2)[pi]), “signed”, r$p.value,r$NES),
file=paste(“pathway_epigenomic_signature_abs.txt”, sep=“”), sep=‘\t’, ncol=7,append=T) }

Step 10: Similarly to step 5, combine signed and absolute valued pathway enrichment analysis from steps 7 and 9 and choose pathway enrichment based on most significant enrichment in either step 7 or 9 (similarly to step 5). This defined composite methylation pathway signature.

TABLE 16
Step 10

composite_methylation_pathway_signature_matrix <− merge(pathway_epigenomic_s
ignature_signed, pathway_epigenomic_signature_abs, by=‘row.names’, all= TRUE)
rownames(composite_methylation_pathway_signature_matrix) <− composite_methyla
tion_pathway_signature_matrix[,1]
composite_methylation_pathway_signature_matrix$Row.names <− NULL final_matrix <−
composite_methylation_pathway_signature_matrix(0,833,2) for(i in 1:833){
if((composite_methylation_pathway_signature_matrix[i,1] < composite_methyla
tion_pathway_signature_matrix[i,3]) & (composite_methylation_pathway_signatur e_matrix[i,4] <
0)) { final_matrix[i,1] = composite_methylation_pathway_signature_matrix[i,1] final_matrix[i,2] =
composite_methylation_pathway_signature_matrix[i,2] } else { final_matrix[i,1] =
composite_methylation_pathway_signature_matrix[i,3] final_matrix[i,2] =
composite_methylation_pathway_signature_matrix[i,4] } } row.names(final_matrix) <−
rownames(composite_methylation_pathway_signature_m atrix) colnames(final_matrix) <−
c(“p.value”, “NES”) composite_methylation_pathway_signature <− final_matrix

Step 11: Finally, to identify pathways that are significantly affected on both transcriptomic and epigenomic levels, one can compare these pathway signatures using GSEA (e.g., pathway-based GSEA). One can use composite expression pathway signature as a reference signature and composite methylation pathway signature as a query set in GSEA. This analysis will give one a list of leading-edge pathways, which are affected on both transcriptomic and epigenomic levels.

TABLE 17
Step 11

```
  path_LUAD_DNA_less0.001 <− row.names(composite_methylation_pathway_signature
[composite_methylation_pathway_signature [,1]<0.001,])
  GSEA_pathCHEMO <−gsea(composite_expression_pathway_signature[,2],
path_LUAD_DNA_less0.001, main.string = “expression (reference) and methylation (query)”,
phenol = “non-responder”, pheno2 = “responder”, plotFile=paste(“GSEA_plot.pdf”, sep=“”))
```

### Example 33—Example Detailed Implementation in R Programming Language: Introduction

The following describes an example detailed implementation of the technologies in the R programming language called “pathCHEMO.” The implementation includes R objects and code. However, the technologies can be practiced in any of a variety of programming languages in practice.

### Example 34—Example Detailed Implementation in R Programming Language: Transcriptomic and Epigenomic Signatures (Treatment Response Signatures)

To elucidate transcriptomic (i.e., differentially expressed) and epigenomic (i.e., differentially methylated) treatment response (i.e., to carboplatin-paclitaxel) in lung adenocarcinoma, we utilized RNA-seq (for mRNA expression) and DNA methylation data from The Cancer Genome Atlas lung adenocarcinoma (TCGA-LUAD) project (Comprehensive molecular profiling of lung adenocarcinoma. Nature 511, 543-550 (2014)) downloaded from Genomics Data Commons database (GDC; https://portal.gdc.cancer.gov/).

To define treatment response signatures, we compared mRNA expression or DNA methylation data between poor and favorable response patient groups using two-sample two-tailed Welch t-test (which can be done in either R using t.test function or for example in excel). For convenience, we are providing complete signature objects (for signed classic signatures) here: treatment response transcriptomic signature is contained in R object treatment response transcriptomic signature and the treatment response epigenomic signature is contained in R object treatment response epigenomic signature.

An example of top 10 differentially expressed genes for the transcriptomic signature:

>treatment_response_transcriptomic_signature[1:10,]

t.stat p.value foldchange

DUOX2 10.47 1e-04 11.65

PHF21A 9.15 1e-04 8.50

E2F1 9.83 1e-04 10.26

FCGBP 8.37 2e-04 6.68

SETDS 6.85 5e-04 5.92

EIF4A3 −7.17 6e-04-8.98

ZNF296 6.74 7e-04 4.69

BTNL8 6.18 8e-04 4.66

FGFR1OP −6.70 8e-04-7.46

RLIM 6.16 9e-04 4.04

And for the epigenomic signature:

>treatment_response_epigenomic_signature[1:10,]

t.stat p.value foldchange

DERL1 13.35 2.06e-05 14.59

NANP 11.13 2.00e-04 14.00

GOSR1 9.32 2.00e-04 10.55

RNASEH2B 9.19 2.00e-04 10.43

FAM76B 8.49 2.00e-04 10.33

GIPR 7.02 4.00e-04 8.67

NR2F2 7.00 4.00e-04 8.57

TSPAN3 6.95 4.00e-04 8.51

RPL26L1 6.91 5.00e-04 8.30

LAMB3 6.90 5.00e-04 8.27

Also, code to define absolute valued signatures and their R objects are as follows:

> #code to define absolute valued treatment response transcriptomic
signature
> treatment_response_transcriptomic_signature_abs <− abs(
+ treatment_response_transcriptomic_signature)
> treatment_response_transcriptomic_signature_abs[1:10,]

t.stat p.value foldchange

DUOX2 10.47 1e-04 11.65

PHF21A 9.15 1e-04 8.50

E2F1 9.83 1e-04 10.26

FCGBP 8.37 2e-04 6.68

SETDS 6.85 5e-04 5.92

EIF4A3 7.17 6e-04 8.98

ZNF296 6.74 7e-04 4.69

BTNL8 6.18 8e-04 4.66

FGFR1OP 6.70 8e-04 7.46

RLIM 6.16 9e-04 4.04

> #code to define absolute valued treatment response epigenomic signature
> treatment_response_epigenomic_signature_abs <− abs(
+ treatment_response_epigenomic_signature)
> treatment_response_epigenomic_signature_abs[1:10,]

t.stat p.value foldchange

DERL1 13.35 2.06e-05 14.59

NANP 11.13 2.00e-04 14.00

GOSR1 9.32 2.00e-04 10.55

RNASEH2B 9.19 2.00e-04 10.43

FAM76B 8.49 2.00e-04 10.33

GIPR 7.02 4.00e-04 8.67

NR2F2 7.00 4.00e-04 8.57

TSPAN3 6.95 4.00e-04 8.51

RPL26L1 6.91 5.00e-04 8.30

LAMB3 6.90 5.00e-04 8.27

### Example 35—Example Detailed Implementation in R Programming Language: Transcriptomic and Epigenomic Signatures

### Transcriptomic and Epigenomic Pathway Signatures

To define molecular pathway signatures altered on transcriptomic and epigenomic levels, we performed pathway enrichment analysis on treatment response transcriptomic signature and treatment response epigenomic signature. We utilized the comprehensive C2 pathway collection http://software.broadinstitute.org/gsea/msigdb (Liberzon, A. et al. Molecular signatures database (MSigDB) 3.0. Bioinformatics (Oxford, England) 27, 1739-1740 (2011)), which includes 833 pathways from REACTOME (Fabregat, A. et al. The Reactome pathway Knowledgebase. Nucleic Acids Res. 44, D481-487 (2016)), KEGG (Ogata, H. et al. KEGG: Kyoto Encyclopedia of Genes and Genomes. Nucleic Acids Res. 27, 29-34 (1999)), and BIOCARTA (Liberzon, A. et al. Molecular signatures database (MSigDB) 3.0. Bioinformatics (Oxford, England) 27, 1739-1740 (2011)) databases. Pathway enrichment analysis can be performed using commonly used tools such as Gene Set Enrichment Analysis (GSEA) (Nishimura, D. Biocarta. Biotech Software and Internet report: The computer software. Journal of Scientist database J Scient. 2001; 2:117-120), which can be downloaded or freely used as an online interactive tool from the Broad Institute http://software.broadinstitute.org/gsea/or other sources. Treatment response signatures (i.e., transcriptomic and epigenomic) should be used as a reference and collection of genes from each pathway should be used as a query gene set (pathways and pathways genes are includes as an automated feature in Broad GSEA, as referenced above).

This analysis estimates normalized enrichment score (NES) for each pathway, depicting how much each pathway is enriched in the treatment response signature and defines a so-called \pathway activity″.

To capture pathways (i) whose genes are strictly over-expressed or under-expressed; and (ii) whose genes are significantly changed in both directions (i.e., such pathway would contain genes that are significantly over-expressed and genes that are significantly under-expressed), we have utilized both signed and absolute valued signatures for pathway enrichment analysis, respectively.

### Example 36—Example Detailed Implementation in R Programming Language: Transcriptomic and Epigenomic Signatures

### Transcriptomic and Epigenomic Pathway Signatures: Signed Pathway Enrichment Analysis

For signed pathway analysis, signed treatment response signature (i.e., transcriptomic or epigenomic) is used as input (i.e., reference signature) for GSEA (as above). A pseudocode below provides overview of what will happen during Broad GSEA execution (or this code can be used directly if GSEA is downloaded locally), when transcriptomic signature signed is used as a reference.

> #code for signed pathway enrichment analysis
> pathway_signature <− NULL
> for(i in 1:length(pathways)){ #833 pathways from C2 database
+ query <− pathway_genes(pathways[i])
+ r <− gsea(transcriptomic_signature_signed, query) # run GSEA
+ pathway_signature [i]<− cbind(r.pvalue, r.NES) #signed pathway
signature
+ }

For convenience, we have included a signed expression pathway signature as pathway_transcriptomic_signature_signed R object:

>pathway_transcriptomic_signature_signed[1:5,]

p.value NES

REACTOME_MITOTIC_M_M_G1_PHASES 0.001 8.793

REACTOME_DOWNSTREAM_EVENTS_IN_GPCR_SIGNALING 0.001 8.718

REACTOME_GPCR_LIGAND_BINDING 0.001 8.233

KEGG_CYTOKINE_CYTOKINE_RECEPTOR_INTERACTION 0.001 7.454

REACTOME_HEMOSTASIS 0.001 7.265

And a signed methylation pathway signature as pathway_epigenomic_signature_signed R object:

>pathway_epigenomic_signature_signed[1:5,]

p.value

REACTOME_ACTIVATION_OF_ATR_IN_RESPONSE_TO_REPLICATION_STRESS 0.001

REACTOME_MRNA_SPLICING_MINOR_PATHWAY 0.001

REACTOME_FORMATION_AND_MATURATION_OF_MRNA_TRANSCRIPT 0.001

REACTOME_MRNA_SPLICING 0.001

REACTOME_FORMATION_OF_THE_TERNARY_COMPLEX_AND_SUBSEQUENTLY_THE_43S_COMPLEX 0.001

NES

REACTOME_ACTIVATION_OF_ATR_IN_RESPONSE_TO_REPLICATION_STRESS 1.927

REACTOME_MRNA_SPLICING_MINOR_PATHWAY 1.894

REACTOME_FORMATION_AND_MATURATION_OF_MRNA_TRANSCRIPT 1.859

REACTOME_MRNA_SPLICING 1.828

REACTOME_FORMATION_OF_THE_TERNARY_COMPLEX_AND_SUBSEQUENTLY_THE_43S_COMPLEX 1.804

### Example 37—Example Detailed Implementation in R Programming Language: Transcriptomic and Epigenomic Signatures

### Transcriptomic and Epigenomic Pathway Signatures: Absolute Valued Pathway Enrichment Analysis

For absolute valued pathway analysis, absolute valued treatment response signature (i.e., transcriptomic or epigenomic) is used as input (i.e., reference signature) for GSEA. As an example, we are providing a pseudocode of what will happen inside Broad GSEA execution (or this code can be used directly if GSEA is downloaded locally), when transcriptomic signature abs is used as a reference.

> #code for absolute pathway enrichment analysis
> pathway_signature <− NULL
> for(i in 1:length(pathways)){ #833 pathways from C2 database
+ query <− pathway_genes(pathways[i])
+ r <− gsea(transcriptomic_signature_abs, query) # run GSEA
+ pathway_signature [i]<− cbind(r.NES, r.pvalue) #absolute pathway
signature
+ }

For convenience, we have included an absolute valued expression pathway signature as pathway_transcriptomic_signature_abs R object:

>pathway_transcriptomic_signature_abs[1:5,

p.value NES

REACTOME_CELL_CYCLE_MITOTIC 0.001 10.128

REACTOME_CELL_CYCLE_CHECKPOINTS 0.001 7.014

REACTOME_MRNA_SPLICING 0.001 4.898

KEGG_PHOSPHATIDYLINOSITOL_SIGNALING_SYSTEM 0.001 4.278

KEGG_RNA_DEGRADATION 0.001 3.864

And a absolute methylation pathway signature as pathway epigenomic signature abs R object:

>pathway_epigenomic_signature_abs[1:5,]

p.value NES

REACTOME_CHEMOKINE_RECEPTORS_BIND_CHEMOKINES 0.001 5.368

KEGG_INTESTINAL_IMMUNE_NETWORK_FOR_IGA_PRODUCTION 0.001 2.941

REACTOME_G_ALPHAS_SIGNALLING_EVENTS 0.001 2.715

REACTOME_CDC6_ASSOCIATION_WITH_THE_ORC:ORIGIN_COMPLEX 0.001 2.144

REACTOME_NUCLEOTIDE_EXCISION_REPAIR 0.001 1.878

### Example 38—Example Detailed Implementation in R Programming Language: Transcriptomic and Epigenomic Pathway Integration

### Composite Pathway Signatures

Next, signed and absolute valued pathway signatures are integrated to define composite expression pathway signature and composite methylation pathway signature, in the following way. For composite expression pathway signature:

```
  > #code for composite expression pathway signature
  > composite_expression_pathway_signature_matrix <−
  + merge(pathway_transcriptomic_signature_signed,
  + pathway_transcriptomic_signature_abs, by=‘row.names’, all=TRUE)
  > rownames(composite_expression_pathway_signature_matrix) <−
  + composite_expression_pathway_signature_matrix[,1]
  > composite_expression_pathway_signature_matrix$Row.names <− NULL
  > final_matrix <− matrix(0,833,2)
  > for(i in 1:833){
  + if(composite_expression_pathway_signature_matrix[i,1] <
  + composite_expression_pathway_signature_matrix[i,3])
  + {
  + final_matrix[i,1] = composite_expression_pathway_signature_matrix[i,1]
  + final_matrix[i,2] = composite_expression_pathway_signature_matrix[i,2]
  + } else { if (composite_expression_pathway_signature_matrix[i,4] < 0)
  + {
  + final_matrix[i,2] = composite_expression_pathway_signature_matrix[i,2]
  + final_matrix[i,1] = composite_expression_pathway_signature_matrix[i,1]
  + } else {
  + final_matrix[i,2] = composite_expression_pathway_signature_matrix[i,4]
  + final_matrix[i,1] = composite_expression_pathway_signature_matrix[i,3]
  + }
  + }
  + }
  > row.names(final_matrix) <− rownames(composite_expression_pathway_signature_matrix)
  > colnames(final_matrix) <− c(“p.value”, “NES”)
  And for composite methylation pathway signature:
  > #code for composite methylation pathway signature
  > composite_methylation_pathway_signature_matrix <−
  + merge(pathway_epigenomic_signature_signed,
  + pathway_epigenomic_signature_abs, by=‘row.names’, all=TRUE)
  > rownames(composite_methylation_pathway_signature_matrix) <−
  + composite_methylation_pathway_signature_matrix[,1]
  > composite_methylation_pathway_signature_matrix$Row.names <− NULL
  > final_matrix <− matrix(0,833,2)
  > for(i in 1:833){
  + if(composite_methylation_pathway_signature_matrix[i,1] <
  + composite_methylation_pathway_signature_matrix[i,3])
  + {
  + final_matrix [i,1] = composite_methylation_pathway_signature_matrix[i, 1]
  + final_matrix[i,2] = composite_methylation_pathway_signature_matrix[i,2]
  + } else { if (composite_methylation_pathway_signature_matrix[i,4] < 0)
  + {
  + final_matrix[i,2] = composite_methylation_pathway_signature_matrix[i,2]
  + final_matrix[i, 1] = composite_methylation_pathway_signature_matrix[i, 1]
  + } else {
  + final_matrix[i,2] = composite_methylation_pathway_signature_matrix[i,4]
  + final_matrix[i,1] = composite_methylation_pathway_signature_matrix[i,3]
  + }
  + }
  + }
  > row.names(final_matrix) <−
rownames(composite_methylation_pathway_signature_matrix)
  > colnames(final_matrix) <− c(“p.value”, “NES”)
```

For convenience, we provide composite expression pathway signature as R object composite_express-

ion_pathway_signature and the composite methylation pathway signature as R object composite_methylation_pathway_signature. Signature description and the size for composite_expression_pathway_sign

ature (similarly to composite_methylation_pathway_signature) can be obtained with:

>colnames(composite_expression_pathway_signature)

[1] “p.value” “NES”

>dim(composite_expression_pathway_signature)

[1] 833 2

In these signatures, row names corresponds to 833 pathway names, _rst column corresponds to p-value

of the pathways, and second column corresponds to normalized enrichment score of the pathways (NES).

>composite_expression_pathway_signature[1:5,]

p.value NES

REACTOME_CELL_CYCLE_MITOTIC 0.001 10.127686

REACTOME_MITOTIC_M_M_G1_PHASES 0.001 8.793161

REACTOME_DOWNSTREAM_EVENTS_IN_GPCR_SIGNALING 0.001 8.717609

REACTOME_GPCR_LIGAND_BINDING 0.001 8.233202 KEGG_CYTOKINE_CYTOKINE_RECEPTOR_INTERACTION 0.001 7.453886

### Example 39—Example Detailed Implementation in R Programming Language: Transcriptomic and Epigenomic Pathway Integration

### Integrative Pathway Analysis

To identify pathways that are significantly affected on both transcriptomic and epigenomic levels, GSEA needs to be utilized (as above), where we used composite expression pathway signature as a reference signature and composite methylation pathway signature as a query set (at p-value cut-o_0.001, estimated as described below).

### Example 40—Example Detailed Implementation in R Programming Language: Transcriptomic and Epigenomic Pathway Integration

### Integrative Pathway Analysis: Define Query Pathway Set

To accurately define query pathway set, we varied the threshold for the composite methylation pathway signature between 0.001 and 0.05 (width of each step=0.005) and estimated the strength of enrichment between the two signatures at each threshold for 100 times, with average NES for each enrichment reported and provided as NES_100_times_for_query R object.

>boxplot(NES_100_times_for_query, xlab=“P-values cut-off for query pathway set”, +ylab=“Normalized Enrichment Score”)

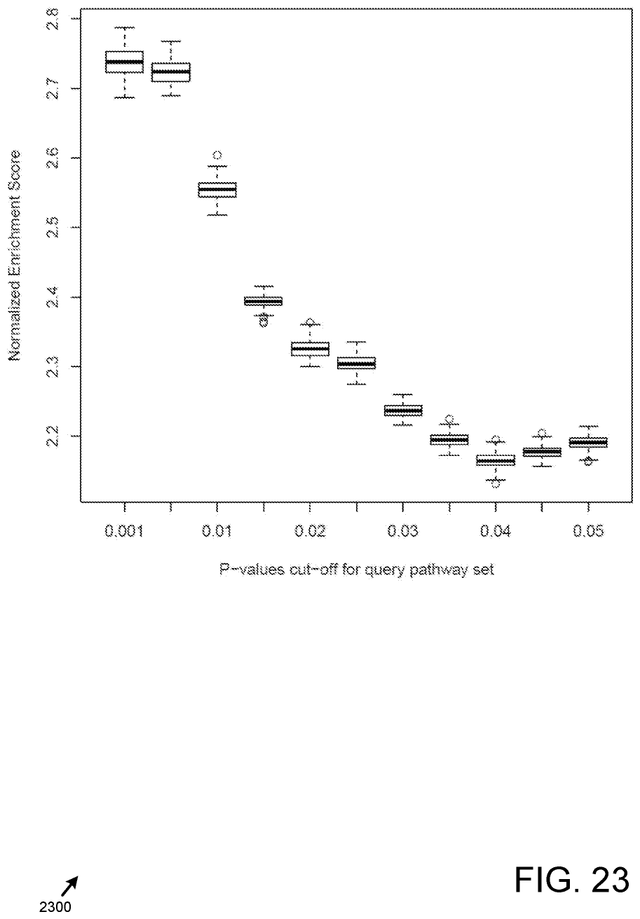

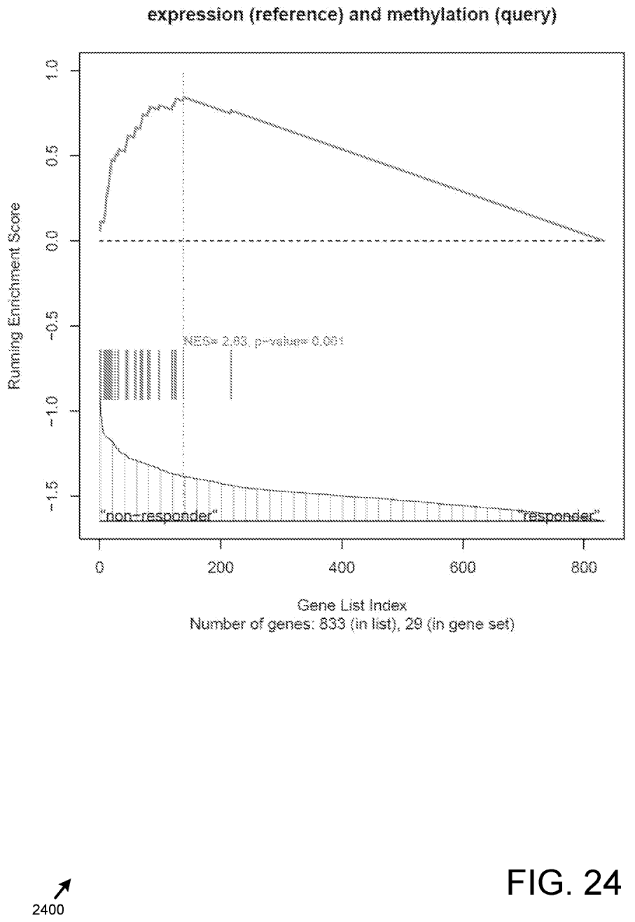

FIG. 23 is a box and whisker plot depicting p-value cutoff for query carboplatin-paclitaxel response composite methylation pathway signature (x-axis) and NESs from the corresponding GSEA comparisons between composite methylation and expression pathways signatures (y-axis).

P-value cut-off at 0.001 provides the most accurate average enrichment, and thus was selected for further analysis.

### Example 41—Example Detailed Implementation in R Programming Language: Transcriptomic and Epigenomic Pathway Integration

### Integrative Pathway Analysis: Integrative Transcriptomic-Epigenomic Pathway Analysis

Once query pathway set is established (as above), GSEA (e.g., from Broad) needs to be utilized so that composite expression pathway signature is used as a reference and composite methylation pathway signature as a query set (at p-value cut-o_0.001). Below is the GSEA output.

FIG. 24 is GSEA comparing carboplatin-paclitaxel response composite expression pathway signature (reference) and carboplatin-paclitaxel response composite methylation pathway signature (query, p<0.001), based on analysis in TCGA-LUAD patient cohort. NES and p-value were estimated using 1,000 pathway permutations.

Pathways from the GSEA leading edge (i.e., those affected on both transcriptomic and epigenomic levels) were then filtered to eliminate “parent-child” relationship, which resulted in 7 candidate pathways.

>seven_pathways

pathway

1 REACTOME_CHEMOKINE_RECEPTORS_BIND_CHEMOKINES

2 REACTOME_MRNA_SPLICING

3 REACTOME_G_ALPHAS_SIGNALLING_EVENTS

4 KEGG_INTESTINAL_IMMUNE_NETWORK_FOR_IGA_PRODUCTION

5 REACTOME_METABOLISM_OF_PROTEINS

6 KEGG_RNA_DEGRADATION

7 REACTOME_CELL_CYCLE_MITOTIC

### Example 42—Example Detailed Implementation in R Programming Language: Clinical Validation (Tang et al. Dataset)

To evaluate if these identified pathways can predict carboplatin-paclitaxel response in an independent, non-overlapping patient cohort, we considered patient cohort from Tang et al (Tang, H. et al. A 12-gene set predicts survival benefits from adjuvant chemotherapy in non-small cell lung cancer patients. Clinical cancer research: an official journal of the American Association for Cancer Research 19, 1577-1586 (2013)) with detailed description of the data available at GSE42127 https://www.ncbi.nlm.nih.gov/geo/query/acc.cgi?acc=GSE42127.

### Example 43—Example Detailed Implementation in R Programming Language: Clinical Validation (Tang et al. Dataset: Single Sample Analysis)

To evaluate activities of 7 pathways in each patient from the Tang et al cohort, we first scaled (i.e., z-scored on gene level) this patient data matrix (provided as tang_matrix_scaled R object) and considered sorted z-scores for each sample as a reference signature for signed and absolute valued single-sample GSEA pathway enrichment analysis (separately for expression and for methylation, as in section 2.2). Here, each single-sample signature (i.e., column in the tang_matrix_scaled matrix) is used as a reference and genes from each of 7 candidate pathways seven_pathways are used as a query gene set thus producing a pathway activity signature for each patient. The GSEA (i.e., from Broad) execution would be equivalent to the following pseudo code (or this code can be used directly if GSEA is downloaded locally):

> #code for single sample pathway enrichment analysis
> tang_single_sample_pathway_signatures <− matrix(0,7,39)
> pathways <− seven_pathways
> for(i in 1:length(pathways)){ #identified 7 candidate pathways
+ query <− pathway_genes(pathways[i])
+ for(j in 1:number_of_patients(tang_matrix_scaled)){ #number
of patients
+ r <− gsea(tang_matrix_scaled[,j ], query) # run GSEA
+ tang_single_sample_pathway_signatures[i,j] <− r.NES
+ }
+ }

Note that this analysis can be run for signed and absolute valued GSEAs, where NESs are integrated in the same way as described in the Transcriptomic and Epigenomic Pathway Integration sections (to define composite single-sample pathway signatures).

### Example 44—Example Detailed Implementation in R Programming Language: Clinical Validation (Tang et al. Dataset: Tang et al. Survival Analysis)

Next, t-Distributed Stochastic Neighbor Embedding (t-SNE) clustering (Maaten, L. v. d. and Hinton, G. Visualizing data using t-SNE. Journal of machine learning research 9, 2579-2605 (2008)) was employed on the tang_single_sample_pathway_signatures matrix to stratify patient into two groups, where clusters themselves alongside their clinical characteristics are provided in survival_data_Tang_luad, rows corresponding to sample (i.e., patient) names and columns corresponding to \time to event″, \event status″, and \cluster″.

Clinical details are described herein.

In order to evaluate if the patient groups significantly differ in their response to carboplatin-paclitaxel treatment, Kaplan-Meier Survival Analysis (Kaplan, E. L. and Meier, P. Nonparametric Estimation from Incomplete Observations. Journal of the American Statistical Association 53, 457-481 (1958)) was employed as follows:

> library(survival)
> library(survminer)
> tang_luad_surv <− survfit(Surv(survival_data_tang_luad$time,
+ survival_data_tang_luad$event) ~
+ survival_data_tang_luad$cluster, data = survival_data_tang_luad)
> tang_luad <−ggsurvplot(
+ tang_luad_surv,
+ data = survival_data_tang_luad,
+ pval = TRUE, # show p-value of log-rank test
+ xlim = c(0,2500), # present narrower X axis, but not affect survival
estimates
+ xlab = “Time in days”, # customize X axis label
+ break.time.by = 1000, # break X axis in time intervals
+ conf.int.style = “step”, # customize style of confidence intervals
+ legend.labs = c(“Group 1”, “Group 2”), palette = “Dark2”)
> print(tang_luad)

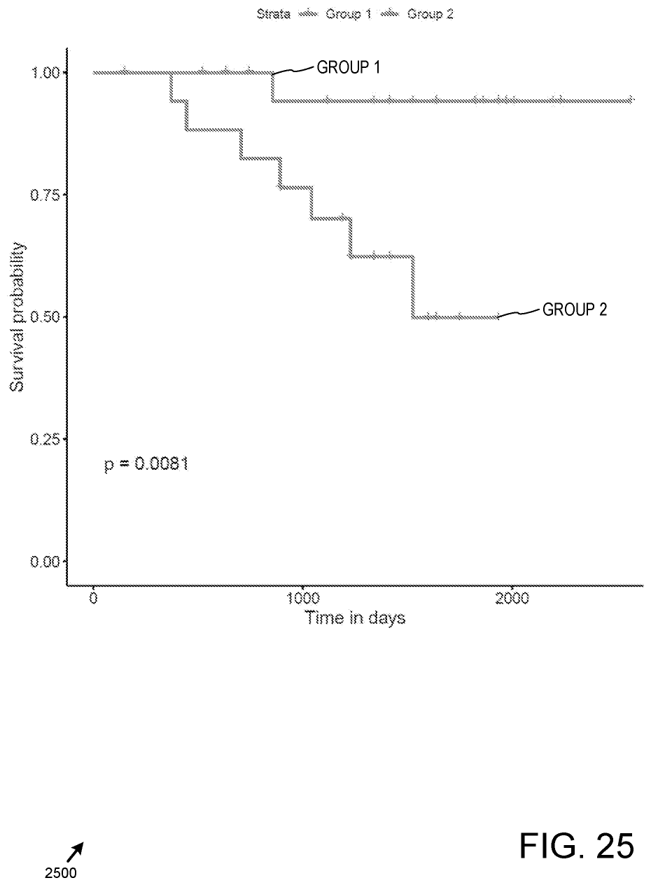

FIG. 25 is a graph of Kaplan-Meier survival analysis estimates difference in response to carboplatin-taxane between two patient groups. Log-rank p-value is indicated.

### Example 45—Example Detailed Implementation in R Programming Language: Clinical Validation (Random Model)

To evaluate non-randomness of this result, predictive ability of the candidate 7 pathways was compared to the predictive ability of 7 pathways selected at random, which demonstrated that ability of the candidate 7 pathways to predict carboplatin-paclitaxel response is highly non-random compared to 10,000 randomly selected pathways. A second random model was also employed, where the effect of selecting random patient groups was evaluated. For convenience, the results of the 10,000 times executing random model 1 are provided as random_density_plot_1 and result of the 10,000 times executing random model 2 as random_density_plot_2. Random model plots can be generated as follows:

> par(bty = ‘n’)
> plot(random_density_plot_1,col=“lightsteelblue4”,main=“”, xlim=c(0,11),
+ ylim=c(0,0.9), xlab = “-log2(log rank p-value)”)
> polygon(random_density_plot_1, col=adjustcolor(“lightsteelblue4”,
+ alpha.f=0.7),border=NA)
> lines(random_density_plot_2,col=“palegoldenrod”)
> polygon(random_density_plot_2, col=adjustcolor(“palegoldenrod”,
+ alpha.f=0.8),border=NA)
> arrows(6.96, 0.17, 6.96, 0, xpd = TRUE, lwd=2)
> text(7, .46,paste(“pvalue for random model 1 = ”,pvalue_for_random_model_1))
> text(7,.40,paste(“pvalue for random model 2 = ”,pvalue_for_random_model_2))
> text(6.5, .2,paste(“molecular pathways p-value”))

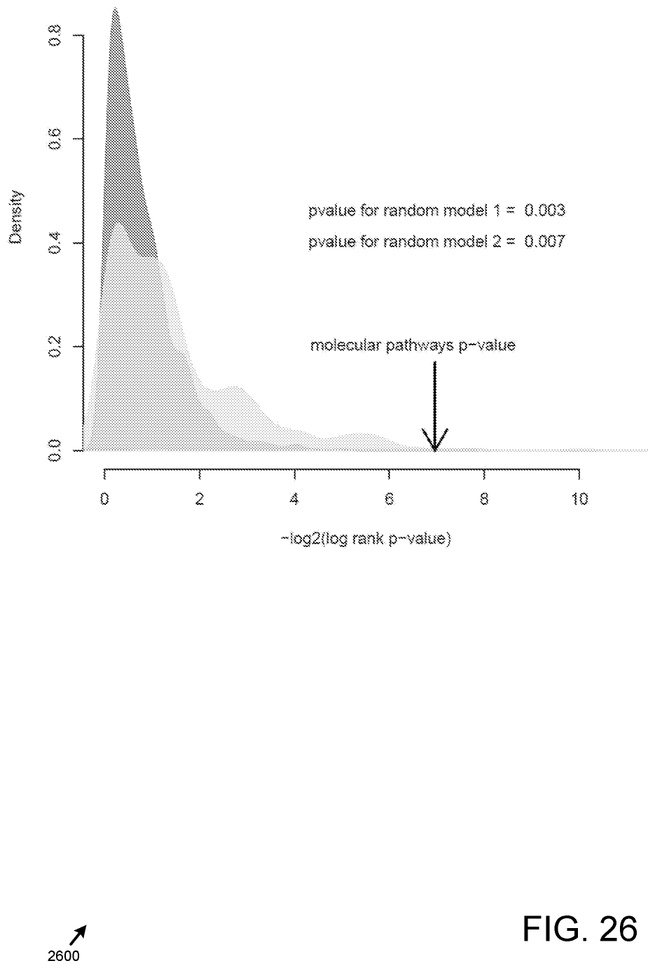

FIG. 26 is a graph of two random models that indicate non-random predictive ability of our model in the Tang et al. validation cohort: random model 1 (steel-blue) is defined based on 7 pathways selected at random, and random model 2 (goldenrod) is defined based on equally-sized patient groups selected at random.

### Example 46—Example Detailed Implementation in R Programming Language: Clinical Validation (Model Generalizability)

To test the generalizability of the implementation pathCHEMO, this approach was applied across additional chemotherapy regimens and cancer types: (1) cisplatin-vinorelbine response in lung adenocarcinoma (LUAD); (2) cisplatin-vinorelbine response in lung squamous cell carcinoma (LUSC); and (3) folinic acid, uorouracil, and oxaliplatin (i.e., FOLFOX) response in colorectal adenocarcinoma (COAD).

(1) For cisplatin-vinorelbine response in lung adenocarcinoma, patient cohort was used from Zhu et al. (Zhu, C. Q. et al. Prognostic and predictive gene signature for adjuvant chemotherapy in resected non-small-cell lung cancer. Journal of clinical oncology: official journal of the American Society of Clinical Oncology 28, 4417-4424 (2010)) with detailed description of the data available at GSE14814 https://www.ncbi.nlm.nih.gov/geo/query/acc.cgi?acc=GSE14814. R data object for Zhu et al. can be loaded as survival_data_zhu_luad and utilized for Kaplan-Meier survival analysis as follows:

> zhu_luad_surv <− survfit(Surv(survival_data_zhu_luad$time,
+ survival_data_zhu_luad$event) ~ survival_data_zhu_luad$cluster,
+ data = survival_data_zhu_luad)
> zhu_luad <−ggsurvplot(zhu_luad_surv,
+ data = survival_data_zhu_luad,
+ pval = TRUE, # show p-value of log-rank test
+ xlim = c(0,3500), # present narrower X axis, but not affect
+ xlab = “Time in days”, # customize X axis label
+ break.time.by = 1000, # break X axis in time intervals
+ conf.int.style = “step”, # customize style of confidence intervals
+ legend.labs = c(“Group 1”, “Group 2”), # change legend labels
+ palette = c(“aquamarine4”,‘darkorchid3’))
> print(zhu_luad)

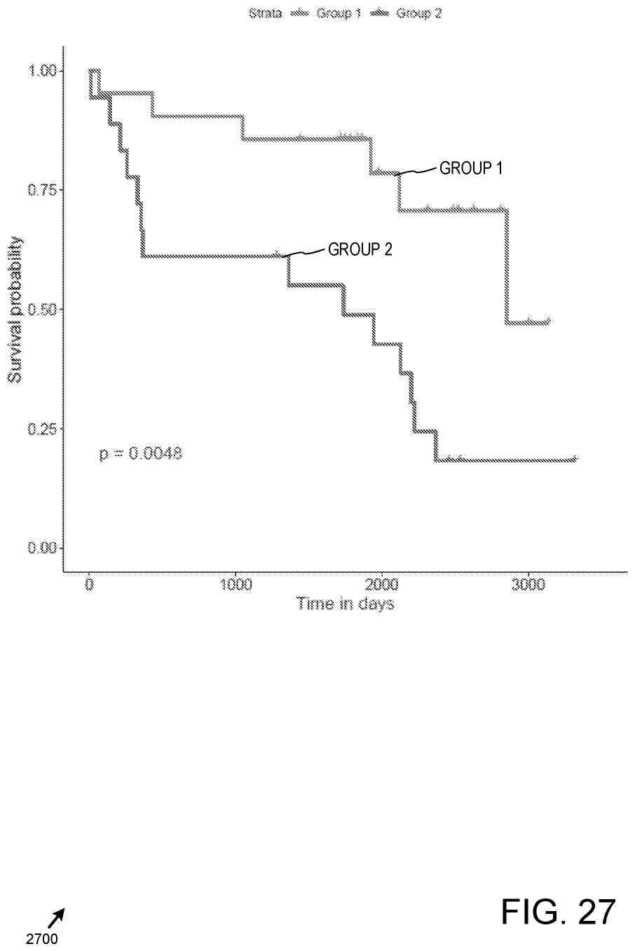

FIG. 27 is a graph of treatment related Kaplan-Meier survival analysis in cisplatin-vinorelbine treated lung adeno-carcinoma (LUAD) patients in the Zhu et al. patient cohort (n=39), demonstrating ability of identified candidate pathways to predict treatment response. Log rank p-value is indicated.

(2) For cisplatin-vinorelbine response in lung squamous cell carcinoma, we utilized patient cohort from Zhu et al (Zhu, C. Q. et al. Prognostic and predictive gene signature for adjuvant chemotherapy in resected non-small-cell lung cancer. Journal of clinical oncology: official journal of the American Society of Clinical Oncology 28, 4417-4424 (2010)) with detailed description of the data available at GSE14814 https://www.ncbi.nlm.nih.gov/geo/query/acc.cgi?acc=GSE14814. We are providing R data object as survival_data_zhu_lusc, which can be utilized for Kaplan-Meier survival analysis as follows:

> zhu_lusc_surv <− survfit(Surv(survival_data_zhu_lusc$time,
+ survival_data_zhu_lusc$event) ~ survival_data_zhu_lusc$cluster,
+ data = survival_data_zhu_lusc)
> zhu_lusc <−ggsurvplot(
+ zhu_lusc_surv,
+ data = survival_data_zhu_lusc,
+ pval = TRUE, # show p-value of log-rank test
+ xlim = c(0,3500), # present narrower X axis, but not affect
+ xlab = “Time in days”, # customize X axis label
+ break.time.by = 1000, # break X axis in time intervals
+ conf.int.style = “step”, # customize style of confidence intervals
+ legend.labs = c(“Group 1”, “Group 2”), # change legend labels
+ palette = c(“aquamarine4”,‘darkorchid3’))
> print(zhu_lusc)

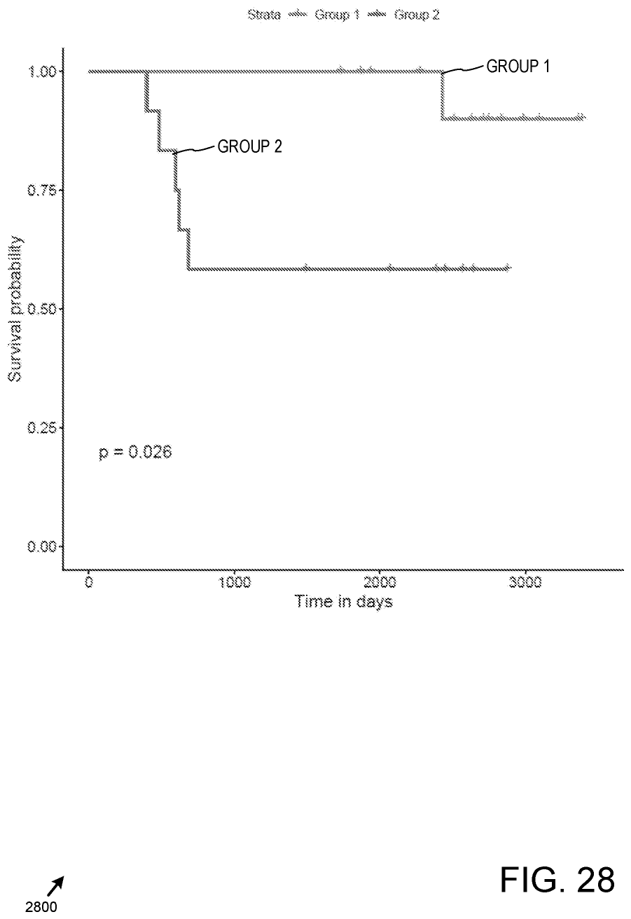

FIG. 28 is a graph of treatment related Kaplan-Meier survival analysis in cisplatin-vinorelbine treated lung squamous cell carcinoma (LUSC) patients in the Zhu et al. patient cohort (n=26), demonstrating ability of identified candidate pathways to predict treatment response. Log rank p-value is indicated.

(3) For FOLFOX response in colorectal adenocarcinoma we utilized patient cohort from Marisa et al (Marisa, L. et al. Gene expression classification of colon cancer into molecular subtypes: characterization, validation, and prognostic value. PLoS medicine 10, e1001453 (2013)) with detailed description of the data available at GSE39582 https://www.ncbi.nlm.nih.gov/geo/query/acc.cgi?acc=GSE39582. We are providing R data object as survival_data_marisa_coad, which can be used for Kaplan-Meier survival analysis as follows:

> marisa_coad_surv <− survfit(Surv(survival_data_marisa_coad$time,
+ survival_data_marisa_coad$event) ~
survival_data_marisa_coad$cluster,
+ data = survival_data_marisa_coad)
> marisa_coad <−ggsurvplot(marisa_coad_surv,
+ data = survival_data_marisa_coad, # data used to fit survival curves.
+ pval = TRUE, # show p-value of log-rank test.
+ xlim = c(0,3500), # present narrower X axis, but not affect
+ xlab = “Time in days”, # customize X axis label.
+ break.time.by = 1000, # break X axis in time intervals by 500.
+ conf.int.style = “step”, # customize style of confidence intervals
+ legend.labs = c(“Group 1”, “Group 2”), # change legend labels.,
+ palette = c(“aquamarine4”,‘darkorchid3’))
> print(marisa coad)

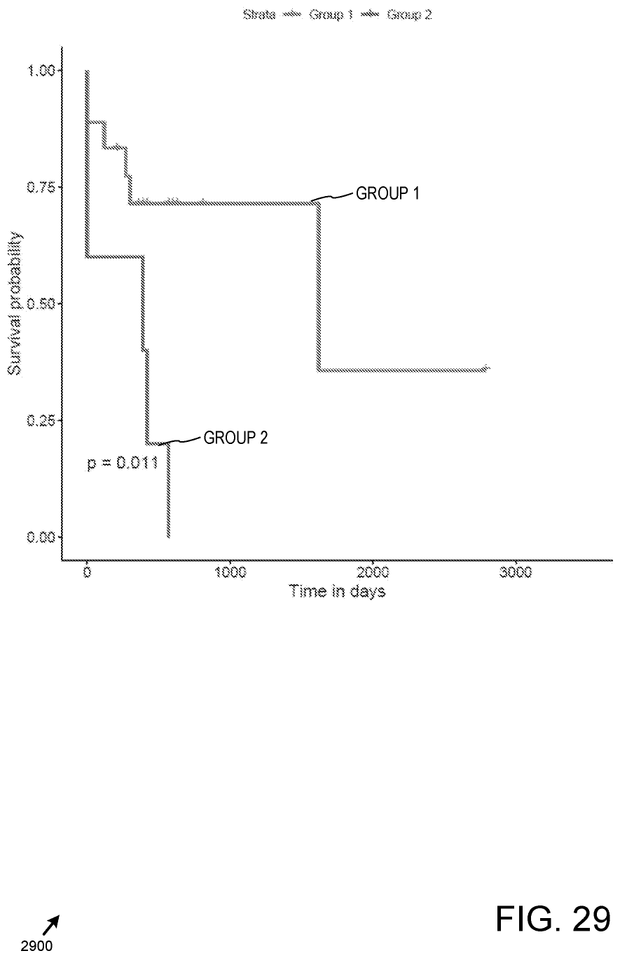

FIG. 29 is a graph of treatment related Kaplan-Meier survival analysis in FOLFOX (folinic acid, uorouracil, and oxaliplatin) treated colorectal adenocarcinoma (COAD) patients in the Marisa et al. patient cohort (n=23), demonstrating ability of identified candidate pathways to predict treatment response. Log rank p-value is indicated.

### Example 47—Example Explanations

In order to facilitate review of the various embodiments of the disclosure, the following explanations of specific terms are provided.

Actin-related protein ⅔ complex subunit 1A (ARPC1A): Also known as SOP2-LIKE (SOP2L), Epididymis Secretory Sperm Binding Protein 3, Epididymis Secretory Protein Li 307 3 (HEL-S-307 3), Epididymis Luminal Protein 68 3 (HEL-68 3), and Arc40 3 (for example, OMIM no. 604220), ARPC1A aids in regulating actin polymerization in cells and is involved in the actin Y pathway. ARPC1A nucleic acids and proteins are included. Exemplary ARPC1A DNA, mRNA, and proteins include GENBANK® sequences AY407874.1, NM_006409.4, and Q92747.2, respectively. Other ARPC1A molecules are possible. One of ordinary skill in the art can identify additional ARPC1A nucleic acid and protein sequences, including ARPC1A variant that retain biological activity (such as involvement in the actin Y pathway). In some examples, ARPC1A is upregulated (e.g., ARPC1A mRNA expression is increased) in a lung adenocarcinoma that will respond to cisplatin and vinorelbine combination chemotherapy, as compared to ARPC1A mRNA expression in a lung adenocarcinoma that will not respond to cisplatin and vinorelbine combination chemotherapy.

Administration/delivery: To provide or give a subject an agent or therapy by any effective route. Examples of agents include chemotherapy, surgery, radiation therapy, targeted therapy, immunotherapy, or palliative care. Administration includes acute and chronic administration as well as local and systemic administration. In some examples, administration of a therapeutic agent, such as chemotherapy, is by injection (e.g., intravenous, intramuscular, intraosseous, intratumoral, or intraperitoneal). In some examples, administration therapeutic agent, such as chemotherapy, is oral, transdermal, or rectal.

Adenocarcinoma: Carcinoma derived from glandular tissue or in which the tumor cells form recognizable glandular structures. Adenocarcinomas can be classified, according to the predominant pattern of cell arrangement as papillary, alveolar, etc., or according to a particular product of the cells, as mucinous adenocarcinoma. Adenocarcinomas arise in several tissues, including the kidney, breast, colon, cervix, esophagus, gastric, pancreas, prostate, and lung.

Animal: Living multi-cellular vertebrate organisms, a category that includes, for example, mammals and birds. The term mammal includes both human and non-human mammals. Similarly, the term “subject” includes both human and veterinary subjects.

C-C motif chemokine receptor 9 (CCR9): Also known as C-C chemokine receptor type 9 (CC-CKR-9), cluster of differentiation w199 (CDw199), G protein-coupled receptor 9-6 (GPR-9-6), and G protein-coupled receptor 28 (GPR28; for example, OMIM no. 604738), CCR9 is a member of the beta chemokine receptor family and is involved in the immune network for IgA production and chemokine receptor pathways. CCR9 nucleic acids and proteins are included. Exemplary CCR9 DNA, mRNA, and proteins include GENBANK® sequences AY242127.1, NM_031200.3, and AA092294.1, respectively. Other CCR9 molecules are possible. One of ordinary skill in the art can identify additional CCR9 nucleic acid and protein sequences, including CCR9 variant that retain biological activity (such as involvement in the immune network for IgA production and chemokine receptor pathways). In some examples, CCR9 is downregulated (e.g., expression of CCR9 mRNA is decreased) and methylation is increased (e.g., increased CCR9 DNA methylation) in a lung adenocarcinoma that will respond to carboplatin and paclitaxel combination chemotherapy, as compared to such expression and methylation in a lung adenocarcinoma that will not respond to carboplatin and paclitaxel combination chemotherapy.

C-C motif chemokine 11 (CCL11): Also known as small inducible cytokine subfamily A member 11 (SCYA11), eotaxin, and eosinophil chemotactic protein (for example, OMIM no. 601156, CCL11 recruits eosinophils and is involved in the cytokine-cytokine receptor interaction pathway. CCL11 nucleic acids and proteins are included. Exemplary CCL11 DNA, mRNA, and proteins include GENBANK® sequences EF064768.1, NM_002986.3, and CAG33702.1, respectively. Other CCL11 molecules are possible. One of ordinary skill in the art can identify additional CCL11 nucleic acid and protein sequences, including CCL11 variant that retain biological activity (such as involvement in the cytokine-cytokine receptor interaction pathway). In some examples, CCL11 is downregulated (e.g., CCL11 mRNA expression is decreased) and methylation is increased (e.g., increased CCL11 DNA methylation) in lung squamous cell carcinoma that will respond to cisplatin and vinorelbine combination chemotherapy as compared to such expression and methylation in a lung squamous cell carcinoma that will not respond to cisplatin and vinorelbine combination chemotherapy.

C-C motif chemokine ligand 22 (CCL22): Also known as small inducible cytokine subfamily a member 22 (SCYA22) and macrophage-derived chemokine (MDC; for example, OMIM no. 602957), CCL22 is secreted by dendritic cells and macrophages and is involved in the chemokine receptor pathway. CCL22 nucleic acids and proteins are included. Exemplary CCL22 DNA, mRNA, and proteins include GENBANK® sequences EF064764.1, NM_002990.5, and EAW82918.1, respectively. Other CCL22 molecules are possible. One of ordinary skill in the art can identify additional CCL22 nucleic acid and protein sequences, including CCL22 variant that retain biological activity (such as involvement in the chemokine receptor pathway). In some examples, CCL22 is downregulated (e.g., CCL22 mRNA expression is decreased) and methylation is increased (e.g., increased CCL22 DNA methylation) in a lung adenocarcinoma that will respond to carboplatin and paclitaxel combination chemotherapy, as compared to such expression and methylation in a lung adenocarcinoma that will not respond to carboplatin and paclitaxel combination chemotherapy.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, cancer cells, for example lung or colorectal cancer cells, are analyzed by the disclosed methods.

Cell division cycle 25B (CDC25B): CDC25B is a phosphatase that activates CDK1-cyclin B and is involved in the S phase pathway (for example, OMIM no. 116949). CDC25B nucleic acids and proteins are included. Exemplary CDC25B DNA, mRNA, and proteins include GENBANK® sequences AY494082.1, M81934.1, and P30305.2, respectively. Other CDC25B molecules are possible. One of ordinary skill in the art can identify additional human, mouse, and rat CDC25B nucleic acid and protein sequences, including CDC25B variant that retain biological activity (such as involvement in the S phase pathway). In some examples, CDC25B is upregulated (e.g., CDC25B mRNA expression is increased) and CDC25B methylation is decreased (e.g., decreased CDC25B DNA methylation) in a colon adenocarcinoma that will respond to FOLFOX combination chemotherapy, as compared to such expression and methylation in a colon adenocarcinoma that will not respond to FOLFOX combination chemotherapy.

Chaperonin-containing TCP1 subunit 4 (CCT4): Also known as CCT-delta (CCTD) and stimulator of TAR RNA-binding proteins (SRB; for example, OMIM no. 605142), CCT4 aids in protein folding as is involved in the protein metabolism pathway. CCT4 nucleic acids and proteins are included. Exemplary CCT4 DNA, mRNA, and proteins include GENBANK® sequences AC107081.5, NM_006430.4, and P50991.4, respectively. Other CCT4 molecules are possible. One of ordinary skill in the art can identify additional CCT4 nucleic acid and protein sequences, including CCT4 variant that retain biological activity (such as involvement in the protein metabolism pathway). In some examples, CCT4 is upregulated (e.g., CCT4 mRNA expression is increased) and CCT4 methylation is decreased (e.g., decreased CCT4 DNA methylation) in a lung adenocarcinoma carcinoma that will respond to carboplatin and paclitaxel combination chemotherapy, as compared to CCT4 expression and methylation in a lung adenocarcinoma that will not respond to carboplatin and paclitaxel combination chemotherapy.

Chemotherapeutic agent or Chemotherapy: Any chemical or biological agent with therapeutic usefulness in the treatment of diseases characterized by abnormal cell growth. Such diseases include tumors, neoplasms, and cancer. In one embodiment, a chemotherapeutic agent is an agent of use in treating lung cancer, such as lung adenocarcinoma or lung squamous cell carcinoma. In one embodiment, a chemotherapeutic agent is an agent of use in treating colorectal cancer, such as colorectal adenocarcinoma. In some examples, chemotherapeutic agents include carboplatin, paclitaxel, cisplatin, vinorelbine, folinic acid, fluorouracil, or oxaliplatin, in any combination together or with other agents. In some examples, the chemotherapeutic agents include a combination of carboplatin and paclitaxel, a combination of cisplatin and vinorelbine, and a combination of folinic acid, fluorouracil, and oxaliplatin. Exemplary chemotherapeutic agents are provided in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993, all incorporated herein by reference. Combination chemotherapy is the administration of more than one agent (such as more than one chemical chemotherapeutic agent) to treat cancer. Such a combination can be administered simultaneously, contemporaneously, or with a period of time in between.

Colorectal cancer: Also known as bowel or colon cancer, colorectal cancer includes cancer from the colon, rectum, or parts or the large intestine. Examples of colon cancer include adenocarcinoma, lymphoma, adenosquamous cell carcinoma, and squamous cell carcinoma. A variety of therapies can be used to treat colorectal cancer, including surgery, chemotherapy (such as folinic acid, fluorouracil, and oxaliplatin, for example, to treat colon adenocarcinoma), radiation therapy, targeted drug therapy, immunotherapy, and palliative care.

Control: A reference standard. In some embodiments, the control is a healthy subject. In other embodiments, the control is a subject with a cancer, such as a lung or colon cancer. In some embodiments, the control is a subject who responds positively to chemotherapy, such as a subject who does not develop resistance to chemotherapy. In other embodiments, the control is a subject who does not respond positively to chemotherapy, such as a subject who develops resistance to chemotherapy. In still other embodiments, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference.

Deoxythymidylate kinase (DTYMK): Also known as thymidylate kinase (TYMK) and CDC8 (for example OMIM no. 188345), DTYMK catalyzes phosphorylation of dTMP and is involved in the nucleotide metabolism pathway. TYMK nucleic acids and proteins are included. Exemplary TYMK DNA, mRNA, and proteins include GENBANK® sequences DQ052285.1, CR542015.1, and CAG46783.1, respectively. Other TYMK molecules are possible. One of ordinary skill in the art can identify additional TYMK nucleic acid and protein sequences, including TYMK variant that retain biological activity (such as involvement in the nucleotide metabolism pathway). In some examples, DTYMK is upregulated (e.g., DTYMK mRNA expression is increased) in a lung adenocarcinoma that will respond to cisplatin and vinorelbine combination chemotherapy, as compared to DTYMK mRNA expression in a lung adenocarcinoma that will not respond to cisplatin and vinorelbine combination chemotherapy.

Detect: To determine if an agent (such as a signal; particular nucleotide; amino acid; nucleic acid molecule and/or nucleotide modification, such as a methylated nucleotide; mRNA; or protein) is present or absent. In some examples, detection can include further quantification. For example, use of the disclosed methods in particular examples permits detection of nucleic acid expression (e.g., mRNA expression) or nucleic acid modification (such as DNA methylation) in a sample.

Differential Expression: A nucleic acid molecule is differentially expressed when the amount of one or more of its expression products (e.g., transcript, such as mRNA, and/or protein) is higher or lower in one sample (such as a test lung or colorectal cancer sample) as compared to another sample (such as a control lung or colorectal cancer sample). Detecting differential expression can include measuring a change in gene (such as by measuring mRNA) or protein expression.

Differential methylation: A nucleic acid molecule is differentially methylated when the amount of methylated nucleotides in the gene (such as the gene body) or sequences associated with gene transcription (such as promoters, for example, in CpG islands of promoters) is higher or lower in one sample (such as a test lung or colorectal cancer sample) as compared to another sample (such as a control lung or colorectal cancer sample). Detecting differential methylation can include measuring methylation using a bisulfate conversion assay or any other method of detecting DNA methylation (e.g., Levenson et al., Expert Rev Mol Diagn, 10(4): 481-488, 2010, incorporated herein by reference in its entirety).

Excision repair cross-complementation group 1 (ERCC1): Also known as Excision Repair Cross-Complementing Rodent Repair Deficiency Complementation Group 1, COFS4, RAD10, and UV20 (for example, OMIM no. 126380), ERCC1 is involved in the DNA repair pathway. ERCC1 nucleic acids and proteins are included. Exemplary ERCC1 DNA, mRNA, and proteins include GENBANK® sequences AF512555.1, AF001925.1, and P07992.1, respectively. Other ERCC1 molecules are possible. One of ordinary skill in the art can identify additional ERCC1 nucleic acid and protein sequences, including ERCC1 variant that retain biological activity (such as involvement in the DNA repair pathway). In some examples, ERCC1 expression is downregulated (e.g., ERCC1 mRNA expression is decreased) in a lung squamous cell carcinoma that will respond to cisplatin and vinorelbine combination chemotherapy, as compared to ERCC1 expression in a lung squamous cell carcinoma that will not respond to cisplatin and vinorelbine combination chemotherapy.

Expression: Translation of a nucleic acid into a peptide or protein. Peptides or proteins may be expressed and remain intracellular, become a component of the cell surface membrane, or be secreted into the extracellular matrix or medium.

Fibroblast growth factor receptor 1 oncogene partner (FGFR1OP): Also known as FGFR1 oncogene partner (FOP; OMIM no. 605392), FGFR1OP plays a role in cell proliferation and differentiation and is involved in the mitotic cell cycle pathway. FGFR1OP nucleic acids and proteins are included. Exemplary FGFR1OP DNA, mRNA, and proteins include DQ030392.1, BC037785.1, and AAH11902.1, respectively. Other FGFR1OP molecules are possible. One of ordinary skill in the art can identify additional FGFR1OP nucleic acid and protein sequences, including FGFR1OP variant that retain biological activity (such as involvement in the mitotic cell cycle pathway). In some examples, expression of FGFR1OP is downregulated (e.g., FGFR1OP mRNA expression is decreased) and methylation decreased (e.g., FGFR1OP DNA methylation is decreased) in a lung adenocarcinoma carcinoma that will respond to carboplatin and paclitaxel combination chemotherapy as compared to FGFR1OP expression and methylation in a lung adenocarcinoma that will not respond to carboplatin and paclitaxel combination chemotherapy.

Gamma-aminobutyric acid receptor alpha-1 (GABRA1): Also known as GABA-A receptor, alpha-1 polypeptide, EIEE19, ECA4, EJM5, and EJM, GABRA1 is an inhibitory neurotransmitter and is involved in the neuroactive ligand-receptor interaction pathway. GABRA1 nucleic acids and proteins are included. Exemplary GABRA1 DNA, mRNA, and proteins include NG_011548.1, NM_000806.5, and AAH30696.1, respectively. Other GABRA1 molecules are possible. One of ordinary skill in the art can identify additional GABRA1 nucleic acid and protein sequences, including GABRA1 variant that retain biological activity (such as involvement in the neuroactive ligand-receptor interaction pathway). In some examples, methylation of GABRA1 is increased (e.g., increased GABRA1 DNA methylation) in a lung squamous cell carcinoma that will respond to cisplatin and vinorelbine combination chemotherapy, as compared to GABRA1 methylation in a lung squamous cell carcinoma that will not respond to cisplatin and vinorelbine combination chemotherapy.

Inhibiting or treating a disease: Inhibiting the full development of a disease or condition, for example, in a subject who is at risk for a disease, such as a subject with cancer, for example, lung or colon cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

LSM7: Also known as YNL147 W, LSM7 homolog U6 small nuclear RNA and mRNA degradation associated (for example, OMIM no. 607287), LSM7 forms an oligomer that interacts with RNA to form a protein-RNA complex and is involved in the RNA degradation pathway. LSM7 nucleic acids and proteins are included. Exemplary LSM7 DNA, mRNA, and proteins include AF182293, NM_016199.3, and NP_057283.1, respectively. Other LSM7 molecules are possible. One of ordinary skill in the art can identify additional LSM7 nucleic acid and protein sequences, including LSM7 variant that retain biological activity (such as involvement in the RNA degradation pathway). In some examples, methylation of LSM7 is decreased (e.g., LSM7 DNA methylation is decreased) in a lung adenocarcinoma carcinoma that will respond to carboplatin and paclitaxel combination chemotherapy as compared to LSM7 DNA methylation in a lung adenocarcinoma that will not respond to carboplatin and paclitaxel combination chemotherapy.

Lung cancer: A cancer of the lung tissue. Lung cancer can be small-cell or non-small cell lung cancer. Examples of non-small cell carcinoma include adenocarcinoma, squamous-cell carcinoma, and large-cell carcinoma. A variety of therapies can be administered to treat or inhibit lung cancers, such as chemotherapy (for example, carboplatin, paclitaxel, cisplatin, vinorelbine, or a combination thereof can be used for treatment, such as carboplatin and paclitaxel, for example to treat lung adenocarcinoma, or cisplatin and vinorelbine, for example, to treat lung adenocarcinoma or lung squamous cell carcinoma), surgery, radiation therapy, targeted therapy, immunotherapy, and palliative care.

Methylation: Methylation of DNA can alter the activity of the DNA without changing the sequence. Two bases in DNA can be methylated, cytosine and adenine. Methylation can be used to either express or repress genes; often methylation of CpG islands in promoters are associated with gene repression, while methylation of a gene body is often associated with high levels of gene transcription.

Myosin light chain kinase 3 (MYLK3): Also known as cardiac MLCK, MYLK3 plays a role in regulating cardiovascular function and is involved in the calcium signaling pathway. MYLK3 nucleic acids and proteins are included. Exemplary MYLK3 DNA, mRNA, and proteins include HF584427.1, AJ247087.1, and Q32MK0.3, respectively. Other MYLK3 molecules are possible. One of ordinary skill in the art can identify additional MYLK3 nucleic acid and protein sequences, including MYLK3 variant that retain biological activity (such as involvement in the calcium signaling pathway). In some examples, expression of MYLK3 is downregulated (e.g., MYLK3 mRNA expression is decreased) and methylation of MYLK3 is increased (e.g., MYLK3 DNA methylation is increased) in a subject with colon adenocarcinoma that will respond to FOLFOX combination chemotherapy, as compared to MYLK3 expression and methylation in a colon adenocarcinoma that will not respond to FOLFOX combination chemotherapy.

Phosphodiesterase 7A (PDE7A): Also known as high affinity CAMP-specific 3′,5′-cyclic phosphodiesterase 7A, human complement of yeast PDE1/PDE2 (HCP1; for example, OMIM no. 171885), PDE7A regulates concentrations of cyclic nucleotides and are involved in the G alpha signaling pathway. PDE7A nucleic acids and proteins are included. Exemplary PDE7A DNA, mRNA, and proteins include NG_029614.1, L12052.1, and Q13946.2, respectively. Other PDE7A molecules are possible. One of ordinary skill in the art can identify additional PDE7A nucleic acid and protein sequences, including PDE7A variant that retain biological activity (such as involvement in the G alpha signaling pathway). In some examples, PDE7A is downregulated (e.g., PDE7A mRNA expression is decreased) in a lung adenocarcinoma that will respond to carboplatin and paclitaxel combination chemotherapy, as compared to PDE7A expression in a lung adenocarcinoma that will not respond to carboplatin and paclitaxel combination chemotherapy.

Pre-mRNA processing factor 6 (PRPF6): Also known as PRP6, androgen receptor n-terminal domain-transactivating protein 1, ANT1, TOM, and chromosome 20 open reading frame 14 (C200RF14; for example, OMIM no. 613979), PRPF6 binds androgen receptor and is involved in the processing of capped intron containing pre-mRNA pathway. PRPF6 nucleic acids and proteins are included. Exemplary PRPF6 DNA, mRNA, and proteins include NG_029719.1, NM_012469.4, and O94906.1, respectively. Other PRPF6 molecules are possible. One of ordinary skill in the art can identify additional PRPF6 nucleic acid and protein sequences, including PRPF6 variants that retain biological activity (such as involvement in the processing of capped intron containing pre-mRNA pathway). In some examples, PRPF6 is upregulated (e.g., PRPF6 mRNA expression is increased) in a colon adenocarcinoma that will respond to FOLFOX combination chemotherapy, as compared to PRPF6 expression in a colon adenocarcinoma that will not respond to FOLFOX combination chemotherapy.

Prefoldin subunit 1 (PFDN1): PFDN1 (for example, OMIM no. 604897) aids in binding and stabilizing newly synthesized polypeptides and is involved in the protein metabolism pathway. PFDN1 nucleic acids and proteins are included. Exemplary PFDN1 DNA, mRNA, and proteins include AY421527.1, NM_002622.5, and NP_002613.2, respectively. Other PFDN1 molecules are possible. One of ordinary skill in the art can identify additional PFDN1 nucleic acid and protein sequences, including PFDN1 variant that retain biological activity (such as involvement in the protein metabolism pathway). In some examples, methylation of PFDN1 is decreased (e.g., PFDN1 DNA methylation is decreased) in a colon adenocarcinoma that will respond to FOLFOX combination chemotherapy, as compared to PFDN1 methylation in a colon adenocarcinoma that will not respond to FOLFOX combination chemotherapy.

Ribosomal protein lateral stalk subunit P2 (RPLP2): Also known as 60S acidic ribosomal protein P2, large ribosomal subunit protein P2, acidic ribosomal phosphoprotein P2, P2, LP2, renal carcinoma antigen NY-REN-44, RPLP2 is a part of the 60S subunit and is involved in the ribosome pathway. RPLP2 nucleic acids and proteins are included. Exemplary RPLP2 DNA, mRNA, and proteins include DQ036650.1, NM_001004.4, and CAG47008.1, respectively. Other RPLP2 molecules are possible. One of ordinary skill in the art can identify additional RPLP2 nucleic acid and protein sequences, including RPLP2 variants that retain biological activity (such as involvement in the ribosome pathway). In some examples, RPLP2 is upregulated (e.g., RPLP2 mRNA expression is increased) and RPLP2 methylation decreased (e.g., methylation of RPLP2 DNA is decreased) in a subject with lung adenocarcinoma that will respond to cisplatin and vinorelbine combination chemotherapy, as compared to RPLP2 expression and methylation in a lung adenocarcinoma that will not respond to cisplatin and vinorelbine combination chemotherapy.

Ribosomal protein L14 (RPL14): Also known as 60S Ribosomal Protein L14, Large Ribosomal Subunit Protein EL14, CAG-ISL 7, CTG-B33, HRL14, and L14, RPL14 is a part of the 60S subunit and is involved in the translation pathway. RPL14 is a subunit for RNA polymerase and is involved in the RNA splicing pathway. RPL14 nucleic acids and proteins are included. Exemplary RPL14 DNA, mRNA, and proteins includes AB061822.1, BC009294.2, and AAH71913.1, respectively. Other RPL14 molecules are possible. One of ordinary skill in the art can identify additional RPL14 nucleic acid and protein sequences, including RPL14 variants that retain biological activity (such as involvement in the RNA splicing pathway). In some examples, methylation of RPL14 is decreased (e.g., methylation of RPL14 DNA is decreased) in a lung squamous cell carcinoma that will respond to cisplatin and vinorelbine combination chemotherapy, as compared to RPL14 methylation in a lung squamous cell carcinoma that will not respond to cisplatin and vinorelbine combination chemotherapy.

RNA polymerase II subunit C (POLR2C): Also known as DNA-directed RNA polymerase II subunit 3 (RPB3), DNA-Directed RNA Polymerase II 33 KDa Polypeptide (RPB33), RPB31, hRPB33, and hsRPB3 (OMIM no. 180663), POLR2C is a subunit for RNA polymerase and is involved in the RNA splicing pathway. POLR2C nucleic acids and proteins are included. Exemplary POLR2C DNA, mRNA, and proteins include DQ032841.1, CR542041.1, and CAG46838.1, respectively. Other POLR2C molecules are possible. One of ordinary skill in the art can identify additional POLR2C nucleic acid and protein sequences, including POLR2C variants that retain biological activity (such as involvement in the RNA splicing pathway). In some examples, POLR2C is upregulated (e.g., POLR2C mRNA expression is increased) and methylation of POLR2C is decreased (e.g., methylation of POLR2C DNA is decreased) in a lung adenocarcinoma that will respond to carboplatin and paclitaxel combination chemotherapy, as compared to such expression and methylation in a lung adenocarcinoma that will not respond to carboplatin and paclitaxel combination chemotherapy.

Sample or biological sample: A sample of biological material obtained from a subject, which can include cells, proteins, and/or nucleic acid molecules (such as DNA and/or RNA, such as mRNA). Biological samples include all clinical samples useful for detection of disease, such as cancer, in subjects. Appropriate samples include any conventional biological samples, including clinical samples obtained from a human or veterinary subject. Exemplary samples include, without limitation, cancer samples (such as from surgery, tissue biopsy, tissue sections, or autopsy), cells, cell lysates, blood smears, cytocentrifuge preparations, cytology smears, bodily fluids (e.g., blood, plasma, serum, stool/feces, saliva, sputum, urine, bronchoalveolar lavage, semen, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as lung or colorectal cancer). In a specific example, the sample is a lung cancer sample. In another specific example, the sample is a colorectal cancer sample.

Solute carrier family 44 member 4 (SLC44A4): Also known as choline transporter-like protein 4 (CTL4), thiamine pyrophosphate transporter (TPPT), TPP transporter, chromosome 6 open reading frame 29 (C6ORF29), and testicular tissue protein Li 48 (for example, OMIM no. 606107), SLC44A4 aids in supplying choline to cells and is involved in the solute carrier (SLC)-mediated transmembrane transport pathway. SLC44A4 nucleic acids and proteins are included. Exemplary SLC44A4 DNA, mRNA, and proteins include KY500657.2, NM_200413.1, and AQY77128.1, respectively. Other SLC44A4 molecules are possible. One of ordinary skill in the art can identify additional SLC44A4 nucleic acid and protein sequences, including SLC44A4 variants that retain biological activity (such as involvement in the solute carrier (SLC)-mediated transmembrane transport pathway). In some examples, methylation of SLC44A4 is decreased (e.g., SLC44A4 DNA methylation is decreased) in a lung squamous cell carcinoma that will respond to cisplatin and vinorelbine combination chemotherapy as compared to a lung squamous cell carcinoma that will not respond to cisplatin and vinorelbine combination chemotherapy.

Splicing factor 3b subunit 3 (SF3B3): Also known as spliceosome-associated protein 130 kd (SAP130; for example OMIM no. 605592), SF3B3 is a component of small nuclear ribonucleoprotein and spliceosome complexes and is involved in the elongation and processing of capped transcripts pathway. SF3B3 nucleic acids and proteins are included. Exemplary SF3B3 DNA includes NG_046937.1, BC068974.1, and Q15393.4, respectively. Other SF3B3 molecules are possible. One of ordinary skill in the art can identify additional SF3B3 nucleic acid and protein sequences, including SF3B3 variants that retain biological activity (such as involvement in the elongation and processing of capped transcripts pathway). In some examples, SF3B3 is upregulated (e.g., SF3B3 mRNA expression is increased) in a colon adenocarcinoma that will respond to FOLFOX combination chemotherapy, as compared to SF3B3 expression in a colon adenocarcinoma that will not respond to FOLFOX combination chemotherapy.

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In one example, the subject treated and/or analyzed with the disclosed methods has cancer, such as lung or colorectal cancer. In some examples, the subject responds positively to chemotherapy, such as a subject who does not develop resistance to chemotherapy.

Therapeutically effective amount: The amount of an active ingredient (such as a chemotherapeutic agent) that is sufficient to effect treatment when administered to a mammal in need of such treatment, such as treatment of a cancer. The therapeutically effective amount will vary depending upon the subject and disease condition being treated, the weight and age of the subject, the severity of the disease condition, the manner of administration and the like, which can readily be determined by a prescribing physician.

Treating, treatment, and therapy: Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's sensorimotor function. The treatment may be assessed by objective or subjective parameters; including the results of a physical examination, neurological examination, or psychiatric evaluations. For example, treatment of a cancer can include decreasing the size, volume, or weight of a cancer, decrease the number, size, volume, or weight of metastases, or combinations thereof.

Tumor, neoplasia, malignancy or cancer: A neoplasm is an abnormal growth of tissue or cells which results from excessive cell division. Neoplastic growth can produce a tumor. The amount of a tumor in an individual is the “tumor burden”, which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer. Exemplary tumors, such as cancers, that can be analyzed and treated with the disclosed methods include carcinomas of the lung (such as squamous cell carcinoma and adenocarcinoma) and colorectal adenocarcinoma.

U2 small nuclear RNA auxiliary factor 1 (U2AF1): Also known as U2 small nuclear ribonucleoprotein auxiliary factor 35-kd subunit (U2AF35), Splicing Factor U2AF 35 kd Subunit, U2AFBP, U2AF35, RNU2AF1, FP793, and RN, U2AF1 plays a role in RNA splicing and is involved in the transport of mature mRNA derived from an intron-containing transcript pathway. U2AF1 nucleic acids and proteins are included. Exemplary U2AF1 DNA, mRNA, and proteins include NG_029455.1, BC005915.1, and Q01081.3, respectively. Other U2AF1 molecules are possible. One of ordinary skill in the art can identify additional U2AF1 nucleic acid and protein sequences, including U2AF1 variants that retain biological activity (such as involvement in the intron-containing transcript pathway). In some examples, U2AF1 is downregulated (e.g., U2AF1 mRNA expression is decreased) in a lung squamous cell carcinoma that will respond to cisplatin and vinorelbine combination chemotherapy, as compared to U2AF1 expression in a lung squamous cell carcinoma that will not respond to cisplatin and vinorelbine combination chemotherapy.

### Example 48—Example Computing System

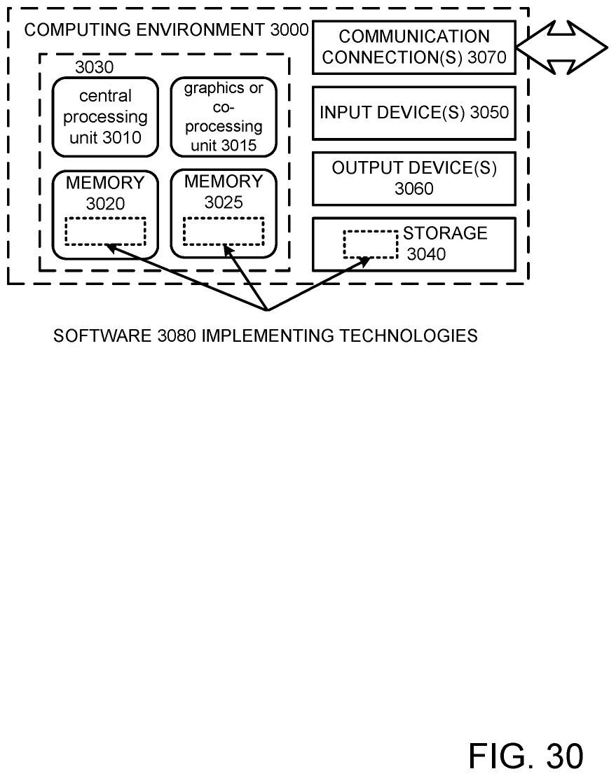

FIG. 30 illustrates a generalized example of a suitable computing system 3000 in which any of the described technologies may be implemented. The computing system 3000 is not intended to suggest any limitation as to scope of use or functionality, as the innovations may be implemented in diverse computing systems, including special-purpose computing systems. In practice, a computing system can comprise multiple networked instances of the illustrated computing system.

With reference to FIG. 30, the computing system 3000 includes one or more processing units 3010, 3015 and memory 3020, 3025. In FIG. 30, this basic configuration 3030 is included within a dashed line. The processing units 3010, 3015 execute computer-executable instructions. A processing unit can be a central processing unit (CPU), processor in an application-specific integrated circuit (ASIC), or any other type of processor. In a multi-processing system, multiple processing units execute computer-executable instructions to increase processing power. For example, FIG. 30 shows a central processing unit 3010 as well as a graphics processing unit or co-processing unit 3015. The tangible memory 3020, 3025 may be volatile memory (e.g., registers, cache, RAM), non-volatile memory (e.g., ROM, EEPROM, flash memory, etc.), or some combination of the two, accessible by the processing unit(s). The memory 3020, 3025 stores software 3080 implementing one or more innovations described herein, in the form of computer-executable instructions suitable for execution by the processing unit(s).

A computing system may have additional features. For example, the computing system 3000 includes storage 3040, one or more input devices 3050, one or more output devices 3060, and one or more communication connections 3070. An interconnection mechanism (not shown) such as a bus, controller, or network interconnects the components of the computing system 3000. Typically, operating system software (not shown) provides an operating environment for other software executing in the computing system 3000, and coordinates activities of the components of the computing system 3000.

The tangible storage 3040 may be removable or non-removable, and includes magnetic disks, magnetic tapes or cassettes, CD-ROMs, DVDs, or any other medium which can be used to store information in a non-transitory way and which can be accessed within the computing system 100. The storage 3040 stores instructions for the software 3080 implementing one or more innovations described herein.

The input device(s) 3050 may be a touch input device such as a keyboard, mouse, pen, or trackball, a voice input device, a scanning device, or another device that provides input to the computing system 3000. For video encoding, the input device(s) 3050 may be a camera, video card, TV tuner card, or similar device that accepts video input in analog or digital form, or a CD-ROM or CD-RW that reads video samples into the computing system 3000. The output device(s) 160 may be a display, printer, speaker, CD-writer, or another device that provides output from the computing system 3000.

The communication connection(s) 3070 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor (e.g., that is ultimately implemented on a hardware processor). Generally, program modules include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system.

For the sake of presentation, the detailed description uses terms like “determine” and “use” to describe computer operations in a computing system. These terms are high-level abstractions for operations performed by a computer and should not be confused with acts performed by a human being. The actual computer operations corresponding to these terms vary depending on implementation.

### Example 49—Example Cloud Computing Environment

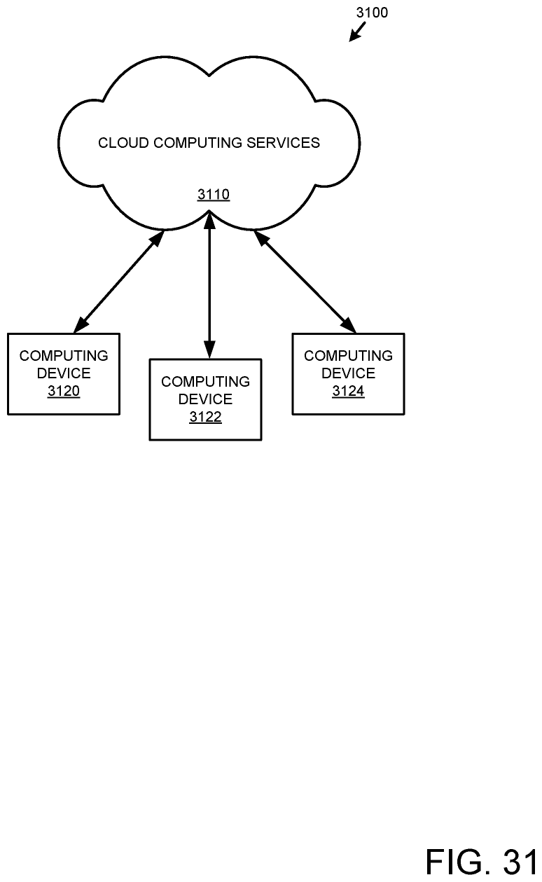

FIG. 31 depicts an example cloud computing environment 3100 in which the described technologies can be implemented, including, e.g., the systems of the drawings described herein. The cloud computing environment 3100 comprises cloud computing services 3110. The cloud computing services 3110 can comprise various types of cloud computing resources, such as computer servers, data storage repositories, networking resources, etc. The cloud computing services 3110 can be centrally located (e.g., provided by a data center of a business or organization) or distributed (e.g., provided by various computing resources located at different locations, such as different data centers and/or located in different cities or countries).

The cloud computing services 3110 are utilized by various types of computing devices (e.g., client computing devices), such as computing devices 3120, 3122, and 3124. For example, the computing devices (e.g., 3120, 3122, and 3124) can be computers (e.g., desktop or laptop computers), mobile devices (e.g., tablet computers or smart phones), or other types of computing devices. For example, the computing devices (e.g., 3120, 3122, and 3124) can utilize the cloud computing services 3110 to perform computing operations (e.g., data processing, data storage, and the like).

In practice, cloud-based, on-premises-based, or hybrid scenarios can be supported.

### Example 50—Example Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

### Example 51—Example Implementations

Although the operations of some of the disclosed methods are described in a particular, sequential order for convenient presentation, such manner of description encompasses rearrangement, unless a particular ordering is required by specific language set forth herein. For example, operations described sequentially can in some cases be rearranged or performed concurrently.

### Example 52—Example Computer-Executable Implementation

Any of the methods described herein can be performed by computer-executable instructions (e.g., causing a computing system to perform the method, when executed) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

Such acts of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

In any of the technologies described herein, the illustrated actions can be described from alternative perspectives while still implementing the technologies. For example, “receiving” can also be described as “sending” for a different perspective.

### Example 53—Further Embodiments

Any of the following can be implemented.

Clause 1. A computer-implemented method of identifying treatment-response biomarkers, comprising:

(i) receiving genomic and epigenomic datasets for at least two subjects, wherein the at least two subjects have different treatment-response phenotypes;

(ii) identifying differential genomic and epigenomic datapoints in the datasets, wherein the identifying generates at least one differential genomic signature for the treatment-response phenotypes and at least one differential epigenomic signature for the treatment-response phenotypes;

(iii) determining biological pathways enriched in the at least one differential genomic signature and the at least one differential epigenomic signature, wherein the determining generates at least one genomic pathway signature and at least one epigenomic pathway signature; and

(iv) selecting biological pathways enriched between the at least one genomic pathway signature and the at least one epigenomic pathway signature, wherein the selecting generates a comprehensive pathway signature for the treatment-response phenotypes.

Clause 2. The method of Clause 1, wherein:

the at least one differential genomic signature for the treatment-response phenotypes comprises a signed differential genomic signature for the treatment-response phenotypes and/or an absolute valued differential genomic signature for the treatment-response phenotypes; and/or

the at least one differential epigenomic signature for the treatment-response phenotypes comprises a signed differential epigenomic signature for the treatment-response phenotypes and/or an absolute valued differential epigenomic signature for the treatment-response phenotypes.

Clause 3. The method of Clause 2, wherein the at least one genomic pathway signature and at least one epigenomic pathway signature comprises:

a signed genomic pathway signature and/or an absolute valued genomic pathway signature; and/or

a signed epigenomic pathway signature and/or an absolute valued epigenomic pathway signature.

Clause 4. The method of Clause 3, wherein the at least one genomic pathway signature and at least one epigenomic pathway signature comprises:

a signed genomic pathway signature and an absolute valued genomic pathway signature; and/or

a signed epigenomic pathway signature and an absolute valued epigenomic pathway signature.

Clause 5. The method of Clause 4, further comprising:

combining the signed genomic pathway signature and the absolute valued genomic pathway signature, wherein the combining generates a composite genomic pathway signature; and/or

combining the signed epigenomic pathway signature and the absolute valued epigenomic pathway signature, wherein the combining generates a composite epigenomic pathway signature.

Clause 6. The method of Clause 5, wherein combining comprises selecting for a highest pathway enrichment.

Clause 7. The method of any one of Clauses 1-6, wherein the genomic dataset comprises gene expression datapoints.

Clause 8. The method of any one of Clauses 1-7, wherein the epigenomic dataset comprises gene methylation datapoints.

Clause 9. The method of any one of Clauses 1-8, wherein the at least 2 subjects comprise subjects that have cancer, and the different treatment-response phenotypes comprise a positive response to chemotherapy treatment and a negative response to chemotherapy treatment.

Clause 10. The method of any one of Clauses 1-9, wherein the at least one differential genomic signature for the treatment-response phenotypes and/or the at least one differential epigenomic signature for the treatment-response phenotypes comprises genes ranked based on level of differentiation.

Clause 11. The method of any one of Clauses 5-10, wherein the selecting biological pathways comprises removing pathways from the composite genomic pathway signature and the composite epigenomic pathway signature that are not associated with both differential genomic and epigenomic datapoints.

Clause 12. The method of any one of Clauses 5-11, wherein selecting biological pathways comprises removing pathways from the composite genomic pathway signature and the composite epigenomic pathway signature that are not enriched with a p value of less than 0.001.

Clause 13. The method of any one of Clauses 1-12, wherein the datapoints are normalized before identifying differential genomic and epigenomic datapoints in the datasets.

Clause 14. The method of any one of Clauses 1-13, further comprising validating clinical significance, non-randomness, and/or accuracy of the comprehensive pathway signature.

Clause 15. The method of Clause 14, wherein validating the clinical significance comprises:

receiving genomic and epigenomic datasets for a group of validation subjects, wherein treatment-response phenotypes are known;

identifying differential genomic and epigenomic datapoints in the datasets, wherein the identifying generates at least one single-sample signature for each validation subject in the group;

determining enrichment of biological pathways from the comprehensive pathway signature in the at least one single-sample signature for each validation subject in the group, wherein the determining generates a pathway activity signature for each validation subject;

clustering the validation subjects in the group into treatment-response clusters based on the pathway activity signature for each validation subject; and

comparing the treatment response clusters with the known treatment-response phenotypes.

Clause 16. The method of Clause 15, wherein comparing the treatment response clusters with the known treatment-response phenotypes comprises statistically analyzing the clusters for a difference in the known treatment-response phenotype.

Clause 17. The method of Clause 16, wherein the clusters show a difference in the known treatment-response phenotype with a p value of less than 0.05.

Clause 18. The method of any one of Clauses 15-17, wherein generating at least one single-sample signature for each validation subject in the group comprises generating a signed single-sample signature and an absolute valued single-sample signature.

Clause 19. A treatment-response biomarker identification system, comprising:

one or more processors; and

memory coupled to the one or more processors, wherein the memory comprises computer-executable instructions causing the one or more processors to perform a process comprising:
(i) receiving genomic and epigenomic datasets for at least two subjects, wherein the at least two subjects have different treatment-response phenotypes;
(ii) identifying differential genomic and epigenomic datapoints in the datasets, wherein identifying generates at least one differential genomic signature for the treatment-response phenotypes and at least one differential epigenomic signature for the treatment-response phenotypes;
(iii) determining biological pathways enriched in the at least one differential genomic signature and the at least one differential epigenomic signature, wherein the determining generates at least one genomic pathway signature and at least one epigenomic pathway signature; and
(iv) selecting pathways enriched between the at least one genomic pathway signature and the at least one epigenomic pathway signature, wherein the selecting generates a comprehensive pathway signature for the treatment-response phenotypes.

Clause 20. One or more computer-readable media having encoded thereon computer-executable instructions that, when executed, cause a computing system to perform a treatment-response biomarker identification method comprising:

(i) receiving genomic and epigenomic datasets for at least two subjects, wherein the at least two subjects have different treatment-response phenotypes;

(ii) identifying differential genomic and epigenomic datapoints in the datasets, wherein the identifying generates at least one differential genomic signature for the treatment-response phenotypes and at least one differential epigenomic signature for the treatment-response phenotypes;

(iii) determining biological pathways enriched in the at least one differential genomic signature and the at least one differential epigenomic signature, wherein the determining generates at least one genomic pathway signature and at least one epigenomic pathway signature; and

(iv) selecting pathways enriched between the at least one genomic pathway signature and the at least one epigenomic pathway signature, wherein the selecting generates a comprehensive pathway signature for the treatment-response phenotypes.

Clause 21. The method of claim 1, wherein:

generating the at least one genomic pathway signature comprises, for a given input pathway, performing gene set enrichment analysis with genes in the given input pathway as a query and a treatment response signature as a reference.

Clause 22. The method of any one of claims 1-2, wherein:

selecting biological pathways enriched comprises performing, for a given analyzed pathway, pathway-on-pathway gene set enrichment analysis with the following as query and reference:

a composite methylation pathway signature for the given analyzed pathway; and

a composite expression pathway signature for the given analyzed pathway.

### Example 54—Example Alternatives

The technologies from any example can be combined with the technologies described in any one or more of the other examples. In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A computer-implemented method of identifying treatment-response biomarkers, the method comprising:
(i) receiving genomic and epigenomic datasets for at least two subjects, wherein the at least two subjects have different treatment-response phenotypes;
(ii) identifying differential genomic and epigenomic datapoints in the datasets, wherein the identifying generates at least one differential genomic signature for the treatment-response phenotypes and at least one differential epigenomic signature for the treatment-response phenotypes;
(iii) determining biological pathways enriched in the at least one differential genomic signature and the at least one differential epigenomic signature, wherein the determining generates at least one genomic pathway signature and at least one epigenomic pathway signature; and
(iv) selecting biological pathways enriched between the at least one genomic pathway signature and the at least one epigenomic pathway signature, wherein the selecting generates a comprehensive pathway signature for the treatment-response phenotypes.

2. The method of claim 1, wherein:
generating the at least one genomic pathway signature comprises, for a given input pathway, performing gene set enrichment analysis with genes in the given input pathway as a query and a treatment response signature as a reference.

3. The method of claim 1, wherein:
selecting biological pathways enriched comprises performing, for a given analyzed pathway, pathway-on-pathway gene set enrichment analysis with the following as query and reference:
a composite methylation pathway signature for the given analyzed pathway; and
a composite expression pathway signature for the given analyzed pathway.

4. The method of claim 1, wherein:
the at least one differential genomic signature for the treatment-response phenotypes comprises a signed differential genomic signature for the treatment-response phenotypes and/or an absolute valued differential genomic signature for the treatment-response phenotypes; and/or
the at least one differential epigenomic signature for the treatment-response phenotypes comprises a signed differential epigenomic signature for the treatment-response phenotypes and/or an absolute valued differential epigenomic signature for the treatment-response phenotypes.

5. The method of claim 4, wherein the at least one genomic pathway signature and at least one epigenomic pathway signature comprises:
a signed genomic pathway signature and/or an absolute valued genomic pathway signature; and/or
a signed epigenomic pathway signature and/or an absolute valued epigenomic pathway signature.

6. The method of claim 5, wherein the at least one genomic pathway signature and at least one epigenomic pathway signature comprises:
a signed genomic pathway signature and an absolute valued genomic pathway signature; and/or
a signed epigenomic pathway signature and an absolute valued epigenomic pathway signature.

7. The method of claim 6, further comprising:
combining the signed genomic pathway signature and the absolute valued genomic pathway signature, wherein the combining generates a composite genomic pathway signature; and/or
combining the signed epigenomic pathway signature and the absolute valued epigenomic pathway signature, wherein the combining generates a composite epigenomic pathway signature.

8. The method of claim 7, wherein combining comprises selecting for a highest pathway enrichment.

9. The method of claim 1, wherein:
the genomic dataset comprises gene expression datapoints; and
the epigenomic dataset comprises gene methylation datapoints.

10. The method of claim 1, wherein the at least 2 subjects comprise subjects that have cancer, and the different treatment-response phenotypes comprise a positive response to chemotherapy treatment and a negative response to chemotherapy treatment.

11. The method of claim 1, wherein the at least one differential genomic signature for the treatment-response phenotypes and/or the at least one differential epigenomic signature for the treatment-response phenotypes comprises genes ranked based on level of differentiation.

12. The method of claim 7, wherein the selecting biological pathways comprises removing pathways from the composite genomic pathway signature and the composite epigenomic pathway signature that are not associated with both differential genomic and epigenomic datapoints.

13. The method of claim 7, wherein the selecting biological pathways comprises removing pathways from the composite genomic pathway signature and the composite epigenomic pathway signature that are not enriched with a p value of less than 0.001.

14. The method of claim 1, further comprising validating clinical significance, non-randomness, and/or accuracy of the comprehensive pathway signature.

15. The method of claim 14, wherein validating the clinical significance comprises:
receiving genomic and epigenomic datasets for a group of validation subjects, wherein treatment-response phenotypes are known;
identifying differential genomic and epigenomic datapoints in the datasets, wherein the identifying generates at least one single-sample signature for each validation subject in the group;
determining enrichment of biological pathways from the comprehensive pathway signature in the at least one single-sample signature for each validation subject in the group, wherein the determining generates a pathway activity signature for each validation subject;
clustering the validation subjects in the group into treatment-response clusters based on the pathway activity signature for each validation subject; and
comparing the treatment response clusters with the known treatment-response phenotypes.

16. The method of claim 15, wherein comparing the treatment response clusters with the known treatment-response phenotypes comprises statistically analyzing the clusters for a difference in the known treatment-response phenotype.

17. The method of claim 16, wherein the clusters show a difference in the known treatment-response phenotype with a p value of less than 0.05.

18. The method of claim 15, wherein generating at least one single-sample signature for each validation subject in the group comprises generating a signed single-sample signature and an absolute valued single-sample signature.

19. A treatment-response biomarker identification system, comprising:
one or more processors; and
memory coupled to the one or more processors, wherein the memory comprises computer-executable instructions causing the one or more processors to perform a process comprising:
(i) receiving genomic and epigenomic datasets for at least two subjects, wherein the at least two subjects have different treatment-response phenotypes;
(ii) identifying differential genomic and epigenomic datapoints in the datasets, wherein identifying generates at least one differential genomic signature for the treatment-response phenotypes and at least one differential epigenomic signature for the treatment-response phenotypes;
(iii) determining biological pathways enriched in the at least one differential genomic signature and the at least one differential epigenomic signature, wherein the determining generates at least one genomic pathway signature and at least one epigenomic pathway signature; and
(iv) selecting pathways enriched between the at least one genomic pathway signature and the at least one epigenomic pathway signature, wherein the selecting generates a comprehensive pathway signature for the treatment-response phenotypes.

20. One or more computer-readable media having encoded thereon computer-executable instructions that, when executed, cause a computing system to perform a treatment-response biomarker identification method comprising:
(i) receiving genomic and epigenomic datasets for at least two subjects, wherein the at least two subjects have different treatment-response phenotypes;
(ii) identifying differential genomic and epigenomic datapoints in the datasets, wherein the identifying generates at least one differential genomic signature for the treatment-response phenotypes and at least one differential epigenomic signature for the treatment-response phenotypes;
(iii) determining biological pathways enriched in the at least one differential genomic signature and the at least one differential epigenomic signature, wherein the determining generates at least one genomic pathway signature and at least one epigenomic pathway signature; and
(iv) selecting pathways enriched between the at least one genomic pathway signature and the at least one epigenomic pathway signature, wherein the selecting generates a comprehensive pathway signature for the treatment-response phenotypes.
